(19) United States
(12) Patent Application Publication
(10) Pub. No.: US 2023/0398260 A1
(43) Pub. Date: Dec. 14, 2023

(54) METHODS FOR PROCESSING FETAL SUPPORT TISSUE

(71) Applicant: BioTissue Holdings Inc., Miami, FL (US)

(72) Inventors: Scheffer TSENG, Pinecrest, FL (US); Ek Kia TAN, Miami, FL (US); Lorraine CHUA, Miami, FL (US)

(21) Appl. No.: 18/033,470

(22) PCT Filed: Oct. 25, 2021
(86) PCT No.: PCT/US2021/056518

§ 371 Date: Apr. 24, 2023

Related U.S. Application Data
(60) Provisional application No. 63/105,770, filed on Oct. 26, 2020.

(51) Int. Cl. A61L 27/36; C12N 5/071; A61L 27/20; A61L 26/00

(52) U.S. Cl. CPC A61L 27/3691; C12N 5/0697; A61L 27/3604; A61L 27/20; A61L 26/0057; A61L 27/365; A61L 2430/38; A61L 2430/24

(57) ABSTRACT

Disclosed herein, in certain embodiments are methods of preparing a fetal support tissue product comprising: cryopulverizing or homogenizing a fetal support tissue, extracting the cryopulverized fetal support tissue in an excipient, and sterilizing the extract. Also disclosed herein are pharmaceutical compositions comprising said fetal support tissue product and methods of using said fetal support tissue product for treating wounds, spinal conditions, and arthritis.

### CROSS REFERENCE

This application claims the benefit of U.S. Application No. 63/105,770, filed Oct. 26, 2020, which is hereby incorporated by reference in its entirety.

### SUMMARY

Disclosed herein are methods of preparing a fetal support tissue product, comprising: (a) cryopulverizing the fetal support tissue to generate a cryopulverized fetal support tissue; (b) extracting the cryopulverized fetal support tissue in an excipient to generate an extract; and (c) sterilizing by filtration the extract using a membrane having a pore size of about 0.6 μm or less followed by using a membrane having a pore size of about 0.4 μm or less; wherein the fetal support tissue product is produced. In some embodiments, the sterilizing by filtration is using a membrane having a pore size of about 0.45 μm followed by using a membrane having a pore size of about 0.2 μm or less. In some embodiments, the cryopulverizing comprises pulverizing the fetal support tissue in liquid nitrogen. In some embodiments, the cryopulverizing comprises pulverizing the fetal support tissue to a fine powder. In some embodiments, the fetal support tissue comprises placenta, umbilical cord, placental amniotic membrane umbilical cord amniotic membrane, chorion, or amnion-chorion, or any combinations thereof. In some embodiments, the fetal support tissue comprises umbilical cord and placental amniotic membrane. In some embodiments, the excipient is saline, water for injection (WFI), or any combination thereof. In some embodiments, the excipient is WFI. In some embodiments, the excipient is saline. In some embodiments, the methods further comprise following step c) centrifuging the fetal support tissue. In some embodiments, the centrifuge speed is about 14,000 relative centrifugal force (rcf) or higher. In some embodiments, the methods further comprise diluting the fetal support tissue with an excipient following centrifugation. In some embodiments, the excipient is WFI or saline. In some embodiments, the excipient is WFI. In some embodiments, the excipient is saline. In some embodiments, the fetal support tissue is diluted by a factor of at least about 1.5-, 2.0-, or 2.5-fold. In some embodiments, the fetal support tissue is diluted by a factor of between about 1.5- to about 3-fold, by a factor of between about 3-fold to about 5-fold, or by a factor of between about 5-fold to 10-fold. In some embodiments, the fetal support tissue is diluted by a factor greater than 5-fold. In some embodiments, the fetal support tissue is diluted by a factor greater than 10-fold. In some embodiments, the fetal support tissue is diluted by a factor of about 2-fold. In some embodiments, the diluted fetal support tissue comprises from about 1 μg/ml to about 150 μg/ml of Hyaluronan (HA). In some embodiments, the fetal support tissue product is anti-inflammatory, anti-scarring, anti-angiogenic, anti-adhesion, or promotes wound healing. In some embodiments, the method comprises pooling the fetal support tissue product with at least one additional fetal support tissue product. In some embodiments, the fetal support tissue product and the at least one additional fetal support tissue product comprise fetal support tissues derived from at least two different subjects. In some embodiments, the fetal support tissue product and the at least one additional fetal support tissue product comprise fetal support tissues derived from at least five different subjects. In some embodiments, the fetal support tissue product and the at least one additional fetal support tissue product comprise fetal support tissues derived from at least fifteen different subjects. In some embodiments, the fetal support tissue product and the at least one additional fetal support tissue product comprise fetal support tissues derived from at least forty-five different subjects. In some embodiments, the method comprises filling the fetal support tissue product into a container. In some embodiments, the method comprises sealing the container. In some embodiments, the filling and sealing are carried out aseptically. In some embodiments, the filling and sealing are carried out aseptically and in a single continuous process without human intervention.

Disclosed herein, in certain embodiments, is a method of preparing a fetal support tissue product, comprising: (a) homogenizing the fetal support tissue to generate a homogenized fetal support tissue; (b) extracting the homogenized fetal support tissue in an excipient to generate an extract; and (c) sterilizing the extract using gamma irradiation or electron beam sterilization, wherein the fetal support tissue product is produced. In some embodiments, the homogenizing comprises pulverizing the fetal support tissue to a fine powder. In some embodiments, the fetal support tissue comprises placenta, umbilical cord, placental amniotic membrane umbilical cord amniotic membrane, chorion, or amnion-chorion, or any combinations thereof. In some embodiments, the fetal support tissue comprises umbilical cord and placental amniotic membrane. In some embodiments, the excipient is saline, water for injection (WFI), or any combination thereof. In some embodiments, the excipient is WFI. In some embodiments, the excipient is saline. In some embodiments, the method further comprises, following step c) centrifuging the fetal support tissue. In some embodiments, the centrifuge speed is about 14,000 relative centrifugal force (rcf) or higher. In some embodiments, the method further comprises diluting the fetal support tissue with an excipient following centrifugation. In some embodiments, the excipient is WFI or saline. In some embodiments, the excipient is WFI. In some embodiments, the excipient is saline. In some embodiments, the fetal support tissue is diluted by a factor of at least about 1.5-, 2.0-, or 2.5-fold. In some embodiments, the fetal support tissue is diluted by a factor of between about 1.5- to about 3-fold, by a factor of between about 3-fold to about 5-fold, or by a factor of between about 5-fold to 10-fold. In some embodiments, the fetal support tissue is diluted by a factor greater than 5-fold. In some embodiments, the fetal support tissue is diluted by a factor greater than 10-fold. In some embodiments, the fetal support tissue is diluted by a factor of about 2-fold. In some embodiments, the diluted fetal support tissue comprises from about 1 μg/ml to about 150 μg/ml of Hyaluronan (HA). In some embodiments, the fetal support tissue product is anti-inflammatory, anti-scarring, anti-angiogenic, anti-adhesion, or promotes wound healing. In some embodiments, the method comprises pooling the fetal support tissue product with at least one additional fetal support tissue product. In some embodiments, the fetal support tissue product and the at least one additional fetal support tissue product comprise fetal support tissues derived from at least two different subjects. In some embodiments, the fetal support tissue product and the at least one additional fetal support tissue product comprise fetal support tissues derived from at least five different subjects. In some embodiments, the fetal support tissue product and the at least one additional fetal support tissue product comprise fetal support tissues derived from at least fifteen different subjects. In some embodiments, the fetal support tissue product and the at least one additional fetal support tissue product comprise fetal support tissues derived from at least forty-five different subjects. In some embodiments, the method comprises filling the fetal support tissue product into a container. In some embodiments, the method comprises sealing the container. In some embodiments, the filling and sealing are carried out aseptically. In some embodiments, the filling and sealing are carried out aseptically and in a single continuous process without human intervention.

Disclosed herein, in certain embodiments, is a pharmaceutical composition, comprising (a) the fetal support tissue product made by any of the methods disclosed herein, and (b) a pharmaceutically-acceptable carrier. In some embodiments, the pharmaceutically-acceptable carrier is selected from: carbomer, cellulose, collagen, glycerin, hexylene glycol, hyaluronic acid, hydroxypropyl cellulose, phosphoric acid, polysorbate 80, propylene glycol, propylene glycol stearate, saline, sodium hydroxide, sodium phosphate, sorbital, water, xanthan gum, or any combination thereof. In some embodiments, the fetal support tissue powder product is administered or provided as a cream, lotion, ointment, ophthalmic solution, spray, paste, gel, film, or paint. In some embodiments, the pharmaceutical composition is anti-inflammatory, anti-scarring, anti-angiogenic, anti-adhesion, or promotes wound healing.

Disclosed herein, in certain embodiments, is a method of treating a wound in an individual in need thereof, comprising administering any of the pharmaceutical compositions disclosed herein to the wound for a period of time sufficient to treat the wound. In some embodiments, the wound is a corneal epithelial wound. In some embodiments, the corneal epithelial wound was caused by a photoablation treatment. In some embodiments, the wound is a dermatological condition selected from a dermal burn or a scar.

Disclosed herein, in certain embodiments, is a method of treating a spinal condition in an individual in need thereof, comprising administering any of the pharmaceutical compositions disclosed herein to the individual for a period of time sufficient to treat the spinal condition. In some embodiments, the spinal condition is selected from a herniated disc, spinal adhesion, facet joint osteoarthritis, radiculopathy, or discitis. In some embodiments the spinal condition is a spinal cord injury.

Disclosed herein, in certain embodiments, is a method of treating an arthritic condition in an individual in need thereof, comprising administering any of the pharmaceutical compositions disclosed herein to the individual for a period of time sufficient to treat the arthritic condition. In some embodiments, the arthritic condition is selected from osteoarthritis, rheumatoid arthritis, septic arthritis, ankylosing spondylitis, or spondylosis.

Disclosed herein, in certain embodiments, is a method of regenerating or repairing bone, tissue or cartilage in an individual in need thereof, comprising administering or providing any of the pharmaceutical compositions disclosed herein to the individual for a period of time sufficient to regenerate or repair bone, tissue or cartilage. In some embodiments, the pharmaceutical composition is administered or provided as a patch. In some embodiments, the pharmaceutical composition is administered or provided as a wound dressing.

### BRIEF DESCRIPTION OF THE FIGURES

FIG. 1 shows a flow chart illustrating an example of a method of producing a fetal support tissue product disclosed herein.

FIGS. 2A-2D show a Western Blot analysis of sequential saline and GnHCl extracts of a cryopulverized fetal support tissue after 1 hour of extraction.

FIGS. 3A-3B show a Western blot analysis of a fetal support tissue extracted in saline and centrifuged at different centrifuge speeds.

FIG. 4 shows an agarose gel analysis of sequential saline, water for injection (“WFI”) or sterile water (“SW”) and GnHCl extracts of a cryopulverized fetal support tissue.

FIGS. 5A-5B show a Western blot analysis of sequential saline, WFI or SW and GnHCl extracts of a cryopulverized fetal support tissue.

FIG. 6 shows an agarose gel analysis of a fetal support tissue product after gamma-irradiation treatment.

FIGS. 7A-7B show a Coomassie blue analysis of a fetal support tissue product after gamma-irradiation treatment.

FIGS. 8A-8D show a Western blot analysis of a fetal support tissue extract after gamma-irradiation.

FIG. 9 shows an agarose gel analysis of a fetal support tissue extract after filtration sterilization and dilution.

FIGS. 10A-10D show a Western blot analysis of a fetal support tissue extract after filtration sterilization and dilution.

FIGS. 11A-11C show dose-dependent linearity analysis of Hyaluronan (“HA”). concentration in three potency assays—(1) a TRAP assay; (2) a M2 assay; and (3) a WST-1 assay.

FIG. 12 shows a flow chart illustrating the storage, transportation, and terminal sterilization of a fetal support tissue product.

FIGS. 13A-13F show an agarose gel analysis of fetal support tissue products produced in varied excipients with and without gamma irradiation.

FIGS. 14A-14B show a Coomassie blue stain analysis of fetal support tissue products produced in varied excipients with and without gamma irradiation.

FIGS. 15A-15J show a western blot analysis of fetal support tissue products produced in varied excipients with and without gamma irradiation.

FIGS. 16A-16B show cells morphology images of fetal support tissue products produced in varied excipients with and without gamma irradiation.

FIGS. 17A-17E show a TRAP analysis of fetal support tissue products produced in varied excipients with and without gamma irradiation.

FIGS. 18A-18B show an agarose gel analysis of fetal support tissue products.

FIGS. 19A-19D show Western blot analysis of fetal support tissue products.

FIGS. 20A-20B show a cell morphology analysis and ODITRAP assay of fetal support tissue products.

FIGS. 21A-21B show a cell morphology analysis and M2 assay of fetal support tissue products.

FIGS. 22A-22B show a cell morphology analysis and NO assay of fetal support tissue products.

FIG. 23 shows a flow chart illustrating an example of a method of producing a fetal support tissue product disclosed herein.

FIG. 24A-24C a flow chart illustrating an example of a method of producing a fetal support tissue product by pooling multiple donors to increase yield disclosed herein.

### DETAILED DESCRIPTION

Amniotic membrane and umbilical cord contain several innate biological factors useful for a number of purposes, including wound healing and reducing inflammation and scarring. The HC-HA/PTX3 complex—high molecular weight (HMW) hyaluronan (HA) covalently linked with heavy chain (HC) 1 from inter-α-trypsin inhibitor and further complexed with pentraxin3 (PTX3)—is one key active component of umbilical cord and amniotic membrane that is responsible for their wound healing effects. Accordingly, production of a fetal support tissue product (e.g., an amniotic membrane and umbilical cord extract for use in wound healing) with a high yield of HC-HA/PTX3, HA, and other proteins of interest is needed. Production of a fetal support tissue product using a process that reduces or prevents the degradation of the HC-HA/PTX3 complex and other proteins of interest is also needed. Preventing degradation of HA and other proteins of interest is important because the degradation of such proteins can render a fetal support tissue product unsuitable for use, for example, on the corneal surface where the smaller particulates produced by degradation may result in blurred vision.

Disclosed herein, in certain embodiments, are methods of preparing a fetal support tissue product comprising: (a) cryopulverizing a fetal support tissue to generate a cryopulverized fetal support tissue; (b) extracting the cryopulverized fetal support tissue in an excipient to generate an extract; and (c) sterilizing by filtration the extract using a membrane having a pore size of about 0.6 μm or less followed by using a membrane having a pore size of about 0.3 μm or less. In some embodiments, the fetal support tissue is placental amniotic membrane (PAM), or substantially isolated PAM, umbilical cord amniotic membrane (UCAM) or substantially isolated UCAM, chorion or substantially isolated chorion, amnion-chorion or substantially isolated amnion-chorion, placenta or substantially isolated placenta, umbilical cord or substantially isolated umbilical cord, or any combinations thereof. In some embodiments, cryopulverizing comprises pulverizing the fetal support tissue in liquid nitrogen. In some embodiments, cryopulverizing comprises pulverizing the fetal support tissue to a fine powder. In some embodiments, the excipient is saline, water for injection (WFI), or any combination thereof. In some embodiments, the method further comprises centrifuging the fetal support tissue. In some embodiments, the centrifuge speed is about 14,000 relative centrifugal force (rcf) or higher. In some embodiments, the method further comprises diluting the fetal support tissue with an excipient following centrifugation. In some embodiments, the excipient is WFI or saline. In some embodiments, the fetal support tissue is diluted by a factor of at least about 1.5-, 2.0-, or 2.5-fold In some embodiments, the fetal support tissue is diluted by a factor of between about 1.5- to about 3-fold, by a factor of between about 3-fold to about 5-fold, or by a factor of between about 5-fold to 10-fold. In some embodiments, the fetal support tissue is diluted by a factor greater than 5-fold. In some embodiments, the fetal support tissue is diluted by a factor greater than 10-fold. In some embodiments, diluted fetal support tissue comprises from about 1 μg/mL—to about 150 μg/mL of Hyaluronan (HA). In some embodiments, diluted fetal support tissue comprises from about 1 μg/mL— to about 90 μg/mL of HA. In some embodiments, diluted fetal support tissue comprises from about 90 μg/ml—to about 150 μg/ml of HA. In some embodiments, diluted fetal support tissue comprises from about 1-10 μg/mL of HA, about 10-20 μg/mL of HA, about 20-30 μg/mL of HA, about 30-40 μg/mL of HA, about 40-50 μg/mL of HA, about 50-60 μg/mL of HA, about 60-70 μg/mL of HA, about 70-80 μg/mL of HA, about 80-90 μg/mL of HA, about 90-100 μg/mL of HA, about 100-110 μg/mL of HA, about 110-120 μg/mL of HA, about 120-130 μg/mL of HA, about 130-140 μg/mL of HA, or about 140-150 μg/mL of HA.

In some instances, cryopulverization of the fetal support tissue produces a fetal support tissue product with a higher yield of HC-HA/PTX3 and other proteins, as compared to methods of production which use other homogenization techniques. In some cases, cryopulverization of the fetal support tissue results in less degradation of HC-HA/PTX3 and other proteins of interest, as compared to methods of production using other homogenization techniques. In some cases, cryopulverization of the fetal support tissue produces a fetal support tissue product of higher potency, as compared to methods of production using other homogenization techniques, for example as determined by an ODI-TRAP assay, an M2 assay, a Nitric Oxide (NO) assay, and/or a WST-1 assay. In some cases, sterilization by filtration results in less degradation of HC-HA/PTX3, HA and other proteins of interest, as compared to a method of production where the fetal support tissue product is sterilized by gamma irradiation. In some cases, sterilization by filtration removes smaller particles from a fetal support tissue product that may be undesirable in certain formulations (e.g., a gel comprising a fetal support tissue for use on the corneal surface). In some cases, diluting the fetal support tissue results in faster filtration and better recovery of HC-HA/PTX3 and other proteins of interest, as compared to a process that does not include a dilution step. In some cases, using water for injection as the excipient results in faster filtration and better recovery of HC-HA/PTX3 and other proteins of interest, as compared to a process that uses a different excipient, such as structured water.

Disclosed herein, in certain embodiments, are fetal support tissue products prepared by the method comprising: (a) cryopulverizing a fetal support tissue to generate a cryopulverized fetal support tissue; (b) extracting the cryopulverized fetal support tissue in an excipient to generate an extract; and (c) sterilizing by filtration the extract using a membrane having a pore size of about 0.6 μm followed by using a membrane having a pore size of about 0.4 μm or less. In some embodiments, the fetal support tissue is placental amniotic membrane (PAM), or substantially isolated PAM, umbilical cord amniotic membrane (UCAM) or substantially isolated UCAM, chorion or substantially isolated chorion, amnion-chorion or substantially isolated amnion-chorion, placenta or substantially isolated placenta, umbilical cord or substantially isolated umbilical cord, or any combinations thereof.

Disclosed herein, in certain embodiments, is a pharmaceutical composition, comprising a fetal support tissue product disclosed herein and a pharmaceutically-acceptable carrier. In some embodiments, the pharmaceutically-acceptable carrier is selected from carbomer, cellulose, collagen, glycerin, hexylene glycol, hyaluronic acid, hydroxypropyl cellulose, phosphoric acid, polysorbate 80, propylene glycol, propylene glycol stearate, saline, sodium hydroxide, sodium phosphate, sorbital, water, xanthan gum, or any combination thereof. In some embodiments, the pharmaceutical composition is administered or provided as a cream, lotion, ointment, ophthalmic solution, spray, paste, gel, film, or paint. In some embodiments, the pharmaceutical composition is anti-inflammatory, anti-scarring, anti-angiogenic, anti-adhesion, or promotes wound healing. In some embodiments, the pharmaceutical composition is formulated for epidural administration, intrathecal administration, inhalational administration, intravenous administration, or a combination thereof.

Disclosed herein, in certain embodiments, are methods of treating a wound in an individual in need thereof, comprising administering a pharmaceutical composition disclosed herein to the wound for a period of time sufficient to treat the wound. In some embodiments, the wound is a dermatological condition selected from a dermal burn or a scar. In some embodiments, the pharmaceutical composition is administered or provided as a patch. In some embodiments, the pharmaceutical composition is administered or provided as a wound dressing. In some embodiments, the pharmaceutical composition is formulated for injection. In some embodiments, the pharmaceutical composition is formulated for parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the pharmaceutical composition is formulated for epidural administration, intrathecal administration, inhalational administration, intravenous administration, or a combination thereof.

Disclosed herein, in certain embodiments, are methods of treating a spinal condition in an individual in need thereof, comprising administering a pharmaceutical composition disclosed herein to the individual for a period of time sufficient to treat the spinal condition. In some embodiments, the spinal condition is selected from a herniated disc, spinal adhesion, facet joint osteoarthritis, radiculopathy, or discitis. In some embodiments the spinal condition is a spinal cord injury. In some embodiments, the pharmaceutical composition is administered or provided as a patch. In some embodiments, the pharmaceutical composition is administered or provided as a wound dressing. In some embodiments, the pharmaceutical composition is formulated for injection. In some embodiments, the pharmaceutical composition is formulated for parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the pharmaceutical composition is formulated for epidural administration, intrathecal administration, inhalational administration, intravenous administration, or a combination thereof.

Disclosed herein, in certain embodiments, are methods of treating an arthritic condition in an individual in need thereof, comprising administering a pharmaceutical composition disclosed herein to the individual for a period of time sufficient to treat the arthritic condition. In some embodiments, the arthritic condition is selected from osteoarthritis, rheumatoid arthritis, septic arthritis, ankylosing spondylitis, or spondylosis. In some embodiments, the pharmaceutical composition is administered or provided as a patch. In some embodiments, the pharmaceutical composition is administered or provided as a wound dressing. In some embodiments, the pharmaceutical composition is formulated for injection. In some embodiments, the pharmaceutical composition is formulated for parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the pharmaceutical composition is formulated for epidural administration, intrathecal administration, inhalational administration, intravenous administration, or a combination thereof.

Disclosed herein, in certain embodiments, are methods of regenerating or repairing bone, tissue or cartilage in an individual in need thereof, comprising administering or providing a pharmaceutical composition disclosed herein to the individual for a period of time sufficient to regenerate or repair bone, tissue or cartilage. In some embodiments, the pharmaceutical composition is administered or provided as a patch. In some embodiments, the pharmaceutical composition is administered or provided as a wound dressing. In some embodiments, the pharmaceutical composition is formulated for injection. In some embodiments, the pharmaceutical composition is formulated for parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the pharmaceutical composition is formulated for epidural administration, intrathecal administration, inhalational administration, intravenous administration, or a combination thereof.

### Preparation of Fetal Support Tissue Product

### Initial Processing

Fetal support tissue is obtained from any suitable source (e.g., a hospital or tissue bank). In some embodiments, fetal support tissue is obtained from any mammal, such as a human, non-human primate, cow or pig.

In some embodiments, the fetal support tissue is frozen (e.g., at or below 0° C.) until donor and specimen eligibility has been determined. In some embodiments, freezing the fetal support tissue kills substantially all cells found in the fetal support tissue. In some embodiments, freezing the fetal support tissue kills substantially all cells found in fetal support tissue while maintaining or increasing the biological activity of the fetal support tissue relative to fresh (i.e., non-frozen) fetal support tissue. In some embodiments, freezing the fetal support tissue results in the loss of metabolic activity in substantially all cells found in the fetal support tissue. In some embodiments, freezing the fetal support tissue results in the loss of metabolic activity in substantially all cells found in the fetal support tissue while maintaining or increasing the biological activity of the fetal support tissue (e.g., its anti-inflammatory, anti-scarring, anti-antigenic, and anti-adhesion properties) relative to fresh (i.e., non-frozen) fetal support tissue.

In some embodiments, the fetal support tissue is not frozen. If the fetal support tissue is not frozen, it is processed as described below immediately.

In some embodiments, the processing is done following Good Tissue Practices (GTP) to ensure that no contaminants are introduced into the fetal support tissue powder products.

In some embodiments, the fetal support tissue is tested for HIV-1, HIV-2, HTLV-1, hepatitis B and C, West Nile virus, cytomegalovirus, human transmissible spongiform encephalopathy (e.g., Creutzfeldt-Jakob disease) and Treponema pallidum using FDA licensed screening test. In some embodiments, any indication that the tissue is contaminated with HIV-1, HIV-2, HTLV-1, hepatitis B and C, West Nile virus, or cytomegalovirus results in the immediate quarantine and subsequent destruction of the tissue specimen.

In some embodiments, the donor's medical records are examined for risk factors for and clinical evidence of hepatitis B, hepatitis C, or HIV infection. In some embodiments, any indication that the donor has risk factors for, and/or clinical evidence of, infection with HIV-1, HIV-2, HTLV-1, hepatitis B and C, West Nile virus, cytomegalovirus, human transmissible spongiform encephalopathy (e.g., Creutzfeldt-Jakob disease) and Treponema pallidum results in the immediate quarantine and subsequent destruction of the tissue specimen.

In some embodiments, substantially all of blood is removed from the fetal support tissue. In some embodiments, substantially all blood is removed from the fetal support tissue before the fetal support tissue is frozen.

In some embodiments, blood is not removed from the fetal support tissue. In some embodiments, blood is not removed from the fetal support tissue before the fetal support tissue is frozen.

In some embodiments, the fetal support tissue is contacted with an isotonic buffer. In some embodiments, the fetal support tissue is contacted with saline, PBS, PBS 1×, Ringer's solution, Hartmann's solution, TRIS-buffered saline, HEPES-buffered saline, EBSS, HBSS, Tyrode's salt Solution, Gey's Balanced Salt Solution, DMEM, EMEM, GMEM, RPMI, or any combinations thereof.

In some embodiments, the fetal support tissue is washed with buffer with agitation to remove excess blood and tissue. In some embodiments, washing with agitation reduces the wash time.

In some embodiments, the fetal support tissue is placental amniotic membrane (PAM), or substantially isolated PAM, umbilical cord amniotic membrane (UCAM) or substantially isolated UCAM, chorion or substantially isolated chorion, amnion-chorion or substantially isolated amnion-chorion, placenta or substantially isolated placenta, umbilical cord or substantially isolated umbilical cord, or any combinations thereof.

In some embodiments, the fetal support tissue is umbilical cord or umbilical cord amniotic membrane. In some embodiments, the Wharton's Jelly is not removed from the umbilical cord or the umbilical cord amniotic membrane. In some embodiments, part or all of the Wharton's Jelly is removed from the umbilical cord or the umbilical cord amniotic membrane.

Umbilical cord comprises two arteries (the umbilical arteries) and one vein (the umbilical vein). In certain instances, the vein and arteries are surrounded (or suspended or buried) within the Wharton's Jelly. In some embodiments, the veins and arteries are not removed from the umbilical cord. In some embodiments, the vein and arteries are removed from the umbilical cord. In some embodiments, the vein and arteries are removed concurrently with the removal of the Wharton's Jelly.

### Grinding/Cryopulverization

In some embodiments, the fetal support tissue is ground by any suitable method. In some embodiments, grinding the fetal support tissue comprises cryopulverizing the fetal support tissue. In some embodiments, cryopulverizing fetal support tissue comprises pulverizing, homogenizing, or otherwise fragmenting the fetal support tissue while the fetal support tissue is in a frozen (e.g., exposed to a temperature below 0° C., −20° C., −40° C., −50° C., −60° C., −70° C., −75° C., −80° C., −90° C., −100° C.) or chilled state. In some embodiments, cryopulverizing the fetal support tissue comprises pulverizing or homogenizing the fetal support tissue in a cryogenically controlled environment. In some embodiments, cryopulverizing the fetal support tissue comprises pulverizing or homogenizing the fetal support tissue after the fetal support tissue has been immersed in or exposed to (e.g., directly or indirectly) liquid nitrogen. In some embodiments, cryopulverizing the fetal support tissue comprises pulverizing or homogenizing the fetal support tissue while the fetal support tissue is immersed in or exposed to (e.g., directly or indirectly) liquid nitrogen. In some embodiments, cryopulverizing the fetal support tissue comprises placing the fetal support tissue in a grinding container and immersing the grinding container in liquid nitrogen prior to grinding. In some embodiments, the grinding container is immersed in liquid nitrogen for at least 1 minute of the grinding process. In some embodiments, cryopulverizing fetal support tissue comprises exposing frozen fetal support tissue to a hammer or rotating blade. In some embodiments, cryopulverizing fetal support tissue comprises exposing frozen fetal support tissue to an impactor. In some embodiments, the impactor is driven by electromagnets. In some embodiments, the fetal support tissue is cryopulverized by use of a FreezerMill. In some embodiments, the fetal support tissue is cryopulverized by use of a mortar and pestle. In some embodiments, the fetal support tissue is cryopulverized by use of a blender. In some embodiments, the fetal support tissue is cryopulverized by use of a BioPulverizer. In some embodiments, cryopulverizing fetal support tissue in liquid nitrogen, as compared to grinding fetal support tissue that has not been frozen, avoids activating in the fetal support tissue protease and/or hyaluronidase, which may degrade proteins and hyaluronan in the fetal support tissue product.

In some embodiments, cryopulverization reduces the fetal support tissue to a powder. In some embodiments, the particles comprising the powder are of uniform size distribution. In some embodiments, the particles comprising the powder are not of uniform size distribution. In some embodiments, the fetal support tissue is reduced to a particle size of less than about 1000 μm, 500 μm, 400 μm, 300 μm, 200 μm, 100 μm, 50 μm, 40 μm, 30 μm, 20 μm, 10 μm, 5 μm, 1 μm, 0.5 μm, 0.4 μm, 0.3 μm, 0.2 μm, or 0.1 μm. In some embodiments, the fetal support tissue is reduced to a particle size of less than 500 μm. In some embodiments, the fetal support tissue is reduced to a particle size of less than about 0.5 μm. In some embodiments, the fetal support tissue is reduced to a particle size of less than 0.3 μm.

### Extraction and Centrifugation

In some embodiments, extraction is performed on the fetal support tissue. In some embodiments, extraction comprises separating proteins of interest from other components of the fetal support tissue. In some embodiments, extraction is performed on the cryopulverized fetal support tissue. In some embodiments, extraction comprises separating proteins of interest from other components of the cryopulverized fetal support tissue. In some embodiments, such proteins of interest comprise proteins, glycans, protein-glycan complexes (e.g., a complex of hyaluronan and a heavy chain of IαI and PTX3) and enzymes that promote tissue repair. In some embodiments, such proteins of interest comprise a complex of hyaluronan and a heavy chain of IαI and PTX3 (“HC-HA/PTX”), hyaluronan, high molecular weight hyaluronan, or a combination thereof. In some embodiments, such proteins of interest comprise hyaluronan.

In some embodiments, the extract is prepared by extraction in an excipient. In some embodiments, the excipient is saline, water, structured water, water for injection (WFI), or a combination thereof. In some embodiments, the excipient is WFI. In some embodiments, use of WFI as an excipient produces a higher recovery rate of HA and total proteins as compared to saline, or structured water. In some embodiments, extraction in saline or WFI extracts a majority of a protein of interest (e.g., HC-HA/PTX3).

In some embodiments, the extraction is performed for about 0 to 1 hours, about 1 to 2 hours, about 2 to 3 hours, about 3 to 4 hours, about 4 to 5 hours, about 5 to 6 hours, about 6 to 12 hours, about 12 hours to 24 hours, about 24 hours to 48 hours or about 48 hours to 72 hours. In some embodiments, the extraction is performed for about 1 hour. In some embodiments, the extraction is performed in WFI for about 0 to 1 hours, about 1 to 2 hours, about 2 to 3 hours, about 3 to 4 hours, about 4 to 5 hours, about 5 to 6 hours, about 6 to 12 hours, about 12 hours to 24 hours, about 24 hours to 48 hours or about 48 hours to 72 hours. In some embodiments, the extraction is performed in WFI for about 1 hour. In some embodiments, the extraction is performed in saline for about 0 to 1 hours, about 1 to 2 hours, about 2 to 3 hours, about 3 to 4 hours, about 4 to 5 hours, about 5 to 6 hours, about 6 to 12 hours, about 12 hours to 24 hours, about 24 hours to 48 hours or about 48 hours to 72 hours. In some embodiments, the extraction is performed in saline for about 1 hour.

In some embodiments the extraction is performed at a temperature of about 4° C. In some embodiments, the extraction is performed at a temperature of about 3° C., 4° C., 5° C., or 6° C. In some embodiments, the extraction is performed in WFI at a temperature of about 3° C., 4° C., 5° C., or 6° C. In some embodiments the extraction is performed in WFI at a temperature of about 4° C. In some embodiments, the extraction is performed in WFI for about 1 hour at a temperature of about 4° C. In some embodiments, the extraction is performed in WFI for about for about 0 to 1 hours, about 1 to 2 hours, about 2 to 3 hours, about 3 to 4 hours, about 4 to 5 hours, about 5 to 6 hours, about 6 to 12 hours, about 12 hours to 24 hours, about 24 hours to 48 hours or about 48 hours to 72 hours, at a temperature of about 4° C. In some embodiments, the extraction is performed in saline at a temperature of about 3° C., 4° C., 5° C., or 6° C. In some embodiments the extraction is performed in saline at a temperature of about 4° C. In some embodiments, the extraction is performed in saline for about for about 0 to 1 hours, about 1 to 2 hours, about 2 to 3 hours, about 3 to 4 hours, about 4 to 5 hours, about 5 to 6 hours, about 6 to 12 hours, about 12 hours to 24 hours, about 24 hours to 48 hours or about 48 hours to 72 hours, at a temperature of about 4° C. In some embodiments, the extraction is performed in saline for about 1 hour at a temperature of about 4° C.

In some embodiments, the ratio of fetal support tissue to extraction excipient is about 1:6, 1:5, 1:4, 1:3, 1:2, 1:1, 1:0.75, 1:0.5, or 1:0.25 (weight:volume). In some embodiments, the ratio of fetal support tissue to extraction excipient is about 6:1, 5:1, 4:1, 3:1, 2:1, 1:1, 0.75:1, 0.5:1, or 0.25:1 (weight:volume). In some embodiments, the ratio of fetal support tissue to extraction excipient is about 1:4. In some embodiments, the extraction is performed in WFI or saline. In some embodiment, the extraction is performed in WFI. In some embodiments, the extraction is performed for about 0 to 1 hours, about 1 to 2 hours, about 2 to 3 hours, about 3 to 4 hours, about 4 to 5 hours, about 5 to 6 hours, about 6 to 12 hours, about 12 hours to 24 hours, about 24 hours to 48 hours or about 48 hours to 72 hours. In some embodiments, the extraction is performed for about 1 hour. In some embodiments, the extraction is performed for at a temperature of about 3° C., 4° C., 5° C., or 6° C. In some embodiments, the extraction is performed for at a temperature of about 4° C. In some embodiments, the extraction is performed in WFI for about 1 hour at a temperature of about 4° C. at a ratio of fetal support tissue to excipient of about 1:4. In some embodiments, the extraction is performed in saline for about 1 hour at a temperature of about 4° C. at a ratio of fetal support tissue to excipient of about 1:4.

In some embodiments, the extraction is performed using a tube rotator. In some embodiments, the tube rotator rotates at a range of speed from about 5-10 rpm, 10-20 rpm, 20-30 rpm, 30-40 rpm, or 40-50 rpm. In some embodiments, the tube rotator rotates at a speed of about rpm. In some embodiments, the tube rotator rotates for about 0 to 1 hours, about 1 to 2 hours, about 2 to 3 hours, about 3 to 4 hours, about 4 to 5 hours, about 5 to 6 hours, about 6 to 12 hours, about 12 hours to 24 hours, about 24 hours to 48 hours or about 48 hours to 72 hours. In some embodiments, the tube rotator rotates for about 1 hour. In some embodiments, the tube rotator rotates at a temperature of about 3° C., 4° C., 5° C., or 6° C. In some embodiments, the tube rotator rotates at a temperature of about 4° C. In some embodiments, the excipient used is saline or WFI. In some embodiments, the excipient used is WFI. In some embodiments, the ratio of fetal support tissue to extraction excipient is about 1:6, 1:5, 1:4, 1:3, 1:2, 1:1, 1:0.75, 1:0.5, or 1:0.25 (weight:volume). In some embodiments, the ratio of fetal support tissue to extraction excipient is about 1:4. In some embodiments, the ratio of fetal support tissue to extraction excipient is about 6:1, 5:1, 4:1, 3:1, 2:1, 1:1, 0.75:1, 0.5:1, or 0.25:1 (weight:volume). In some embodiments, the extraction is performed in WFI for about 1 hour at a temperature of about 4° C. using a tube rotator at a speed of about 20 rpm. In some embodiments, the extraction is performed in saline for about 1 hour at a temperature of about 4° C. using a tube rotator at a speed of about 20 rpm.

In some embodiments, centrifugation is performed on the extract produced by the extraction step. In some embodiments, extract is centrifuged for about 5 to 10 minutes, about 10 to 15 minutes, about 15 to 20 minutes, about 20 to 30 minutes, about 30 minutes to 1 hour, or about 1 to 2 hours. In some embodiments, the extract is centrifuged for about 30 minutes. In some embodiments, the extract is centrifuged at a speed of about 3,200 rcf to 10,000 rcf, about to 14,000 rcf, about 14,000 to 32,000 rcf, or about 32,000 to about 48,000 rcf. In some embodiments, the extract is centrifuged at a speed of about 14,000 rcf or higher. In some embodiments, the extract is centrifuged at a speed of about 14,000 rcf. In some embodiments, the extract is centrifuged at a speed of about 14,000 rcf for about 30 minutes. In some embodiments, the centrifugation does not affect or minimally effects the content of the protein of interest in the extract.

In some embodiments the extraction produces an extract comprising the proteins of interest (e.g., hyaluronan or a complex of hyaluronan and a heavy chain of IαI and PTX3). In some embodiments, the majority of HC-HA/PTX3 is present in the extract. In some embodiments, the majority of HA is present in the extract. In some embodiments, the extraction in saline or WFI results in less damage to the HC-HA/PTX3 complex than extraction using a different excipient, for example structured water. In some embodiments, the extraction in saline or WFI results in a greater yield of HA or HC-HA/PTX3 complex than extraction using a different excipient, for example structured water. In some embodiments, the extract contains greater than about 900 μg/g HA to extract (wet). In some embodiments, the extract contains greater than about 1000 μg/g HA to extract (wet). In some embodiments, the extract contains greater than about 1100 μg/g HA to extract (wet). In some embodiments, the extract contains greater than about 1200 μg/g HA to extract (wet).

### Dilution

Described herein, in certain embodiments, are methods for processing a fetal support tissue product, wherein the methods comprise a dilution step. In some embodiments, the dilution step is performed on the fetal support tissue after it has been subject to the extraction and centrifugation steps. In some embodiments, the dilution step is performed on the fetal support tissue after it has been subject to the extraction and centrifugation step and before it is filtered by sterilization. In some embodiments, diluting the extract (e.g., reducing the concentration of proteins of interest in the extract) is achieved by mixing the extract with an excipient. In some embodiments, the excipient is saline, water, structured water, water for injection (WFI), or a combination thereof. In some embodiments, the excipient is WFI. In some embodiments, the extract is mixed with the excipient at a dilution factor of about 1 to 1.5, about 1.5 to 2, about 2 to 2.5, about 2.5 to 3, about 3-3.5 about 3.5 to 4, about 4 to 5, about 5 to 6, about 6 to 7, about 7 to 8, about 8 to 9, or about 9 to 10. In some embodiments, the dilution factor is greater than 5. In some embodiments, the dilution factor is greater than 10. In some embodiments, the dilution factor is about 2. In some embodiments, the excipient is WFI and the dilution factor is 2. In some embodiments, the excipient is saline and the dilution factor is 2. In some embodiments, methods described herein further comprise mixing the extract and the excipient for about 10 to 30 minutes, about 30 minutes to 1 hour, or for about 1 hour to 2 hours. In some embodiments the extract and excipient are mixed for about 30 minutes. In some embodiments, the extract and excipient are mixed at a speed of about 5-10 rpm, 10-20 rpm, 20-30 rpm, 30-40 rpm, or 40-50 rpm. In some embodiments, the extract and excipient are mixed at a speed of about 20 rpm. In some embodiments, the extract and excipient are mixed at a speed of about 20 rpm for about 1 hour. In some embodiments, the extract and excipient are mixed at a temperature of about 4° C. In some embodiments, the extract and excipient are mixed at a speed of about 20 rpm for about 1 hour at about 4° C. In some embodiments, the extract is diluted prior to a filtration step. In some embodiments, the extract is diluted after a filtration step.

In some embodiments, dilution increases the speed of filtration or the recovery rate of one or more proteins of interest after filtration, the recovery rate of one or more proteins of interest, or the potency of the extract (e.g., as measured by an ODI-TRAP assay, an M2 assay, an NO assay and/or a WST-1 assay), or a combination thereof, as compared to an undiluted extract comprising the same fetal support tissue and excipient. In some embodiments, diluted fetal support tissue comprises from about 1 μg/ml—to about 150 μg/ml of Hyaluronan (HA). In some embodiments, diluted fetal support tissue comprises from about 1 μg/ml—to about 90 μg/ml of Hyaluronan (HA). In some embodiments, diluted fetal support tissue comprises from about 90 μg/ml— to about 150 μg/ml of Hyaluronan (HA).

### Pooling of Multiple Donors

Provided herein, in certain embodiments, are methods of producing a pooled fetal support tissue product. In some instances, the methods of producing the pooled fetal support tissue product comprise any method of producing a fetal support tissue product described herein and a pooling step. In some instances, the pooling step comprises pooling fetal support tissues derived from multiple subjects. In some instances, the pooling step comprises pooling fetal support tissue products derived from multiple subjects to produce a pooled composition (e.g., a pooled drug substance). In some instances the fetal support tissue products are produced by methods described herein, which may comprises (a) cryopulverizing a fetal support tissue to generate a cryopulverized fetal support tissue; (b) extracting the cryopulverized fetal support tissue in an excipient to generate an extract; and (c) sterilizing by filtration the extract to produce the fetal support tissue product. In some instances, the pooling step comprises pooling fetal support tissue products derived from multiple donor lots to produce a pooled composition (e.g., a pooled drug substance). In some instances, a donor lot is an incoming placenta from a single subject. In some instances, the pooling step comprises pooling fetal support tissues (e.g., fetal support tissue products) from (e.g., derived from) at least 15 subjects to produce the pooled composition. In some instances, the pooling step comprises pooling fetal support tissues (e.g., fetal support tissue products) from at least 30 subjects to produce the pooled composition. In some instances, the pooling step comprises pooling fetal support tissues (e.g., fetal support tissue products) from at least 45 subjects to produce the pooled composition. In some instances, the pooling step comprises pooling fetal support tissues (e.g., fetal support tissue products) from at least 10, 15, 20, 25, 30, 35, 40, 45, 50, 55, 60, 65, 70, 75, 80, 85, 90, 95, 100, or more than 100 subjects to produce the pooled composition. In some instances, the pooling step comprises pooling fetal support tissues (e.g., fetal support tissue products) from at most 5 subjects. In some instances, the pooling step comprises pooling fetal support tissues (e.g., fetal support tissue products) from at most 9, 8, 7, 6, 5, 4, 3, or 2 subjects. In some instances, the pooling step comprises pooling multiple batches of a fetal support tissue product to produce a pooled composition. In some cases, the pooling step comprises pooling 2, 3, 4, 5, 6, 7, 8, 9, 10, 11, 12, 13, 14, or 15 batches. In some cases, no more than three batches are pooled to produce the pooled composition. In some cases, a batch is a fetal support tissue composition pooled from fetal support tissue products derived from multiple subjects. In some instances, a batch is comprises fetal support tissue products derived from 15 subjects. In some cases, a batch is pooled from at least 10, 15, 20, 25, 30, 35, 40, 45, 50, 55, 60, 65, 70, 75, 80, 85, 90, 95, 100, or more than 100 subjects. In some instances, a batch is pooled from at most 9, 8, 7, 6, 5, 4, 3, or 2 subjects. In some instances, pooling comprises pooling three batches, and each batch comprises fetal support tissue from 15 subjects.

In some cases, the pooling step comprises shaking or mixing fetal support tissues (e.g., fetal support tissue products) from multiple donors. In some cases, the mixing occurs in a container. In some cases, a shaker is used to mix the container. In some cases, the container rotates at a range of speed from about 1-40 RPM, 40-400 RPM, or 400-800 RPM. In some cases, the shaker rotates at a range of speed from about 0-40 RPM, about 40-80 RPM, about 80-120 RPM, about 120-160 RPM, about 160-200 RPM, about 200-240 RPM, about 240-280 RPM, about 2800-320 RPM, about 320-360 RPM, about 360-400 RPM, about 4000-440 RPM, about 440-480 RPM, about 480-520 RPM, about 520-560 RPM, about 560-600 RPM, about 600-640 RPM, about 640-680 RPM, about 680-720 RPM, about 720-760 RPM, or about 760-800 RPM. In some cases, the container rotates at a range of speed from about 40-400 RPM. In some cases, the mixing occurs for about 0-5 minutes, 5-10 minutes, 10-15 minutes, 15-20 minutes, 20-25 minutes, 25-30 minutes, 30-35 minutes, 35-40 minutes, 40-45 minutes, 45-50 minutes, 50-55 minutes, or 55-60 minutes. In some cases, the mixing occurs for about 1 minutes, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, or 20 minutes. In some embodiments, the mixing occurs for about 15 minutes. In some cases, the mixing occurs at a temperature of about 0° C., 1° C., 2° C., 3° C., 4° C., 5° C., 6° C., 7° C., 8° C., 9° C., 10° C., 11° C., 12° C., 13° C., 14° C. or 15° C. In some cases, the mixing occurs at a temperature of about 4° C. In some cases, the mixing occurs at a temperature of between 0° C.-5° C. or 5° C.-10° C. In some cases, the mixing is in a refrigerator. In some cases, multiple donors, batches, lots, or a combination thereof, are pooled together by mixing in a container with a shaker which rotates at a range of speed from about 40-400 RPM for about 15 minutes at a temperature of about 4° C.

In some instances, the pooling step comprises a sieving step. In some cases, the sieving step controls the maximum particle size of a pooled composition (e.g., a bulk drug substance). In some cases, the sieve has an average pore size of about 0.1-0.2 μm or less, 0.2-0.3 μm or less, 0.3-0.4 μm or less, 0.4-0.5 μm or less, 0.5-0.6 μm or less, 0.6-0.7 μm or less, 0.7-0.8 μm or less, 0.8-0.9 μm or less, 0.9-1 μm or less, 1-2 μm or less, 2-3 μm or less, 3-4 μm or less, 4-5 μm or less, 5-10 μm or less, 10-20 μm or less, 20-30 μm or less, 30-40 μm or less, 40-50 μm or less, 50-100 μm or less, 100-150 μm or less, 150-200 μm or less, 200-250 μm or less, or 250-300 μm or less. In some instances, the pooling step, comprises viral testing a batch, lot, or fetal support tissue product to pooling the batch, lot, or fetal support tissue product. In some instances, the pooling step produces a composition with a high yield of HC-HA/PTX3, HA, and other proteins of interest, improved stability, reduced variability, improved potency, or a combination thereof, as compared to a composition produced by the same method, but without being subject to a pooling step.

### Sterilization

In some embodiments, the fetal support tissue is subject to sterilization by any suitable method. Fetal support tissue products, in some embodiments, are sterilized by irradiation, by exposure to chemical sterilants, by heat, by filtration, by exposure to ethylene oxide gas, or by any process which makes the fetal support tissue product free of contamination by living microorganisms.

In some embodiments, the fetal support tissue is sterilized by filtration. In some embodiments, sterilizing the fetal support tissue by filtration comprises passing the fetal support tissue through a filter. In some embodiments, the filter pore size is selected to prevent bacteria, yeasts, molds, or viruses from passing through the filter. In some embodiments, the filter comprises pores having an average size of about 0.1-0.2 μm or less, 0.2-0.3 μm or less, 0.3-0.4 μm or less, 0.4-0.5 μm or less, 0.5-0.6 μm or less, 0.6-0.7 μm or less, 0.7-0.8 μm or less, 0.8-0.9 μm or less, 0.9-1 μm or less, 1-2 μm or less, 2-3 μm or less, 3-4 μm or less, 4-5 μm or less, 5-10 μm or less, 10-20 μm or less, 20-30 μm or less, 30-40 μm or less, 40-50 μm or less, 50-100 μm or less, 100-150 μm or less, 150-200 μm or less, 200-250 μm or less, or 250-300 μm or less. In some embodiments, the filter has an average pore size of about 0.05-0.2 μm. In some embodiments, the filter comprises pores having an average size of about 0.4 μm or less. In some embodiments, the filter comprises pores having an average size of about 0.3 μm or less. In some embodiments, the filter comprises pores having an average size of about 0.2 μm or less. In some embodiments, the filter comprises pores having an average size of about 0.2 μm. In some embodiments, sterilization by filtration comprises passing the fetal support tissue through a first filter and a second filter. In some embodiments the first filter comprises and average pore size that is larger than the average pore size of the second filter. In some embodiments, either the first or second filter have an average pore size of about 0.1-0.2 μm or less, 0.2-0.3 μm or less, 0.3-0.4 μm or less, 0.4-0.5 μm or less, 0.5-0.6 μm or less, 0.6-0.7 μm or less, 0.7-0.8 μm or less, 0.8-0.9 μm or less, 0.9-1 μm or less, 1-2 μm or less, 2-3 μm or less, 3-4 μm or less, 4-5 μm or less, 5-10 μm or less. In some embodiments each the first and second filters have an average pore size of about 0.05-0.2 μm. In some embodiments, the first filter has an average pore size of about 0.6 μm or less and the second filter has an average pore size of about 0.4 μm or less. In some embodiments, the first filter has an average pore size of about 0.5 μm or less and the second filter has an average pore size of about 0.3 μm or less. In some embodiments, the first filter has an average pore size of about 0.45 μm or less and the second filter has an average pore size of about 0.2 μm or less. In some embodiments, the first filter has an average pore size of about 0.45 μm and the second filter has an average pore size of about 0.2 μm.

In some embodiments, the any of the filters described herein is housed in a sterilization unit. In some embodiments, any of the filters described herein are membranes comprising polyethersulfone (PES), polyvinylidene fluoride (PVDF), polytetrafluorethylene (PTFE), polypropylene, polyethylene, polyamide, cellulose, cellulose nitrate, nylon, or a combination thereof. In some embodiments, any of the filters described herein have been sterilized by gamma irradiation. In some embodiments, the filtration pressure during filtration is about 0-10 psi, about 10-20 psi, about 20-30 psi, about 30-40 psi, about 40-50 psi, about 50-60 psi, about 60-70 psi, about 70-80 psi, about 80-90 psi or about 90-100 psi. In some embodiments, the effective filtration area of any of the filters described herein is from about 0-20 cm², about 20-40 cm², about 40-60 cm², about 60-80 cm², about 80-100 cm², about 100-120 cm², about 120-140 cm², about 140-160 cm², about 160-180 cm², or about 180-200 cm². In some embodiments, the overall diameter of the filter is about 0-10 mm, about 20-20 mm, about 20-30 mm, about 30-40 mm, about 40-50 mm, about 50-60 mm, about 60-70 mm, about 70-80 mm, about 80-90 mm, about 90-100 mm, about 100-200 mm, about 200-300 mm, about 300-400 mm, about 400-500 mm, about 500-600 mm, about 600-700 mm, about 700-800 mm, about 800-900 mm, or about 900-1000 mm. In some embodiments, the overall diameter of the filter is about 67 mm. In some embodiments, the overall diameter of the filter is about 68 mm. In some embodiments, the overall height of the filter is about 0-10 mm, about 20-20 mm, about 20-30 mm, about 30-40 mm, about 40-50 mm, about 50-60 mm, about 60-70 mm, about 70-80 mm, about 80-90 mm, about 90-100 mm, about 100-200 mm, about 200-300 mm, about 300-400 mm, about 400-500 mm, about 500-600 mm, about 600-700 mm, about 700-800 mm, about 800-900 mm, or about 900-1000 mm. In some embodiments, the overall height of the filter is about 82 mm. In some embodiments, the overall height of the filter is about 83 mm. In some embodiments, the filter has successfully passed a manufacturing forward flow test. In some embodiments, the forward flow rate limit for the filter is about 0.1, 0.2, 0.3, 0.4, 0.5, 0.6, 0.7, 0.8, 0.9, 1.0, or more than 1.0 mL/minute. In some embodiments, the forward flow rate limit for the filter is about 0.3, 0.4, 0.6, 0.7, 0.8, 0.9, 1.0, or more than 1.0 mL/minute at a test pressure of about 2400, 2450, 2500, 2550, 2600, 2650, 2700, 2750, 2800, 2850, 2900, or more than 2900 mbar when fully wetted with water. In some embodiments, the forward flow limit for the filter is about 0.58 mL/minute, at a test pressure of about 2760 mbar when fully wetted with water. In some embodiments, the forward flow limit for the filter is about 0.40-0.50 mL/minute, 0.50-0.60 mL/minute, or 0.60-0.70 mL/minute at a test pressure of about 2700-2800 mbar when fully wetted with water. In some embodiments, the forward flow test limit has been validated for bacterial removal by correlation of the forward flow limit with a microbiological challenge test. In some embodiments, the fetal support tissue product is test for retention of an acceptable challenge microorganism to validate the bacterial retention of the filter using procedures in conformance with the applicable Food and Drug Administration guidelines.

In some embodiments, a fetal support tissue powder product disclosed herein is subject to terminal sterilization by any suitable (e.g., medically acceptable) method. In some embodiments, a fetal support tissue powder product is disclosed herein is exposed to gamma radiation for a period of time sufficient to sterilize the fetal support tissue powder product disclosed herein.

In some embodiments, a fetal support tissue powder product disclosed herein is exposed to gamma radiation at about 10 to about 75 kilogray (kGy) for a period of time sufficient to sterilize the fetal support tissue powder product. In some embodiments, a fetal support tissue powder product disclosed herein is exposed to gamma radiation at about 10 to about 30 kGy for a period of time sufficient to sterilize the fetal support tissue product. In some embodiments, a fetal support tissue powder product disclosed herein is exposed to gamma radiation at about 15 to about 30 kGy for a period of time sufficient to sterilize the fetal support tissue product. In some embodiments, a fetal support tissue powder product disclosed herein is exposed to gamma radiation at about 25 kGy for a period of time sufficient to sterilize the fetal support tissue product. In some embodiments, a fetal support tissue powder product disclosed herein is exposed to gamma radiation at about 17.5 kGy for a period of time sufficient to sterilize the fetal support tissue powder product.

In some embodiments the fetal support tissue powder product disclosed herein is subject to electron beam (E-Beam) sterilization. In some embodiments, a fetal support tissue product disclosed herein is exposed to E-Beam radiation at about 10 to about 75 kilogray for a period of time sufficient to sterilize the fetal support tissue product. In some embodiments, a fetal support tissue product disclosed herein is exposed to E-Beam radiation at about 10 to about 30 kGy for a period of time sufficient to sterilize the fetal support tissue product. In some embodiments, a fetal support tissue product disclosed herein is exposed to E-Beam radiation at about 15 to about kGy for a period of time sufficient to sterilize the fetal support tissue product. In some embodiments, a fetal support tissue product disclosed herein is exposed to E-Beam radiation at about 25 kGy for a period of time sufficient to sterilize the fetal support tissue product. In some embodiments, a fetal support tissue product disclosed herein is exposed to E-Beam radiation at about 17.5 kGy for a period of time sufficient to sterilize the fetal support tissue product.

In some embodiments, a fetal support tissue powder product disclosed herein is exposed to an electron beam for a period of time sufficient to sterilize the fetal support tissue powder product. In some embodiments, a fetal support tissue powder product disclosed herein is exposed to X-ray radiation for a period of time sufficient to sterilize the fetal support tissue powder product. In some embodiments, a fetal support tissue powder product disclosed herein is exposed to UV radiation for a period of time sufficient to sterilize the fetal support tissue powder product.

Provided herein, in certain embodiments, are methods for preparing a fetal support tissue product, wherein the methods result in an improved percentage of HA recovered. In some embodiments, at least or about 75% 80%, 85%, 90%, 95%, 99%, or more than 99% of the HA is recovered. In some embodiments, the HA is HMW HA. In some embodiments, the methods result in an improved percentage of HC-HA/PTX3. In some embodiments, at least or about 75% 80%, 85%, 90%, 95%, 99%, or more than 99% of the HC-HA/PTX3 is recovered.

Methods as described herein, in certain embodiments, result in removal of particulates or degradants. In some embodiments, the methods described herein result in at least or about 75% 80%, 85%, 90%, 95%, 99%, or more than 99% of the particulates or degradants removed. In some embodiments, the particulates or degradants comprise chloride.

### Filling and Sealing

Described herein, in certain embodiments, are methods for processing a fetal support tissue product, wherein the methods comprise a filling step, a sealing step, or a combination thereof. In some cases, the filling step comprises filing a fetal support tissue into a container (e.g., a vial, a sealable bag, a sealable packet, a sealable pouch, etc.). In some cases, the filling step comprises filing a fetal support tissue product or a pooled bulk drug substance into a container (e.g., a vial, a sealable bag, a sealable packet, a sealable pouch, etc.). In some cases, the fetal support tissue product or pooled bulk drug substance is filled into a container until a target fill weight of the container is reached. In some cases, the fetal support tissue product or pooled bulk drug substance is sterile prior to filling. In some cases, the filling and sealing are carried out aseptically (e.g., in a controlled environment in which the air supply, materials, equipment, personnel, or a combination thereof, are regulated to maintain sterility). In some cases, the container is formed, filled, and sealed without human intervention in a sterile environment. In some cases, the container is instantly molded, filled with the fetal support tissue product or pooled bulk drug substance and sealed in a single process without any external human intervention and in a sterile environment. In some cases, the sterile product is filled and sealed in a container using a blow fill seal (BFS) equipment. In some instances, the container is sterilizable (e.g., can be sterilized by autoclaving). In some cases, the methods herein further comprise autoclaving the container. In some cases, sealing comprises sealing using a heat sealer. In some cases, the container is filled to contain about 0.01 ml to 0.50 ml, 0.51 ml to 3.0 ml, 3.1 ml to 6.0 ml, 6.1 ml to 15 ml, 15 ml to 20 ml, 20 ml to 25 ml, or 25 ml to 30 ml of the fetal support tissue product or pooled bulk drug substance. In some cases, the container is filled to contain about 1 ml, about 1.5 ml, about 2 ml, about 2.5 ml, about 3 ml, about 3.5 ml, about 4 ml, about 4.5 ml, about 5 ml, about 5.5 ml, about 6 ml, about 6.5 ml, about 7 ml, about 7.5 ml, about 8 ml, about 8.5 ml, about 9 ml, about 9.5 ml, about 10 ml, about 10.5 ml, about 11 ml, about 11.5 ml, about 12 ml, about 12.5 ml, about 13 ml, about 13.5 ml, about 14 ml, about 14.5 ml, about 15 ml, about 15.5 ml, about 16 ml, about 16.5 ml, about 17 ml, about 17.5 ml, about 18 ml, about 18.5 ml, about 19 ml, about 19.5 ml, or about 20 ml of the fetal support tissue product or pooled bulk drug substance.

### Fetal Support Tissue Product Formulations

Disclosed herein, in certain embodiments, are fetal support tissue products prepared by the method comprising: (a) cryopulverizing a fetal support tissue to generate a cryopulverized fetal support tissue; (b) extracting the cryopulverized fetal support tissue in an excipient to generate an extract; and (c) sterilizing by filtration the extract using a membrane having a pore size of about 0.6 μm followed by using a membrane having a pore size of about 0.4 μm or less. In some embodiments, the fetal support tissue is placental amniotic membrane (PAM), or substantially isolated PAM, umbilical cord amniotic membrane (UCAM) or substantially isolated UCAM, chorion or substantially isolated chorion, amnion-chorion or substantially isolated amnion-chorion, placenta or substantially isolated placenta, umbilical cord or substantially isolated umbilical cord, or any combinations thereof.

Fetal support tissue products produced by the methods described herein, in certain embodiments, comprise improved stability. In some embodiments, the fetal support tissue products comprise a high percentage of HA. In some embodiments, the fetal support tissue products comprise at least or about 75% 80%, 85%, 90%, 95%, 99%, or more than 99% of the HA. In some embodiments, the HA is HMW HA. In some embodiments, the fetal support tissue products comprise a higher percentage of HC-HA/PTX3. In some embodiments, the fetal support tissue products comprise at least or about 75% 80%, 85%, 90%, 95%, 99%, or more than 99% of the HC-HA/PTX3.

Fetal support tissue products produced by the methods described herein, in certain embodiments, comprise substantially no particulates or degradants. In some embodiments, the fetal support tissue products comprise at most about 1%, 2%, 3%, 4%, 5%, 6%, 7%, 8%, 9%, 10%, 15%, or more than 15% of particulates or degradants. In some embodiments, the particulates or degradants comprise chloride.

In some embodiments, a fetal support tissue product disclosed herein is formulated as a solution, suspension or emulsion. In some embodiments, a fetal support tissue product disclosed herein is formulated for topical administration.

Pharmaceutical formulations disclosed herein are formulated in any suitable manner. Any suitable technique, carrier, and/or excipient is contemplated for use with a fetal support tissue product disclosed herein.

### Creams and Lotions

Disclosed herein, in certain embodiments, is a topical formulation of a fetal support tissue product disclosed herein wherein the topical formulation is in the form of a cream. In certain instances, creams are semisolid (e.g., soft solid or thick liquid) formulations that include a fetal support tissue product disclosed herein dispersed in an oil-in-water emulsion or a water-in-oil emulsion.

Disclosed herein, in certain embodiments, is a topical formulation of a fetal support tissue product disclosed herein wherein the topical formulation is in the form of a lotion. In certain instances, lotions are fluid emulsions (e.g., oil-in-water emulsions or a water-in-oil emulsion). In some embodiments, the hydrophobic component of a lotion and/or cream is derived from an animal (e.g., lanolin, cod liver oil, and ambergris), plant (e.g., safflower oil, castor oil, coconut oil, cottonseed oil, menhaden oil, palm kernel oil, palm oil, peanut oil, soybean oil, rapeseed oil, linseed oil, rice bran oil, pine oil, sesame oil, or sunflower seed oil), or petroleum (e.g., mineral oil, or petroleum jelly).

### Ointments

Disclosed herein, in certain embodiments, is a topical formulation of a fetal support tissue product disclosed herein wherein the topical formulation is in the form of an ointment. In certain instances, ointments are semisolid preparations that soften or melt at body temperature.

### Pastes

Disclosed herein, in certain embodiments, is a topical formulation of a fetal support tissue product disclosed herein wherein the topical formulation is in the form of a paste. In certain instances, pastes contain at least 20% solids. In certain instances, pastes are ointments that do not flow at body temperature.

### Gels and Films

Disclosed herein, in certain embodiments, is a topical formulation of a fetal support tissue product disclosed herein wherein the topical formulation is in the form of a gel. In certain instances, gels are semisolid (or semi-rigid) systems consisting of dispersions of large organic molecules dispersed in a liquid. In certain instances, gels are water-soluble and are removed using warm water or saline.

In certain instances, in the treatment of dermal lesions, contacting lesions with a dressing can often disturb injured tissues. The removal of many dressings for wounds such as burns surface lesions that involve a significant area of the skin can cause significant pain and often can re-open at least portions of partially healed wounds. In some instances, a topical formulation of a fetal support tissue product disclosed herein is applied as a liquid to the affected area and the liquid gels as a film on the affected area. In some instances, the film is a water soluble film and can be removed with water or a mild aqueous detergent, avoiding pain and discomfort associated with the removal of wound dressings. In certain instances, the topical formulation described herein is a dermal film comprising a flexible film made of a polyalkyloxazoline. In some instances, the film has a structural layer made of a polyalkyloxazoline and a pressure sensitive adhesive layer that keeps the film in place.

### Sticks

Disclosed herein, in certain embodiments, is a topical formulation of a fetal support tissue product disclosed herein wherein the topical formulation is in the form of a stick. In certain instances, sticks are solid dosage forms that melt at body temperature. In some embodiments, a stick comprises a wax, a polymer, a resin, dry solids fused into a firm mass, and/or fused crystals. In some embodiments, a topical formulation of a fetal support tissue product disclosed herein is in the form of a styptic pencil (i.e., a stick prepared by (1) heating crystals until they lose their water of crystallization and become molten, and (2) pouring the molten crystals into molds and allowing them to harden). In some embodiments, a topical formulation of a fetal support tissue product disclosed herein is in the form of stick wherein the stick comprises a wax (e.g., the wax is melted and poured into appropriate molds in which they solidify in stick form).

In some embodiments, a topical formulation of a fetal support tissue product disclosed herein is in the form of stick wherein the stick comprises a melting base (i.e., a base that softens at body temperature). Examples of melting bases include, but are not limited to, waxes, oils, polymers and gels. In some embodiments, a topical formulation of a fetal support tissue product disclosed herein is in the form of stick wherein the stick comprises a moisten base (i.e., a base that is activated by the addition of moisture).

### Patches

Disclosed herein, in certain embodiments, is a topical formulation of a fetal support tissue product disclosed herein wherein the topical formulation is administered via a patch. In some embodiments, a topical formulation of a fetal support tissue product disclosed herein is dissolved and/or dispersed in a polymer or an adhesive. In some embodiments, a film, a patch disclosed herein is constructed for continuous, pulsatile, or on demand delivery of a fetal support tissue product.

### Wound Dressings

Disclosed herein, in certain embodiments, is a topical formulation of a fetal support tissue product disclosed herein wherein the topical formulation is administered with (or via) a wound dressing. Wound dressings include, but are not limited to gauzes, transparent film dressings, hydrogels, polyurethane foam dressings, hydrocolloids and alginates. In certain instances, wound dressings promote wound healing. In some instances, wound dressings reduce or inhibit aberrant wound healing.

### Implants/Prosthesis

Disclosed herein, in certain embodiments, is an implant or prosthesis comprising a fetal support tissue product disclosed herein. In some embodiments, the prosthesis is an artificial joint. In some embodiments, the implant is a stent.

In some embodiments, the prosthesis is an artificial hip joint. In some embodiments, the fetal support tissue product is coated onto the outside of the artificial hip joint. In some embodiments, the fetal support tissue product elutes from the artificial hip into the surrounding tissue.

In some embodiments, the prosthesis is an artificial knee. In some embodiments, the fetal support tissue product is coated onto the outside of the artificial knee. In some embodiments, the fetal support tissue product elutes from the artificial knee into the surrounding tissue.

In some embodiments, the prosthesis is an artificial glenohumeral joint. In some embodiments, the fetal support tissue product is coated onto the outside of the artificial glenohumeral joint. In some embodiments, the fetal support tissue product elutes from the artificial glenohumeral joint into the surrounding tissue.

In some embodiments, the prosthesis is an artificial ankle. In some embodiments, the fetal support tissue product is coated onto the outside of the artificial ankle. In some embodiments, the fetal support tissue product elutes from the artificial ankle into the surrounding tissue.

In some embodiments, the implant is a coronary stent. In some embodiments, the fetal support tissue product is coated onto the outside of the stent. In some embodiments, the fetal support tissue product elutes from the stent into the surrounding cardiac tissue. In some embodiments, the bone stent is placed in a bone fracture. In some embodiments, the bone stent is expandable or contractible.

In some embodiments, the implant is a ureteral stent. In some embodiments, the fetal support tissue product is coated onto the outside of the stent. In some embodiments, the fetal support tissue product elutes from the stent into the surrounding tissue. In some embodiments, the bone stent is placed in a bone fracture. In some embodiments, the bone stent is expandable or contractible.

In some embodiments, the implant is a urethral or prostatic stent. In some embodiments, the fetal support tissue product is coated onto the outside of the stent. In some embodiments, the fetal support tissue product elutes from the stent into the surrounding tissue. In some embodiments, the bone stent is placed in a bone fracture. In some embodiments, the bone stent is expandable or contractible.

In some embodiments, the implant is an esophageal stent. In some embodiments, the fetal support tissue product is coated onto the outside of the stent. In some embodiments, the fetal support tissue product elutes from the stent into the surrounding tissue. In some embodiments, the bone stent is placed in a bone fracture. In some embodiments, the bone stent is expandable or contractible.

In some embodiments, the implant is a bone implant. In some embodiments, the bone implant is an osseointegrated implant. As used herein, an “osseointegrated implant” means a three dimensional implant containing pores into which osteoblasts and supporting connective tissue can migrate. In some embodiments, the bone implant comprises a composition described herein. In some embodiments, the bone implant is a dental implant. In some embodiments, the bone implant is used for knee or joint replacement. In some embodiments, the bone implant is a craniofacial prosthesis (e.g., an artificial ear, orbital prosthesis, nose prosthesis).

In some embodiments, the implant is a bone stent. In some embodiments, the fetal support tissue product is coated onto the outside of the stent. In some embodiments, the fetal support tissue product elutes from the stent into the surrounding bone. In some embodiments, the bone stents are inserted into the intramedullary canal of a bone. In some embodiments, the bone stent is placed in the sinus tarsi. In some embodiments, the bone stent in placed in a knee or joint. In some embodiments, the bone stent is placed in a bone fracture. In some embodiments, the bone stent is expandable or contractible.

In some embodiments, the implant is a K-wire or Denham pin. In some embodiments, the fetal support tissue product is coated onto the outside of the K-wire or Denham pin. In some embodiments, the fetal support tissue product elutes from the K-wire or Denham pin into the surrounding bone.

### Miscellaneous Formulations

In some embodiments, a fetal support tissue product disclosed herein is administered as a dermal paint. As used herein, paints (also known as film formers) are solutions comprised of a solvent, a monomer or polymer, an active agent, and optionally one or more pharmaceutically-acceptable excipients. After application to a tissue, the solvent evaporates leaving behind a thin coating comprised of the monomers or polymers, and the active agent. The coating protects active agents and maintains them in an immobilized state at the site of application. This decreases the amount of active agent which may be lost and correspondingly increases the amount delivered to the affected area of the skin of an individual. By way of non-limiting example, paints include collodions (e.g. Flexible Collodion, USP), and solutions comprising saccharide siloxane copolymers and a cross-linking agent. Collodions are ethyl ether/ethanol solutions containing pyroxylin (a nitrocellulose). After application, the ethyl ether/ethanol solution evaporates leaving behind a thin film of pyroxylin. In solutions comprising saccharide siloxane copolymers, the saccharide siloxane copolymers form the coating after evaporation of the solvent initiates the cross-linking of the saccharide siloxane copolymers.

In certain embodiments, a fetal support tissue product described herein is optionally incorporated within controlled release particles, lipid complexes, liposomes, nanoparticles, microspheres, microparticles, nanocapsules or other agents which enhance or facilitate localized delivery to the skin. An example of a conventional microencapsulation process for pharmaceutical preparations is shown in U.S. Pat. No. 3,737,337, incorporated herein by reference for such disclosure.

In some instances, a fetal support tissue product described herein is a liposomal formulation. Liposomes are prepared by introducing an aqueous buffer into a mixture of phospholipid and organic solvent and the organic solvent is subsequently removed by evaporation under reduced pressure. An example of a liposomal preparation is described in Proc. Natl. Acad. Sci. 1978, 75, 4194-98, incorporated herein by reference for such disclosure. Liposomes are fractionated according to their particle sizes by size exclusion chromatography (SEC). The subfractions of liposomes are further sized by photon correlation spectroscopy (PCS) for their particle sizes. Enzymatic assays (e.g., phosphatidylcholine (PC) assay) are used to analyze lipid contents of liposomes.

### Dermatological Excipients

Disclosed herein, in certain embodiments, are formulations of a fetal support tissue product disclosed herein wherein the formulations comprise a carrier. Suitable carriers include, but are not limited to, carbomer, cellulose, collagen, ethanol, glycerin, hexylene glycol, hyaluronic acid, hydroxypropyl cellulose, phosphoric acid, polyols (propyleneglycol, polyethylene-glycol, glycerol, cremophor and the like), polysorbate 80, saline, sodium hydroxide, sodium phosphate, sorbital, water, xanthan gum vegetable oils (such as olive oil), injectable organic esters (e.g., ethyl oleate), fatty oils (e.g., sesame oil), and synthetic fatty acid esters (e.g., ethyl oleate or triglycerides).

### Penetration Enhancers

Disclosed herein, in certain embodiments, are formulations of a fetal support tissue product disclosed herein wherein the formulations comprise a penetration enhancer. Penetration enhancers include, but are not limited to, sodium lauryl sulfate, sodium laurate, polyoxyethylene-20-cetyl ether, laureth-9, sodium dodecylsulfate, dioctyl sodium sulfosuccinate, polyoxyethylene-9-lauryl ether (PLE), Tween 80, nonylphenoxypolyethylene (NP-POE), polysorbates, sodium glycocholate, sodium deoxycholate, sodium taurocholate, sodium taurodihydrofusidate, sodium glycodihydrofusidate, oleic acid, caprylic acid, mono- and di-glycerides, lauric acids, acylcholines, caprylic acids, acylcarnitines, sodium caprates, EDTA, citric acid, salicylates, DMSO, decylmethyl sulfoxide, ethanol, isopropanol, propylene glycol, polyethylene glycol, glycerol, propanediol, and diethylene glycol monoethyl ether. In certain embodiments, the topical formulations described herein are designed for minimal systemic exposure and include, for example, low amounts of penetration enhancers.

### Gelling Agents

Disclosed herein, in certain embodiments, are formulations of a fetal support tissue product disclosed herein wherein the formulations comprise a gelling (or thickening) agent. In some embodiments, a formulation disclosed herein further comprises from about 0.1% to about 5%, from about 0.1% to about 3%, or from about 0.25% to about 2%, of a gelling agent. In certain embodiments, the viscosity of a formulation disclosed herein is in the range from about 100 to about 500,000 cP, about 100 cP to about 1,000 cP, about 500 cP to about 1500 cP, about 1000 cP to about 3000 cP, about 2000 cP to about 8,000 cP, about 4,000 cP to about 10,000 cP, about 10,000 cP to about 50,000 cP. Suitable gelling agents for use in preparation of the gel formulation include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum, acacia (gum arabic), agar, aluminum magnesium silicate, sodium alginate, sodium stearate, bladderwrack, bentonite, carbomer, carrageenan, carbopol, xanthan, cellulose, microcrystalline cellulose (MCC), ceratonia, chondrus, dextrose, furcellaran, gelatin, ghatti gum, guar gum, hectorite, lactose, sucrose, maltodextrin, mannitol, sorbitol, honey, maize starch, wheat starch, rice starch, potato starch, gelatin, sterculia gum, polyethylene glycol (e.g. PEG 200-4500), gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), or combinations thereof.

Gels include a single-phase or a two-phase system. A single-phase gel consists of organic macromolecules distributed uniformly throughout a liquid in such a manner that no apparent boundaries exist between the dispersed macromolecules and the liquid. Some single-phase gels are prepared from synthetic macromolecules (e.g., carbomer) or from natural gums, (e.g., tragacanth). In some embodiments, single-phase gels are generally aqueous, but will also be made using alcohols and oils. Two-phase gels consist of a network of small discrete particles.

Gels can also be classified as being hydrophobic or hydrophilic. In certain embodiments, the base of a hydrophobic gel consists of a liquid paraffin with polyethylene or fatty oils gelled with colloidal silica, or aluminum or zinc soaps. In contrast, the base of hydrophobic gels usually consists of water, glycerol, or propylene glycol gelled with a suitable gelling agent (e.g., tragacanth, starch, cellulose derivatives, carboxyvinylpolymers, and magnesium-aluminum silicates).

Suitable agents for use in formulations that are applied as liquids and gel upon application to the skin into a film include but are not limited to polymers composed of polyoxypropylene and polyoxyethylene that are known to form thermoreversible gels when incorporated into aqueous solutions. These polymers have the ability to change from the liquid state to the gel state at temperatures close to body temperature, therefore allowing useful formulations that are applied as gels and/or films to the affected area. Examples of polymers that gel at body temperature and are used in gels and/or films described herein include and are not limited to poloxamers (e.g., PLURONICS F68®, F88®, F108®, and F127®, which are block copolymers of ethylene oxide and propylene oxide). The liquid state-to-gel state phase transition is dependent on the polymer concentration and the ingredients in the solution.

### Adhesives

In some instances, a formulation described herein comprises pressure sensitive adhesives (e.g., polyalkyloxazoline polymers) and allows for application of an adhesive film to an affected area of skin.

### Emollients

Disclosed herein, in certain embodiments, are formulations of a fetal support tissue product disclosed herein wherein the formulations comprise an emollient. Emollients include, but are not limited to, castor oil esters, cocoa butter esters, safflower oil esters, cottonseed oil esters, corn oil esters, olive oil esters, cod liver oil esters, almond oil esters, avocado oil esters, palm oil esters, sesame oil esters, squalene esters, kikui oil esters, soybean oil esters, acetylated monoglycerides, ethoxylated glyceryl monostearate, hexyl laurate, isohexyl laurate, isohexyl palmitate, isopropyl palmitate, methyl palmitate, decyloleate, isodecyl oleate, hexadecyl stearate decyl stearate, isopropyl isostearate, methyl isostearate, diisopropyl adipate, diisohexyl adipate, dihexyldecyl adipate, diisopropyl sebacate, lauryl lactate, myristyl lactate, and cetyl lactate, oleyl myristate, oleyl stearate, and oleyl oleate, pelargonic acid, lauric acid, myristic acid, palmitic acid, stearic acid, isostearic acid, hydroxystearic acid, oleic acid, linoleic acid, ricinoleic acid, arachidic acid, behenic acid, erucic acid, lauryl alcohol, myristyl alcohol, cetyl alcohol, hexadecyl alcohol, stearyl alcohol, isostearyl alcohol, hydroxystearyl alcohol, oleyl alcohol, ricinoleyl alcohol, behenyl alcohol, erucyl alcohol, 2-octyl dodecanyl alcohol, lanolin and lanolin derivatives, beeswax, spermaceti, myristyl myristate, stearyl stearate, carnauba wax, candelilla wax, lecithin, and cholesterol.

### Miscellaneous Excipients

In certain embodiments, a formulation comprising a fetal support tissue product disclosed herein comprises additional excipients such as, by way of example, abrasives, absorbents, anticaking agents, astringents, essential oils, fragrances, skin-conditioning agents, skin healing agents, skin protectants (e.g., sunscreens, or ultraviolet light absorbers or scattering agents), skin soothing agents, or combinations thereof.

### Methods of Use

Disclosed herein, in certain embodiments, are methods of using the fetal support tissue product produced by the methods described herein. In some embodiments, the fetal support tissue is placental amniotic membrane (PAM), or substantially isolated PAM, umbilical cord amniotic membrane (UCAM) or substantially isolated UCAM, chorion or substantially isolated chorion, amnion-chorion or substantially isolated amnion-chorion, placenta or substantially isolated placenta, umbilical cord or substantially isolated umbilical cord, or any combinations thereof. In some embodiments, the fetal support tissue products produced by the methods disclosed herein comprise the fetal support tissue and a pharmaceutically acceptable carrier. In some embodiments, the fetal support tissue products disclosed herein are formulated for administration by topical administration or injection. In some embodiments, the fetal support tissue products disclosed herein are formulated as a solution, suspension or emulsion.

In some embodiments, a fetal support tissue product disclosed herein is used to inhibit at least one of the following: scarring, inflammation, adhesion and angiogenesis. In some embodiments, a fetal support tissue product disclosed herein is used to promote wound healing. In some embodiments, the use is a homologous use. In some embodiments, a fetal support tissue product disclosed herein is minimally manipulated. In some embodiments, a fetal support tissue product disclosed herein does not comprise another article, except for water, crystalloids, or a sterilizing, preserving, or storage agent. In some embodiments, a fetal support tissue product disclosed herein does not have a systemic effect and is not dependent upon the metabolic activity of living cells for its primary function.

In some embodiments, a fetal support tissue product disclosed herein is used as a covering (e.g., a wound covering). In some embodiments, the use is a homologous use. In some embodiments, the fetal support tissue product is minimally manipulated. In some embodiments, the fetal support tissue product does not comprise another article, except for water, crystalloids, or a sterilizing, preserving, or storage agent. In some embodiments, the fetal support tissue product does not have a systemic effect and is not dependent upon the metabolic activity of living cells for its primary function.

In some embodiments, a fetal support tissue product disclosed herein is used to promote wound repair. In some embodiments, the use is a homologous use. In some embodiments, the fetal support tissue product is minimally manipulated. In some embodiments, the fetal support tissue product does not comprise another article, except for water, crystalloids, or a sterilizing, preserving, or storage agent. In some embodiments, the fetal support tissue product does not have a systemic effect and is not dependent upon the metabolic activity of living cells for its primary function.

In some embodiments, a fetal support tissue product disclosed herein is used as a barrier to adhesion. In some embodiments, the use is a homologous use. In some embodiments, the fetal support tissue product is minimally manipulated. In some embodiments, the fetal support tissue product does not comprise another article, except for water, crystalloids, or a sterilizing, preserving, or storage agent. In some embodiments, the fetal support tissue product does not have a systemic effect and is not dependent upon the metabolic activity of living cells for its primary function.

In some embodiments, a fetal support tissue product disclosed herein comprises proteins, glycans, protein-glycan complexes (e.g., a complex of hyaluronic acid and a heavy chain of IαI and PTX3) and enzymes that promote tissue repair. For example, the stroma of AM contains growth factors, anti-angiogenic and anti-inflammatory proteins, as well as natural inhibitors to various proteases. In some embodiments, proteins and enzymes found in a fetal support tissue product disclosed herein diffuse out of the fetal support tissue product and into the surrounding tissue.

### Injured Tissue Repair and Supplementation

In some embodiments, a fetal support tissue product disclosed herein is used as a wound covering or is used to facilitate wound repair. In some embodiments, the use is a homologous use (e.g., a functional homologous use or a structural homologous use). In some embodiments, the fetal support tissue product is minimally manipulated. In some embodiments, the fetal support tissue product does not comprise another article, except for water, crystalloids, or a sterilizing, preserving, or storage agent. In some embodiments, the fetal support tissue product does not have a systemic effect and is not dependent upon the metabolic activity of living cells for its primary function.

In some embodiments, the tissue was damaged, compromised, or lost due to an injury (e.g., a burn; a surgical incision; an area of necrosis resulting from an infection, trauma, or a toxin; a laceration). In some embodiments, the tissue was damaged, compromised, or lost due to a burn. In some embodiments, the tissue was damaged, compromised, or lost due to a wound (e.g., an incision, laceration, abrasion). In some embodiments, the tissue was damaged, compromised, or lost due to necrosis. In some embodiments, the tissue was damaged, compromised, or lost due to ulceration. In some embodiments, the fetal support tissue product is administered by parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the fetal support tissue product is administered epidurally, intrathecally, through inhalation, intravenously, or a combination thereof.

In some embodiments, a fetal support tissue product disclosed herein comprises proteins, glycans, protein-glycan complexes (e.g., a complex of hyaluronic acid and a heavy chain of IαI and PTX3) and enzymes that promote tissue repair. For example, the stroma of AM contains growth factors, anti-angiogenic and anti-inflammatory proteins, as well as natural inhibitors to various proteases. In some embodiments, proteins and enzymes found in a fetal support tissue product disclosed herein diffuse out of the fetal support tissue product and into the surrounding tissue.

### Burns

In some embodiments, a fetal support tissue product disclosed herein is applied to a burn. In some embodiments, a fetal support tissue product disclosed herein is applied to a first degree burn. In some embodiments, a fetal support tissue product disclosed herein is applied to a second degree burn. In some embodiments, a fetal support tissue product disclosed herein is applied to a third degree burn. In some embodiments, the fetal support tissue product is applied to a substrate prior to be placed on the burn.

### Wounds

In some embodiments, a fetal support tissue product disclosed herein is applied to a wound in the skin (e.g., an incision, laceration, abrasion, ulcer, puncture, penetration). In some embodiments, the fetal support tissue product is applied to a substrate prior to being placed on the wound. In some embodiments, the fetal support tissue product is administered by parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the fetal support tissue product is administered epidurally, intrathecally, through inhalation, intravenously, or a combination thereof.

In some embodiments, a fetal support tissue product disclosed herein is applied to an incision in an organ (e.g., the skin, brain, stomach, kidneys, liver, intestines, lungs, bladder, trachea, esophagus, vagina, ureter, and blood vessel walls). In some embodiments, a fetal support tissue product disclosed herein is applied to a surgical incision. In some embodiments, a fetal support tissue product disclosed herein is applied to the site of a colon resection. In some embodiments, a fetal support tissue product disclosed herein is applied to the site of a gastrectomy. In some embodiments, a fetal support tissue product disclosed herein is applied to the site of a breast surgery (e.g., breast reduction surgery, breast augmentation surgery, and mastectomy). In some embodiments, the fetal support tissue product is applied to a substrate prior to being placed on the wound.

In some embodiments, a fetal support tissue product disclosed herein is used as a covering over an incision in the skin (e.g., an incision to the epidermis, dermis, and/or hypodermis). In some embodiments, a fetal support tissue product disclosed herein is used to repair or supplement the skin following hemorrhoid surgery. In some embodiments, the fetal support tissue product is applied to a substrate prior to being placed on the wound.

### Necrosis

In some embodiments, a fetal support tissue product disclosed herein is used as a protective graft over an area of necrotic tissue (e.g., from an infection). In some embodiments, a fetal support tissue product disclosed herein is used as a protective graft over an area of necrotic skin. In some embodiments, a fetal support tissue product disclosed herein is placed on an area of necrotic tissue. In some embodiments, the fetal support tissue product is applied to a substrate prior to being placed on the necrotic tissue. In some embodiments, the fetal support tissue product is administered by parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the fetal support tissue product is administered epidurally, intrathecally, through inhalation, intravenously, or a combination thereof.

### Ulcer

In some embodiments, a fetal support tissue product disclosed herein is used as a protective covering over an ulcer. In some embodiments, the fetal support tissue product is applied to a substrate prior to being placed on the ulcer. In some embodiments, the fetal support tissue product is administered by parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the fetal support tissue product is administered epidurally, intrathecally, through inhalation, intravenously, or a combination thereof.

In some embodiments, the ulcer is a foot ulcer (e.g., a diabetic foot ulcer or an arterial insufficiency ulcer). In some embodiments, treating a foot ulcer comprises (a) preparing the wound (e.g., debriding the wound); and (b) placing a fetal support tissue product disclosed herein on the wound. In some embodiments, treating a foot ulcer comprises (a) preparing the wound (e.g., debriding the wound); (b) placing a fetal support tissue product disclosed herein on the wound; and (c) covering the fetal support tissue product with a protective barrier (e.g., a silvercell dressing, metipel, gauze, or a bandage). In some embodiments, the fetal support tissue product is applied to a substrate prior to be placed on the ulcer.

In some embodiments, the ulcer is a venous stasis (VS) ulcer. In some embodiments, treating a VS ulcer comprises (a) preparing the wound (e.g., debriding the wound); and (b) placing A fetal support tissue product disclosed herein on the wound. In some embodiments, treating a VS ulcer comprises (a) preparing the wound (e.g., debriding the wound); (b) placing a fetal support tissue product disclosed herein on the wound; and (c) covering the fetal support tissue product with a protective barrier (e.g., a wound veil, antimicrobial dressing, gauze, or a bandage). In some embodiments, the fetal support tissue product is applied to a substrate prior to being placed on the wound.

In some embodiments, the ulcer is a corneal ulcer (i.e., ulcerative keratitis). In some embodiments, treating a corneal ulcer comprises (a) preparing the wound (e.g., debriding the wound); and (b) placing a fetal support tissue product disclosed herein on the wound. In some embodiments, treating a corneal ulcer comprises (a) preparing the wound (e.g., debriding the wound); (b) placing a fetal support tissue product disclosed herein on the wound; and (c) covering the fetal support tissue product or fetal support tissue product with a protective barrier (e.g., a contact lens or a bandage). In some embodiments, the fetal support tissue product is applied to a substrate prior to being placed on the wound.

### Soft Tissue Uses

Disclosed herein, in certain embodiments, is the use of a fetal support tissue product disclosed herein for repairing, reconstructing, replacing, or supplementing a recipient's damaged, compromised, or missing soft tissue (e.g., tendons).

In some embodiments, the use is a homologous use. In some embodiments, the fetal support tissue product is minimally manipulated. In some embodiments, the fetal support tissue product does not comprise another article, except for water, crystalloids, or a sterilizing, preserving, or storage agent. In some embodiments, the fetal support tissue product does not have a systemic effect and is not dependent upon the metabolic activity of living cells for its primary function.

In some embodiments, a fetal support tissue product disclosed herein comprises proteins, glycans, protein-glycan complexes (e.g., a complex of hyaluronic acid and a heavy chain of IαI and PTX3) and enzymes that promote tissue repair. For example, the stroma of AM contains growth factors, anti-angiogenic and anti-inflammatory proteins, as well as natural inhibitors to various proteases. In some embodiments, proteins and enzymes found in a fetal support tissue product disclosed herein diffuse out of the fetal support tissue product and into the surrounding tissue.

In some embodiments, a fetal support tissue product disclosed herein described herein is used as a covering over an incision in soft tissue (e.g., eyelids form the tissue plane between different layers of soft tissue). In some embodiments, the fetal support tissue product is applied to a substrate and then used as a covering over an incision in soft tissue (e.g., eyelids form the tissue plane between different layers of soft tissue). In some embodiments, the fetal support tissue product is administered by parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the fetal support tissue product is administered epidurally, intrathecally, through inhalation, intravenously, or a combination thereof.

In some embodiments, a fetal support tissue product disclosed herein is used as structural (tectonic) support for soft tissue.

In some embodiments, a fetal support tissue product disclosed herein prevents adhesion in joint or tendon repairs.

In some embodiments, a fetal support tissue product disclosed herein is used in the repair a tendon or joint (such as rotator cuff repairs, hand tendon repairs). In some embodiments, a fetal support tissue product disclosed herein is used to reinforce a tendon or joint. In some embodiments, a fetal support tissue product disclosed herein is used to prevent adhesion of a healing tendon to surrounding tissue, tendons or joints. In some embodiments, a fetal support tissue product disclosed herein is used to prevent the formation of scar tissue on a tendon.

In some embodiments, a fetal support tissue product disclosed herein is applied to a substrate and the substrate/fetal support tissue product is used to augment smaller tendons and ligaments of the foot and ankle, including the posterior tibial tendon, the peroneal tendons, the flexor and extensor tendons, and the ligaments of the lateral ankle complex. In some embodiments, a fetal support tissue product disclosed herein is applied to a substrate and the substrate/fetal support tissue product is used to reinforce primary repair of the quadriceps and patellar tendons surrounding the knee. In some embodiments, a fetal support tissue product disclosed herein is applied to a substrate and the substrate/fetal support tissue product is used as a periosteal patch for bone graft in joint replacement. In some embodiments, a fetal support tissue product disclosed herein is applied to a substrate and the substrate/fetal support tissue product is used to augment deficient hip and knee capsular tissue following total joint revision surgery.

In some embodiments, a fetal support tissue product disclosed herein is applied to a substrate and the substrate/fetal support tissue product is used in the repair of a torn rotator cuff. In some embodiments, a fetal support tissue product disclosed herein is applied to a substrate and the substrate/fetal support tissue product is used as a patch over a rotator cuff muscle or tendon (e.g., the supraspinatus tendon). In some embodiments, a fetal support tissue product disclosed herein is applied to a substrate and the substrate/fetal support tissue product is used to reconstruct a rotator cuff muscle or tendon (e.g., the supraspinatus tendon). In some embodiments, a fetal support tissue product disclosed herein is applied to a substrate and the substrate/fetal support tissue product is used to augment a rotator cuff muscle or tendon (e.g., the supraspinatus tendon). In some embodiments, a fetal support tissue product disclosed herein is applied to a substrate and the substrate/fetal support tissue product is used to reinforce a rotator cuff muscle or tendon (e.g., the supraspinatus tendon). In some embodiments, a fetal support tissue product disclosed herein is applied to a substrate and the substrate/fetal support tissue product is used to prevent adhesion of soft tissue to a rotator cuff muscle or tendon (e.g., the supraspinatus tendon).

In some embodiments, a fetal support tissue product disclosed herein is used in the repair gingiva. In some embodiments, a fetal support tissue product disclosed herein is used in the repair gingival recession. In some embodiments, a fetal support tissue product disclosed herein is applied to a substrate and used as a patch over gingiva. In some embodiments, a fetal support tissue product disclosed herein is applied to substrate and used as a patch over an exposed tooth root surface. In some embodiments, a fetal support tissue product disclosed herein is used to reconstruct gingiva. In some embodiments, a fetal support tissue product disclosed herein is used to augment gingiva. In some embodiments, a fetal support tissue product disclosed herein is used to reinforce gingiva. In some embodiments, a fetal support tissue product disclosed herein is used to prevent adhesion of soft tissue to gingiva.

In some embodiments, a fetal support tissue product described herein is applied to a substrate and the substrate/fetal support tissue product is used as a protective graft over an incision or tear in the fascia. In some embodiments, a fetal support tissue product disclosed herein is applied to a substrate and the substrate/fetal support tissue product is used as structural (tectonic) support the fascia. In some embodiments, a fetal support tissue product disclosed herein is applied to a substrate and the substrate/fetal support tissue product is used as a replacement or supplement for the fascia. In some embodiments, a fetal support tissue product disclosed herein is applied to a substrate and the substrate/fetal support tissue product is used to repair a hernia (e.g., to repair the fascia). In some embodiments, a fetal support tissue product disclosed herein is applied to a substrate and the substrate/fetal support tissue product is used to repair an inguinal hernia. In some embodiments, a fetal support tissue product disclosed herein is applied to a substrate and the substrate/fetal support tissue product is used to repair a femoral hernia. In some embodiments, a fetal support tissue product disclosed herein is applied to a substrate and the substrate/fetal support tissue product is used to repair an umbilical hernia. In some embodiments, a fetal support tissue product disclosed herein is applied to a substrate and the substrate/fetal support tissue product is used to repair an incisional hernia. In some embodiments, a fetal support tissue product disclosed herein is applied to a substrate and the substrate/fetal support tissue product is used to repair a diaphragmatic hernia. In some embodiments, a fetal support tissue product disclosed herein is applied to a substrate and the substrate/fetal support tissue product is used to repair a Cooper's hernia, an epigastric hernia, an hiatal hernia, a Littre's hernia, a lumbar hernia, a maydl hernia, an obturator hernia, a pantaloon hernia, a paraesophageal hernia, a periumbilical hernia, a perineal hernia, a properitoneal hernia, a Richter's hernia, a sliding hernia, a sciatic hernia, a spigelian hernia, a sports hernia, a Velpeau hernia, or a Amyand's hernia.

In some embodiments, a fetal support tissue product disclosed herein is applied to a substrate and the substrate/fetal support tissue product is used to repair a spinal disc herniation. In some embodiments, a fetal support tissue product disclosed herein is applied to a substrate and the substrate/fetal support tissue product is used as a protective graft over an incision or tear in a spinal disc. In some embodiments, a fetal support tissue product disclosed herein is applied to a substrate and the substrate/fetal support tissue product is used as a protective graft over an incision or tear in an annulus fibrosis. In some embodiments, a fetal support tissue product disclosed herein is applied to a substrate and the substrate/fetal support tissue product is used as structural (tectonic) support a spinal disc. In some embodiments, a fetal support tissue product disclosed herein is applied to a substrate and the substrate/fetal support tissue product is used as structural (tectonic) support an annulus fibrosis. In some embodiments, a fetal support tissue product disclosed herein is applied to a substrate and the substrate/fetal support tissue product is used as a replacement or supplement for a spinal disc. In some embodiments, a fetal support tissue product disclosed herein is applied to a substrate and the substrate/fetal support tissue product is used as structural (tectonic) support a spinal disc. In some embodiments, a fetal support tissue product disclosed herein is applied to a substrate and the substrate/fetal support tissue product is used as a replacement or supplement for an annulus fibrosis.

In some embodiments, a fetal support tissue product disclosed herein is applied to a substrate and the substrate/fetal support tissue product is used over an incision in the brain, or in one (or all) of the meninges (i.e., the dura mater, the pia mater, and/or the arachnoid mater). In some embodiments, a fetal support tissue product disclosed herein is applied to a substrate and the substrate/fetal support tissue product is used as structural (tectonic) support for one (or all) of the meninges (i.e., the dura mater, the pia mater, and/or the arachnoid mater). In some embodiments, a fetal support tissue product disclosed herein is applied to a substrate and the substrate/fetal support tissue product is used as a replacement for one (or all) of the meninges (i.e., the dura mater, the pia mater, and/or the arachnoid mater).

In some embodiments, a fetal support tissue product disclosed herein is applied to a substrate and the substrate/fetal support tissue product is used over an incision in a lung or in the pleura. In some embodiments, a fetal support tissue product disclosed herein is applied to a substrate and the substrate/fetal support tissue product is used as structural (tectonic) support for the pleura. In some embodiments, a fetal support tissue product disclosed herein is applied to a substrate and the substrate/fetal support tissue product is used as a replacement for the pleura.

In some embodiments, a fetal support tissue product disclosed herein is applied to a substrate and the substrate/fetal support tissue product is used over an incision in a tympanic membrane. In some embodiments, a fetal support tissue product disclosed herein is applied to a substrate and the substrate/fetal support tissue product is used as structural (tectonic) support for a tympanic membrane. In some embodiments, a fetal support tissue product disclosed herein is applied to a substrate and the substrate/fetal support tissue product is used as a replacement for a tympanic membrane.

In some embodiments, a fetal support tissue product disclosed herein is applied to a substrate and the substrate/fetal support tissue product is used as a protective graft over an incision in the heart or the pericardium. In some embodiments, a fetal support tissue product disclosed herein is applied to a substrate and the substrate/fetal support tissue product is used as structural (tectonic) support for the pericardium. In some embodiments, a fetal support tissue product disclosed herein is applied to a substrate and the substrate/fetal support tissue product is used as a replacement for the pericardium.

In some embodiments, a fetal support tissue product disclosed herein is applied to a substrate and the substrate/fetal support tissue product is used as a protective graft over an incision in the peritoneum. In some embodiments, a fetal support tissue product disclosed herein is applied to a substrate and the substrate/fetal support tissue product is used as structural (tectonic) support for the peritoneum. In some embodiments, a fetal support tissue product disclosed herein is applied to a substrate and the substrate/fetal support tissue product is used as a replacement for the peritoneum.

### Ophthalmic Uses

Disclosed herein, in certain embodiments, is the use of a fetal support tissue product disclosed herein for repairing, reconstructing, replacing, or supplementing a recipient's damaged, compromised, or missing ocular tissue. In some embodiments, the fetal support tissue product is administered by parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the fetal support tissue product is administered epidurally, intrathecally, through inhalation, intravenously, or a combination thereof.

In some embodiments, the use is a homologous use. In some embodiments, the fetal support tissue product is minimally manipulated. In some embodiments, the fetal support tissue product does not comprise another article, except for water, crystalloids, or a sterilizing, preserving, or storage agent. In some embodiments, the fetal support tissue product disclosed herein does not have a systemic effect and is not dependent upon the metabolic activity of living cells for its primary function.

In some embodiments, a fetal support tissue product disclosed herein comprises proteins, glycans, protein-glycan complexes (e.g., a complex of hyaluronic acid and a heavy chain of IαI and PTX3) and enzymes that promote tissue repair. For example, the stroma of AM contains growth factors, anti-angiogenic and anti-inflammatory proteins, as well as natural inhibitors to various proteases. In some embodiments, proteins and enzymes found in a fetal support tissue product disclosed herein diffuse out of the fetal support tissue product and into the surrounding tissue.

### Treatment of Glaucoma

As used herein, “Glaucoma” means a disorder characterized by the loss of retinal ganglion cells in the optic nerve. In certain instances, glaucoma partially or fully results from an increase in intraocular pressure in the anterior chamber (AC). Intraocular pressure varies depending on the production of liquid aqueous humor by the ciliary processes of the eye and the drainage of the aqueous humor through the trabecular meshwork.

Glaucoma Drainage Devices (GDD) are medical devices that are implanted into an eye to relieve intraocular pressure by providing an alternative pathway for the aqueous humor to drain. If left uncovered, a GDD tube will erode and leave the eye susceptible to intraocular infection. Thus, the GDD tube needs to be covered. Currently, patches used to cover GDD tubes are made from pericardium, sclera and cornea. These patches are about 400-550 microns thick. The thinness of these patches results in their melting by 25% in 2 years potentially leaving the shunt tube exposed again.

In some embodiments, a fetal support tissue product disclosed herein is applied to a substrate and the substrate/fetal support tissue product is used to cover GDD tubes. In some embodiments, the substrate/fetal support tissue product is 300-600 microns thick. In some embodiments, the substrate/fetal support tissue product does not melt by 25% in 2 years. In some embodiments, the fetal support tissue product is administered by parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the fetal support tissue product is administered epidurally, intrathecally, through inhalation, intravenously, or a combination thereof.

### Treatment of Ocular Ulcers

In some embodiments, a fetal support tissue product disclosed herein is applied to a substrate and the substrate/fetal support tissue product is used to cover persistent epithelial defects and/or ulcers in eyes. In some embodiments, the fetal support tissue product is administered by parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the fetal support tissue product is administered epidurally, intrathecally, through inhalation, intravenously, or a combination thereof.

In some embodiments, the base of the ulcer is debrided with surgical sponges and the poorly adherent epithelium adjacent to the edge of the ulcer is removed (e.g., to the section of the eye where the epithelium becomes quite adherent). In some embodiments, a fetal support tissue product disclosed herein is applied to a substrate and the substrate/fetal support tissue product is transferred to the recipient eye. In some embodiments, a fetal support tissue product disclosed herein is applied to a substrate and the substrate/fetal support tissue product is then secured to the eye by sutures (e.g., interrupted 10-0 nylon sutures or running 10-0 nylon sutures) with the suture knots being buried. In some embodiments, a fetal support tissue product disclosed herein is applied to a substrate and the substrate/fetal support tissue product is secured to the eye by use of fibrin glue. In some embodiments, a protective layer is applied over the fetal support tissue product/substrate or the entire eye (e.g., a contact lens). In some embodiments, the substrate/fetal support tissue product further comprises an antibiotic (e.g., neomycin, polymyxin b sulfate and dexamethasone).

### Conjunctival, Scleral, Lid, and Orbital Rim Surface Reconstruction

In some embodiments, a fetal support tissue product disclosed herein is applied to a substrate and the substrate/fetal support tissue product is used in conjunctival, scleral, lid, and orbital rim surface reconstruction. In some embodiments, damage to the conjunctival surface results from symblepharon lysis; surgical removal of tumor, lesion, and/or scar tissue; excimer laser photorefractive keratectomy and therapeutic keratectomy; or combinations thereof.

### Coronary Uses

Disclosed herein, in certain embodiments, is the use of a fetal support tissue product disclosed herein for repairing, reconstructing, replacing, or supplementing a recipient's damaged, compromised, or missing coronary tissue. In some embodiments, the fetal support tissue product is administered by parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the fetal support tissue product is administered epidurally, intrathecally, through inhalation, intravenously, or a combination thereof.

In some embodiments, the use is a homologous use. In some embodiments, the fetal support tissue product is minimally manipulated. In some embodiments, the AM does not comprise another article, except for water, crystalloids, or a sterilizing, preserving, or storage agent. In some embodiments, the fetal support tissue product does not have a systemic effect and is not dependent upon the metabolic activity of living cells for its primary function.

In some embodiments, a fetal support tissue product disclosed herein comprises proteins, glycans, protein-glycan complexes (e.g., a complex of hyaluronic acid and a heavy chain of IαI and PTX3) and enzymes that promote tissue repair. For example, the stroma of AM contains growth factors, anti-angiogenic and anti-inflammatory proteins, as well as natural inhibitors to various proteases. In some embodiments, proteins and enzymes found in the fetal support tissue product diffuse out of the fetal support tissue product and into the surrounding tissue.

### Coronary Artery Bypass

Disclosed herein, is the use of a fetal support tissue product described herein in coronary artery bypass surgery. In some embodiments, a fetal support tissue product disclosed herein is applied to a substrate and the substrate/fetal support tissue product is grafted onto a coronary artery to bypass a section of the artery that is characterized by atherosclerosis. In some embodiments, the fetal support tissue product is administered by parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the fetal support tissue product is administered epidurally, intrathecally, through inhalation, intravenously, or a combination thereof.

### Heart Valves

In some embodiments, a fetal support tissue product disclosed herein is applied to a substrate and the substrate/fetal support tissue product is applied over a heart valve. In some embodiments, a fetal support tissue product disclosed herein is applied to a substrate and the substrate/fetal support tissue product is used as structural (tectonic) support for a heart valve. In some embodiments, a fetal support tissue product disclosed herein is applied to a substrate and the substrate/fetal support tissue product is used as a replacement for a heart valve. In some embodiments, the fetal support tissue product is administered by parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the fetal support tissue product is administered epidurally, intrathecally, through inhalation, intravenously, or a combination thereof.

### Veins and Arteries

In some embodiments, a fetal support tissue product disclosed herein is applied to a substrate and the substrate/fetal support tissue product is applied to a vein or artery. In some embodiments, a fetal support tissue product disclosed herein is applied to a substrate and the substrate/fetal support tissue product is used as structural (tectonic) support for a vein or artery. In some embodiments, the fetal support tissue product is administered by parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the fetal support tissue product is administered epidurally, intrathecally, through inhalation, intravenously, or a combination thereof

### Nerve Uses

Disclosed herein, in certain embodiments, is the use of a fetal support tissue product disclosed herein for repairing, reconstructing, replacing, or supplementing a recipient's damaged, compromised, or missing nerve tissue. In some embodiments, the fetal support tissue product is administered by parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the fetal support tissue product is administered epidurally, intrathecally, through inhalation, intravenously, or a combination thereof.

In some embodiments, the use is a homologous use. In some embodiments, the fetal support tissue product is minimally manipulated. In some embodiments, the fetal support tissue product does not comprise another article, except for water, crystalloids, or a sterilizing, preserving, or storage agent. In some embodiments, the fetal support tissue product does not have a systemic effect and is not dependent upon the metabolic activity of living cells for its primary function.

In some embodiments, a fetal support tissue product disclosed herein comprises proteins, glycans, protein-glycan complexes (e.g., a complex of hyaluronic acid and a heavy chain of IαI and PTX3) and enzymes that promote tissue repair. For example, the stroma of AM contains growth factors, anti-angiogenic and anti-inflammatory proteins, as well as natural inhibitors to various proteases. In some embodiments, proteins and enzymes found in a fetal support tissue product disclosed herein diffuse out of the fetal support tissue product and into the surrounding tissue.

In some embodiments, a fetal support tissue product disclosed herein is applied to a substrate and the substrate/fetal support tissue product is used as a covering over a nerve (e.g., a peripheral nerve). In some embodiments, a fetal support tissue product disclosed herein is applied to a substrate and the substrate/fetal support tissue product is used as a covering over a nerve graft, nerve transfer, or a repaired nerve. In some embodiments, a fetal support tissue product disclosed herein is applied to a substrate and the substrate/fetal support tissue product is used as a covering over an incision in a nerve (e.g., a peripheral nerve). In some embodiments, a fetal support tissue product disclosed herein is applied to a substrate and the substrate/fetal support tissue product is used as structural (tectonic) support for a nerve (e.g., a peripheral nerve). In some embodiments, a fetal support tissue product disclosed herein prevents adhesion in nerve repair.

In some embodiments, a fetal support tissue product disclosed herein is applied to a substrate and the substrate/fetal support tissue product is used as a non-constricting encasement for injured nerves. In some embodiments, a fetal support tissue product described herein prevents or minimizes scar formation, encapsulation, chronic compression, tethering of a nerve, and nerve entrapment. In some embodiments, a fetal support tissue product described herein prevents or minimizes neuroma formation. In some embodiments, a fetal support tissue product described herein prevents or minimizes the migration of endogenous growth factors (i.e. Nerve Growth Factor) present during nerve repair.

### Spinal Uses

Disclosed herein, in certain embodiments, is the use of a fetal support tissue product described herein during spinal surgery.

In some embodiments, a fetal support tissue product described herein is used during a laminectomy. In some embodiments, the use is a homologous use. In some embodiments, the fetal support tissue product is minimally manipulated. In some embodiments, the fetal support tissue product does not comprise another article, except for water, crystalloids, or a sterilizing, preserving, or storage agent. In some embodiments, the fetal support tissue product does not have a systemic effect and is not dependent upon the metabolic activity of living cells for its primary function.

In some embodiments, a fetal support tissue product disclosed herein comprises proteins, glycans, protein-glycan complexes (e.g., a complex of hyaluronic acid and a heavy chain of IαI and PTX3) and enzymes that promote tissue repair. For example, the stroma of AM contains growth factors, anti-angiogenic and anti-inflammatory proteins, as well as natural inhibitors to various proteases. In some embodiments, proteins and enzymes found in a fetal support tissue product disclosed herein diffuse out of the fetal support tissue product and into the surrounding tissue. In some embodiments, the fetal support tissue product is administered by parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the fetal support tissue product is administered epidurally, intrathecally, through inhalation, intravenously, or a combination thereof.

In some embodiments, a fetal support tissue product described herein is used to reduce or prevent epidural fibrosis and/or scar adhesions following spinal surgery (e.g., laminectomy). In some embodiments, a fetal support tissue product described herein is implanted between dura mater and overlying tissue following spinal surgery (e.g., laminectomy). In some embodiments, implanting a fetal support tissue product described herein between dura mater and overlying tissue following spinal surgery (e.g., laminectomy) reduces or prevents migration of fibroblasts to the dura mater and collagen deposition on the dura mater.

In some embodiments, a fetal support tissue product described herein is used to reduce or prevent the development of proliferative scarring following spinal surgery (e.g., laminectomy). In some embodiments, a fetal support tissue product described herein is used to reduce or prevent the development of a postoperative (e.g., postlaminectomy) epidural/peridural/perineural scar. In some embodiments, a fetal support tissue product described herein is used to reduce or prevent the development of proliferative scarring following spinal surgery (e.g., laminectomy). In some embodiments, a fetal support tissue product disclosed herein is used to reduce or prevent the development of a postlaminectomy membrane.

In some embodiments, a fetal support tissue product described herein is used to reduce or prevent the development of extradural compression or dural tethering following spinal surgery (e.g., laminectomy). In some embodiments, a fetal support tissue product described herein is used to reduce or prevent the development of tethered nerve roots following spinal surgery (e.g., laminectomy). In some embodiments, a fetal support tissue product described herein is used to reduce or prevent the development of arachnoiditis following spinal surgery (e.g., laminectomy).

In some embodiments, a fetal support tissue product disclosed herein further comprises morselized bone tissue. In some embodiments, a fetal support tissue product disclosed herein comprising morselized bone tissue is used during a spinal fusion procedure. In some embodiments, a fetal support tissue product disclosed herein comprising morselized bone tissue is implanted between adjacent vertebrae. In some embodiments, implantation of a fetal support tissue product disclosed herein comprising morselized bone tissue between two adjacent vertebrae promotes fusion of the vertebrae.

In some embodiments, a fetal support tissue product disclosed herein is used as a protective graft over an incision in the dura mater. In some embodiments, a fetal support tissue product disclosed herein is applied to a substrate and the substrate/fetal support tissue product is used as structural (tectonic) support for the dura mater. In some embodiments, a fetal support tissue product disclosed herein is applied to a substrate and the substrate/fetal support tissue product is used as a replacement for the dura mater.

### Miscellaneous Uses of a Fetal Support Tissue Product

In some embodiments, a fetal support tissue product disclosed herein is applied to a patch or wound dressing. In some embodiments, the fetal support tissue product is administered by parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural, and/or subcutaneous). In some embodiments, the fetal support tissue product is administered epidurally, intrathecally, through inhalation, intravenously, or a combination thereof.

In some embodiments, a fetal support tissue product disclosed herein is used as a dermal filler. In some embodiments, a fetal support tissue product disclosed herein is injected into subdermal facial tissues. In some embodiments, a fetal support tissue product disclosed herein is injected under wrinkles and aging lines of the face (e.g., nasolabial folds, melomental folds, “crow's feet” and forehead wrinkles). In some embodiments, a fetal support tissue product disclosed herein is used for lip augmentation. In some embodiments, a fetal support tissue product disclosed herein is injected into the lips.

In some embodiments, a fetal support tissue product disclosed herein is used to treat arthritis (e.g., osteoarthritis, rheumatoid arthritis, septic arthritis, ankylosing spondylitis, spondylosis). In some embodiments, a fetal support tissue product disclosed herein is injected into an arthritic joint (e.g., a knee).

In some embodiments, a fetal support tissue product disclosed herein is used to inhibit bone resorption in an individual in need thereof. In some embodiments, the individual has arthritis, osteoporosis, alveolar bone degradation, Paget's disease, or a bone tumor. In some embodiments, the fetal support tissue product is injected into a joint. In some embodiments, the fetal support tissue product is contacted with a bone (e.g., by use of a wound dressing or bandage). In some embodiments, the fetal support tissue product coats a bone stent, bone implant, or bone prosthesis (e.g., an osseointegrated implant). As used herein, an “osseointegrated implant” means a three dimensional implant containing pores into which osteoblasts and supporting connective tissue can migrate. In some embodiments, the bone stents are inserted into the intramedullary canal of a bone. In some embodiments, the bone stent is placed in the sinus tarsi. In some embodiments, the bone stent in placed in a knee or joint. In some embodiments, the bone stent is placed in a bone fracture. In some embodiments, the bone stent is expandable or contractible.

In some embodiments, a fetal support tissue product disclosed herein is used to promote or induce bone formation in an individual in need thereof in an individual in need thereof. In some embodiments, the individual has arthritis, osteoporosis, alveolar bone degradation, Paget's disease, or a bone tumor. In some embodiments, the fetal support tissue product is injected into a joint. In some embodiments, the fetal support tissue product is contacted with a bone (e.g., by use of a wound dressing or bandage). In some embodiments, the fetal support tissue product coats a bone stent, bone implant, or bone prosthesis (e.g., an osseointegrated implant). As used herein, an “osseointegrated implant” means a three dimensional implant containing pores into which osteoblasts and supporting connective tissue can migrate. In some embodiments, the bone stents are inserted into the intramedullary canal of a bone. In some embodiments, the bone stent is placed in the sinus tarsi. In some embodiments, the bone stent in placed in a knee or joint. In some embodiments, the bone stent is placed in a bone fracture. In some embodiments, the bone stent is expandable or contractible.

In some embodiments, a fetal support tissue product disclosed herein is used to inhibit osteoclast differentiation. In some embodiments, the individual has arthritis, osteoporosis, alveolar bone degradation, Paget's disease, or a bone tumor. In some embodiments, the fetal support tissue product is injected into a joint. In some embodiments, the fetal support tissue product is contacted with a bone (e.g., by use of a wound dressing or bandage). In some embodiments, the fetal support tissue product coats a bone stent, bone implant, or bone prosthesis (e.g., an osseointegrated implant). As used herein, an “osseointegrated implant” means a three dimensional implant containing pores into which osteoblasts and supporting connective tissue can migrate. In some embodiments, the bone stents are inserted into the intramedullary canal of a bone. In some embodiments, the bone stent is placed in the sinus tarsi. In some embodiments, the bone stent in placed in a knee or joint. In some embodiments, the bone stent is placed in a bone fracture. In some embodiments, the bone stent is expandable or contractible.

In some embodiments, a fetal support tissue product disclosed herein is used to promote mineralization by osteoblasts in an individual in need thereof. In some embodiments, the individual has arthritis, osteoporosis, alveolar bone degradation, Paget's disease, or a bone tumor. In some embodiments, the fetal support tissue product is injected into a joint. In some embodiments, the fetal support tissue product is contacted with a bone (e.g., by use of a wound dressing or bandage). In some embodiments, the fetal support tissue product coats a bone stent, bone implant, or bone prosthesis (e.g., an osseointegrated implant). As used herein, an “osseointegrated implant” means a three dimensional implant containing pores into which osteoblasts and supporting connective tissue can migrate. In some embodiments, the bone stents are inserted into the intramedullary canal of a bone. In some embodiments, the bone stent is placed in the sinus tarsi. In some embodiments, the bone stent in placed in a knee or joint. In some embodiments, the bone stent is placed in a bone fracture. In some embodiments, the bone stent is expandable or contractible.

In some embodiments, a fetal support tissue product disclosed herein is used to balance bone resorption and bone formation in an individual in need thereof. In some embodiments, the individual has arthritis, osteoporosis, alveolar bone degradation, Paget's disease, or a bone tumor. In some embodiments, the fetal support tissue product is injected into a joint. In some embodiments, the fetal support tissue product is contacted with a bone (e.g., by use of a wound dressing or bandage). In some embodiments, the fetal support tissue product coats a bone stent, bone implant, or bone prosthesis (e.g., an osseointegrated implant). As used herein, an “osseointegrated implant” means a three dimensional implant containing pores into which osteoblasts and supporting connective tissue can migrate. In some embodiments, the bone stents are inserted into the intramedullary canal of a bone. In some embodiments, the bone stent is placed in the sinus tarsi. In some embodiments, the bone stent in placed in a knee or joint. In some embodiments, the bone stent is placed in a bone fracture. In some embodiments, the bone stent is expandable or contractible.

In some embodiments, a fetal support tissue product disclosed herein is used to treat an orthodontic or a periodontal condition. In some embodiments, the periodontal condition is selected from gingivitis, gingival recession or periodontitis. In some embodiments, a fetal support tissue product disclosed herein is used as an anti-inflammatory or used to promote osteointegration or healing. In some embodiments, a fetal support tissue product disclosed herein is used in combination with a dental implant to promote implant osteointegration, anti-inflammation, and healing.

In some embodiments, a fetal support tissue product disclosed herein to treat hoarseness or voice disorders. In some embodiments, a fetal support tissue product disclosed herein is used for injection laryngoplasty to repair vocal cords.

In some embodiments, a fetal support tissue product disclosed herein is coated onto a medical implant (e.g., a stent). In some embodiments, a medical implant/fetal support tissue product disclosed herein is implanted into an individual in need thereof, wherein the fetal support tissue product is partially or fully released into the individual. In some embodiments, the medical implant is a stent (e.g., a bone stent or a coronary stent). In some embodiments, the medical implant is a bone stent. In some embodiments, the medical implant is a coronary stent.

### Combination Treatments

In some embodiments, the compositions and methods described herein are used in conjunction with other well-known therapeutic reagents that are selected for their particular usefulness against the condition that is being treated. In general, the compositions described herein and, in embodiments where combinational therapy is employed, other agents do not have to be administered in the same composition, and may, because of different physical and chemical characteristics, have to be administered by different routes. The determination of the mode of administration and the advisability of administration, where possible, in the same composition, is well within the knowledge of the skilled clinician. The initial administration can be made according to established protocols known in the art, and then, based upon the observed effects, the dosage, modes of administration and times of administration can be modified by the skilled clinician.

The particular choice of compounds used will depend upon the diagnosis of the attending physicians and their judgment of the condition of the patient and the appropriate treatment protocol. In some embodiments, the compounds are administered concurrently (e.g., simultaneously, essentially simultaneously or within the same treatment protocol) or sequentially, depending upon the nature of the disease, disorder, or condition, the condition of the patient, and the actual choice of compounds used. The determination of the order of administration, and the number of repetitions of administration of each therapeutic agent during a treatment protocol, is well within the knowledge of the skilled physician after evaluation of the disease being treated and the condition of the patient.

It is known to those of skill in the art that therapeutically-effective dosages can vary when the drugs are used in treatment combinations. Methods for experimentally determining therapeutically-effective dosages of drugs and other agents for use in combination treatment regimens are described in the literature. For example, the use of metronomic dosing, i.e., providing more frequent, lower doses in order to minimize toxic side effects, has been described extensively in the literature. Combination treatment further includes periodic treatments that start and stop at various times to assist with the clinical management of the patient.

For combination therapies described herein, dosages of the co-administered compounds will of course vary depending on the type of co-drug employed, on the specific drug employed, on the disease or condition being treated and so forth. In addition, in some embodiments, when co-administered with one or more biologically active agents, the compound provided herein is administered either simultaneously with the biologically active agent(s), or sequentially. If administered sequentially, the attending physician will decide on the appropriate sequence of administering protein in combination with the biologically active agent(s).

In some embodiments, multiple therapeutic agents are administered in any order, or even simultaneously. If simultaneously, in some embodiments, the multiple therapeutic agents are provided in a single, unified form, or in multiple forms (by way of example only, either as a single pill or as two separate pills). In some embodiments, one of the therapeutic agents is given in multiple doses, or both are given as multiple doses. If not simultaneous, in some embodiments, the timing between the multiple doses varies from more than zero weeks to less than four weeks. In addition, the combination methods, compositions and formulations are not to be limited to the use of only two agents; the use of multiple therapeutic combinations are also envisioned.

It is understood that the dosage regimen to treat or ameliorate the condition(s) for which relief is sought, can be modified in accordance with a variety of factors. These factors include the disorder from which the subject suffers, as well as the age, weight, sex, diet, and medical condition of the subject. Thus, the dosage regimen actually employed can vary widely and therefore can deviate from the dosage regimens set forth herein.

### Kits/Articles of Manufacture

For use in the therapeutic applications described herein, kits and articles of manufacture are also described herein. Such kits can include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers can be formed from a variety of materials such as glass or plastic.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, bottles, and any packaging material suitable for a selected formulation and intended mode of administration and treatment. A wide array of formulations of the compounds and compositions provided herein are contemplated as are a variety of treatments for any disease, disorder, or condition.

For example, the container(s) can include one or more UCAM compositions described herein, optionally in a composition or in combination with another agent as disclosed herein. The container(s) optionally have a sterile access port (for example the container can be an intravenous solution bag or a vial having a stopper pierceable by a hypodermic injection needle). Such kits optionally comprising a compound with an identifying description or label or instructions relating to its use in the methods described herein.

A kit will typically include one or more additional containers, each with one or more of various materials (such as reagents, optionally in concentrated form, and/or devices) desirable from a commercial and user standpoint for use of the compositions described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

In certain embodiments, the compositions can be presented in a pack or dispenser device which can contain one or more unit dosage forms containing a compound provided herein. The pack can for example contain metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions containing a compound provided herein formulated in a compatible 1 carrier can also be prepared, placed in an appropriate container, and labeled for treatment of an indicated condition.

### Certain Terminology

As used herein, “fetal support tissue” means tissue used to support the development of a fetus. Examples of fetal support tissue include, but are not limited to, placental amniotic membrane (PAM), or substantially isolated PAM, (umbilical cord amniotic membrane (UCAM) or substantially isolated UCAM, chorion or substantially isolated chorion, amnion-chorion or substantially isolated amnion-chorion, placenta or substantially isolated placenta, umbilical cord or substantially isolated umbilical cord, or any combinations thereof.

As used herein, “fetal support tissue product” means any product resulting from grinding fetal support tissue. Examples of fetal support tissue include, but are not limited to, placental amniotic membrane (PAM), or substantially isolated PAM, umbilical cord amniotic membrane (UCAM) or substantially isolated UCAM, chorion or substantially isolated chorion, amnion-chorion or substantially isolated amnion-chorion, placenta or substantially isolated placenta, umbilical cord or substantially isolated umbilical cord, or any combinations thereof.

As used herein, “powder” means matter in the form of fine dry particles. In some embodiments, the particles are not uniform in size. In some embodiments, the particles are substantially uniform in size.

As used herein, “grinding” means any method of reducing fetal support tissue to small particle or a powder. The term grinding includes pulverizing, homogenizing, filing, milling, grating, pounding, and crushing.

As used herein, “placenta” means the organ that connects a developing fetus to the maternal uterine wall to allow nutrient uptake, waste elimination, and gas exchange via the maternal blood supply. The placenta is composed of three layers. The innermost placental layer surrounding the fetus is called amnion. The allantois is the middle layer of the placenta (derived from the embryonic hindgut); blood vessels originating from the umbilicus traverse this membrane. The outermost layer of the placenta, the chorion, comes into contact with the endometrium. The chorion and allantois fuse to form the chorioallantoic membrane.

As used herein, “chorion” means the membrane formed by extraembryonic mesoderm and the two layers of trophoblast. The chorionic villi emerge from the chorion, invade the endometrium, and allow transfer of nutrients from maternal blood to fetal blood. The chorion consists of two layers: an outer formed by the trophoblast, and an inner formed by the somatic mesoderm; the amnion is in contact with the latter. The trophoblast is made up of an internal layer of cubical or prismatic cells, the cytotrophoblast or layer of Langhans, and an external layer of richly nucleated protoplasm devoid of cell boundaries, the syncytiotrophoblast. The avascular amnion is adherent to the inner layer of the chorion.

As used herein, “amnion-chorion” means a product comprising amnion and chorion. In some embodiments, the amnion and the chorion are not separated (i.e., the amnion is naturally adherent to the inner layer of the chorion). In some embodiments, the amnion is initially separated from the chorion and later combined with the chorion during processing.

As used herein, “umbilical cord” means the organ that connects a developing fetus to the placenta. The umbilical cord is composed of Wharton's jelly, a gelatinous substance made largely from mucopolysaccharides. It contains one vein, which carries oxygenated, nutrient-rich blood to the fetus, and two arteries that carry deoxygenated, nutrient-depleted blood away.

As used herein, “placental amniotic membrane” (PAM) means amniotic membrane derived from the placenta. In some embodiments, the PAM is substantially isolated.

As used herein, “umbilical cord amniotic membrane” (UCAM) means amniotic membrane derived from the umbilical cord. UCAM is a translucent membrane. The UCAM has multiple layers an epithelial layer, a basement membrane; a compact layer; a fibroblast layer; and a spongy layer. It lacks blood vessels or a direct blood supply. In some embodiments, the UCAM is substantially isolated. In some embodiments, the UCAM comprises Wharton's Jelly. In some embodiments, the UCAM comprises blood vessels and/or arteries. In some embodiments, the UCAM comprises Wharton's Jelly and blood vessels and/or arteries.

“Substantially isolated” or “isolated” means that the fetal support tissue product has been separate from undesired materials (e.g., red blood cells, blood vessels, and arteries) derived from the original source organism. Purity, or “isolation” may be assayed by standard methods, and will ordinarily be at least about 10% pure, more ordinarily at least about 20% pure, generally at least about 30% pure, and more generally at least about 40% pure; in further embodiments at least about 50% pure, or more often at least about 60% pure; in still other embodiments, at least about 95% pure.

As used herein, “biological activity” means the activity of polypeptides and polysaccharides. In some embodiments, the activity of polypeptides and polysaccharides found in umbilical cord (and substantially isolated umbilical cord), UCAM (and substantially isolated UCAM), placenta (and substantially isolated placenta), PAM (and substantially isolated PAM), chorion (and substantially isolated chorion), or amnion-chorion (and substantially isolated amnion-chorion).

As used herein, the substantial preservation of biological activity or structural integrity means that when compared to the biological activity and structural integrity of non-processed tissue, the biological activity and structural integrity of the fetal support tissue product has only decreased by about 5%, about 10%, about 15%, about 20%, about 25%, about 30%, about 35%, about 40%, about 50%, about 60%.

The term “fresh” refers to tissue that is less than 10 days old following birth, and which is in substantially the same form as it was following birth.

The terms “subject” and “individual” are used interchangeably. As used herein, both terms mean any animal, preferably a mammal, including a human or non-human. The terms patient, subject, and individual are used interchangeably. None of the terms are to be interpreted as requiring the supervision of a medical professional (e.g., a doctor, nurse, physician's assistant, orderly, hospice worker).

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### EXAMPLES

### Example 1. Processing Method

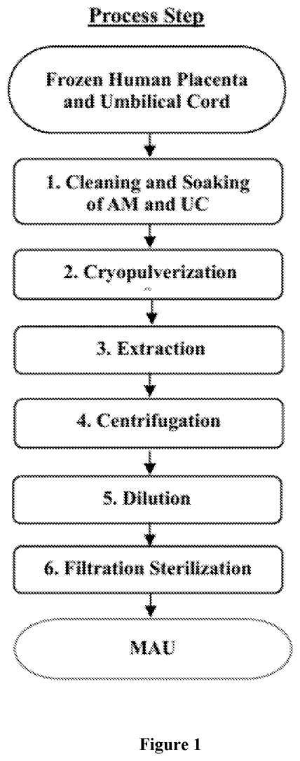

FIG. 1 shows a flow chart, illustrating an example of the methods of processing fetal support tissue disclosed herein. Fetal support tissues—Amniotic membrane and umbilical cord—are harvested from a donor and cleaned. The cleaned amniotic membrane and umbilical cord are cryopulverized with a FreezerMill for extraction. The cryopulverized amniotic membrane and umbilical cord are extracted in water for injection. The extract is centrifuged to remove large tissue particles. The amniotic membrane and umbilical cord are then diluted with water for injection with a dilution factor of 2 by mixing at 20 rpm at 4° C. for 30 min. The diluted extract in saline is then filtered with a 0.45 μm filter followed by a 0.2 μm filter.

### Example 2. Extraction Time Effect on Protein Recovery

Cryopulverized amniotic membrane (“AM”) and umbilical cord (“UC”) tissues were sequentially extracted in saline for 1 hour (h), 2 h or 3 h, respectively, and the respective pellet was extracted with 6 M GnHCl/PBS for 24 hours.

AM and UC from a donor were processed and cryopulverized. The cryopulverized AM/UC was extracted in saline at 1:4 (w/v, g/ml) ratio using a tube rotator at 20 rpm at 4° C. for 1, 2 or 3 h (n=2) to produce a MAU/saline extract. The MAU/saline extract was collected from the supernatant after centrifugation at 48,000 g at 4° C. for 30 min. Remaining pellets were washed for 2 times with saline (10 ml saline/1 g pellet) and extracted with 6 M GnHCl/PBS with PI (10 mM EDTA, 2 mM PMSF) at 1:4 (w/v, g/ml) ratio at 20 rpm at 4° C. for 24 h. The GnHCl extract was collected after centrifugation at 48,000 g at 4° C. for 30 min. Both MAU/saline extract and 6 M GnHCl extract were tested with HA assay for HA content, BCA assay for total proteins, and Western blot analysis for HC-HA/PTX3 content. The efficiency in the above extraction was determined by what can be obtained in MAU/saline extract and/or what is left in the 6 M GnHCl extract.

Table 1 summarizes the overall recovery rate of HA and total proteins. The results showed that the recovery rate achieved 82.8±0.7% of total HA after 1 h and did not increase with further increase of the extraction time to 2 h or 3 h (all p>0.05 vs 1 h). Extraction in saline for 1 h also achieved the recovery rate of 9.5±0.1% of total proteins, which did not increase with further increase of the extraction time (all p>0.05 vs 1 h). Therefore, the extraction time in saline is optimized at 1 h.

TABLE 1
Total HA and proteins in extracts with different extraction time

| | | MAU/Saline (n = 2) | | MAU/GnHCl (n = 2) | |
|---|---|---|---|---|---|
| | | HA | Total | HA | Total |
| HA | | (μg/g wet) | % | (μg/g wet) | % |
| 1 | Saline 1 h | 1051 ± 123 | 82.8 ± 0.7 | 217 ± 14 | 17.2 ± 0.7 |
| 2 | Saline 2 h | 1022 ± 66 | 83.1 ± 1.9 | 207 ± 15 | 16.9 ± 1.9 |
| 3 | Saline 3 h | 1105 ± 250 | 82.9 ± 2.5 | 224 ± 12 | 17.1 ± 2.5 |
| | | Protein | Total | Protein | Total |
| Protein | | (μg/g wet) | % | (μg/g wet) | % |
| 1 | Saline 1 h | 137 ± 2 | 9.5 ± 0.1 | 1305 ± 7 | 90.5 ± 0.1 |
| 2 | Saline 2 h | 128 ± 40 | 8.5 ± 3.1 | 1389 ± 117 | 91.5 ± 3.1 |
| 3 | Saline 3 h | 130 ± 1 | 9.1 ± 0.9 | 1303 ± 132 | 90.9 ± 0.9 |

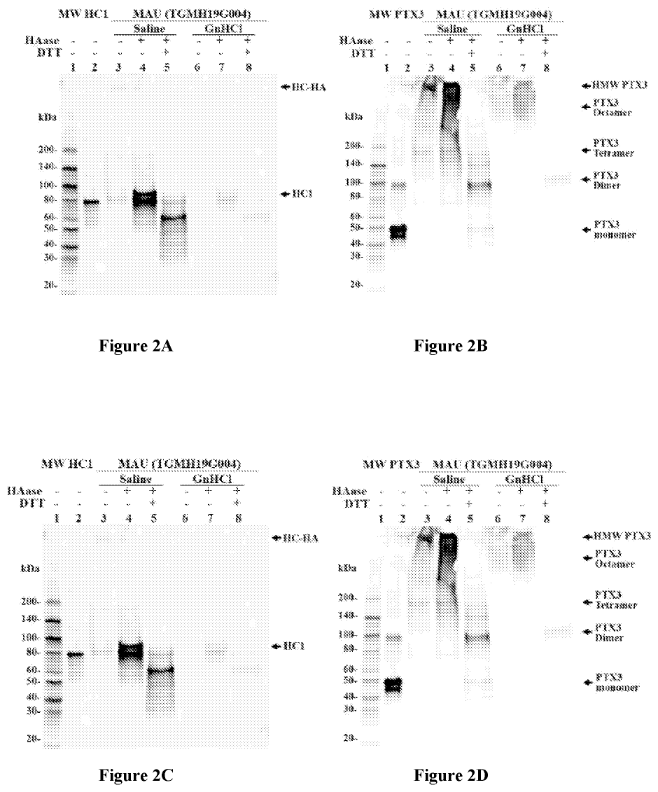

The result showed that saline extract showed strong 80 kDa HC1 and HMW PTX3 smear released from the BMW HC-HA/PTX3 retained in the loading well after digestion of HA by HAase, while GnHCl extract only showed weak HC1 and PTX3 smear after HAase digestion without HMW HC-HA/PTX3 in the loading well (FIGS. 2A-2B). This result strongly suggested that the majority of HC-HA/PTX3 was extracted by saline after 1 h.

### Example 3. Centrifuge Time Effect on Protein Recovery

Cryopulverized AM/UC tissue was extracted in saline for 1 h and centrifuged at different speeds (i.e. 14,000 rcf, 10,000 rcf, 3200rcf, and 48,000 rcf (as the control)).

The results are shown in Table 2. Centrifugation at 3200 rcf yielded visible particles suspended in the solution and settled after 30 min. Similar visible particles were also noticed but in a smaller amount with centrifugation at 10,000 rcf but not at 14,000 rcf and 48,000 rcf. The pellet was washed for 2 times in saline and then extracted with 6 M GnHCl/PBS at 1:4 (w/v, g/ml) at 4° C. for 24 h; the supernatant was collected after centrifugation at 48,000 g at 4° C. for 30 min for analysis. Both supernatants were tested for Western blot analysis. The efficiency in the above centrifugation was determined by what can be obtained in the saline extract and/or what is left in the 6 M GnHCl extract.

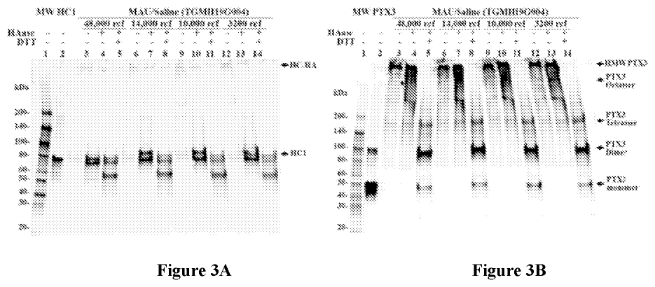

The results suggest that centrifugation at 14,000 rcf or more can be used to prepare supernatant for further preparation of the umbilical cord/amniotic membrane fetal support tissue product (“MAU”). This notion was further supported by biochemical quantification of HA and protein summarized in Table 2 and Western blot analysis for HC-HA/PTX3 (FIGS. 3A-3B), which showed that centrifugation at different speeds does not affect the content of HA, protein and HC-HA/PTX3 in the extract.

TABLE 2
Total HA and proteins in extracts with different centrifuge speed

| | MAU/Saline (n = 2) | | | MAU/GnHCI (n = 2) | | |
|---|---|---|---|---|---|---|
| | HA | Total | p value vs | HA | Total | p value vs |
| HA | (μg/g wet) | % | Control | (μg/g wet) | % | Control |
| Saline | 1280 ± 116 | 88 ± 0.4 | N/A | 167 ± 50 | 12 ± 0.04 | N/A |
| 48,000 rcf | | | | | | |
| (Control) | | | | | | |
| Saline | 1296 ± 30 | 88 ± 0.002 | 0.882 | 180 ± 8 | 12 ± 0.002 | 0.773 |
| 14,000 rcf | | | | | | |
| Saline | 1261 ± 55 | 87 ± 0.001 | 0.856 | 182 ± 8 | 13 ± 0.001 | 0.744 |
| 10,000 rcf | | | | | | |
| Saline | 1293 ± 155 | 90 ± 0.02 | 0.93 | 147 ± 7 | 10 ± 0.02 | 0.677 |
| 3,200 rcf | | | | | | |
| | Protein | Total | p value vs | Protein | Total | p value vs |
| Protein | (μg/g wet) | % | Control | (μg/g wet) | % | Control |
| Saline | 279 ± 9 | 15 ± 0.1 | N/A | 1598 ± 183 | 85 ± 0.02 | N/A |
| 48,000 rcf | | | | | | |
| (Control) | | | | | | |
| Saline | 322 ± 18 | 16 ± 0.003 | 0.134 | 1657 ± 125 | 84 ± 0.002 | 0.746 |
| 14,000 rcf | | | | | | |
| Saline | 318 ± 15 | 16 ± 0.01 | 0.109 | 1706 ± 54 | 84 ± 0.01 | 0.555 |
| 10,000 rcf | | | | | | |
| Saline | 374 ± 18 | 20 ± 0.003 | 0.045 | 1530 ± 105 | 80 ± 0.003 | 0.702 |
| 3,200 rcf | | | | | | |

### Example 4. Excipient Effect on Protein Recovery

Cryopulverized MAU was prepared and extracted in three different excipients. Table 3 summarizes the data of biochemical quantification of HA and total proteins for MAU prepared in three different excipients, i.e., MAU/saline, MAU/WFI, and MAU/SW. WFI extracted comparable HA to that extracted by saline (p>0.05) with undetectable protein. HA and protein left in the pellet could still be extracted by GnHCl. In contrast, SW extracted less HA compared to saline and WFI (both p<0.05) with undetectable protein. These results suggest that SW is not acceptable as an excipient for MAU preparation.

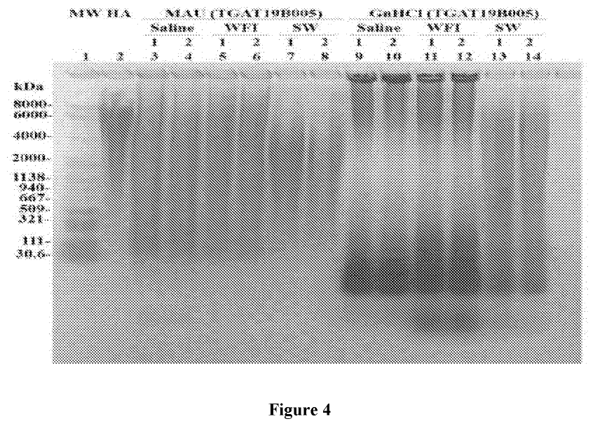

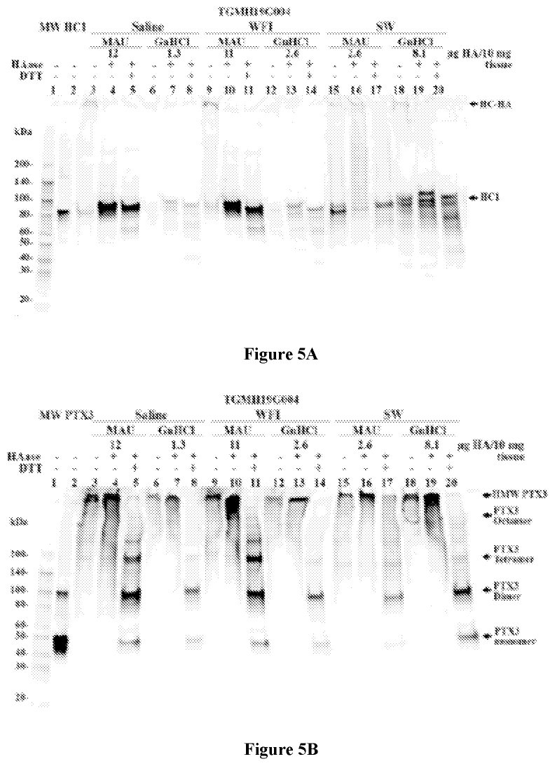

Agarose gel analysis for HA size distribution confirmed that WFI extracted similar amounts and size of HA as saline, but SW not only extracted less HA but also extracted lower MW HA compared to saline and WFI (FIG. 4). Further analysis with Western blot, which was used to semi-quantitate and assess the integrity of HC-HA/PTX3, showed that WFI successfully extracted the majority of HC-HA/PTX3 as saline; however, SW only extracted a minority of HC-HA/PTX3 with some degraded (FIGS. 5A-5B). This result suggested that WFI extracts comparable HC-HA/PTX3 to saline but SW extracts less and damaged HC-HA/PTX3. Collectively, it can be concluded from the above data that both saline and WFI are acceptable excipients for MAU, but that SW is not acceptable.

TABLE 3
Total HA and proteins in sequential saline, WFI or SW and GnHCl extracts

| | MAU (n = 2) | | | GnHCI (n = 2) | | | | |
|---|---|---|---|---|---|---|---|---|
| | | | p value | | | p value | Total | p value |
| | HA | Total | vs Saline | HA | Total | vs Saline | HA | vs Saline |
| HA | (μg/g wet) | % | (vs WFI) | (μg/g wet) | % | (vs WFI) | (μg/g wet) | (vs WFI) |
| Saline | 1240 ± 2 | 90 ± 0.001 | N/A | 139 ± 6 | 10 ± 0.003 | N/A | 1379 ± 4 | N/A |
| WFI | 1139 ± 58 | 81 ± 0.01 | 0.243 | 265 ± 4 | 19 ± 0.01 | 0.003 | 1404 ± 53 | 0.627 |
| SW | 251 ± 20 | | 0.009 | 842 ± 0.2 | 77 ± 0.01 | 0.001 | 1086 ± 30 | 0.042 |
| | | | (0.016) | | | (0.002) | | (0.033) |
| | | | p value | | | p value | | p value |
| | Protein | Total | vs Saline | Protein | Total | vs Saline | Protein | vs Saline |
| Protein | (μg/g wet) | % | (vs WFI) | (μg/g wet) | % | (vs WFI) | (μg/g wet) | (vs WFI) |
| Saline | 130 ± 9 | 8 ± 0.01 | N/A | 1514 ± 68 | 92 ± 0.03 | N/A | 1645 ± 41 | N/A |
| WFI | Undetectable | 0 ± 0 | N/A | 1950 ± 21 | 100 ± 0 | 0.051 | 1950 ± 21 | 0.06 |
| SW | Undetectable | 0 ± 0 | N/A | 1938 ± 89 | 100 ± 0 | 0.038 | 1938 ± 89 | 0.075 |
| | | | | | | (0.88) | | (0.88) |

### Example 5. Terminal Sterilization Method Effect on Protein Recovery

To determine whether terminal sterilization can be performed by γ-irradiation, fetal support tissues—amniotic membrane and umbilical cord—were prepared in saline, WFI, or SW and subjected to terminal sterilization in dry ice to reduce the potential ill effect that is known to γ-irradiation. The AM and UC were blended in saline, WIF or SW at 1:4 (w/v) ratio at room temperature with the blending speed set at high for 15 sec followed by low for 15 sec for a total of six high/low speed cycles, centrifuged at 3,200 g for 30 min at 4° C., and filtered with a 200 μm mesh to collect the respective supernatant as MAU/saline, MAU/WIF, or MAU/SW.

The MAU/saline, MAU/WIF and MAU/SW were subjected to γ-irradiation at a dose of ±10% kGy with or without dry ice. The unsterilized and sterilized samples were tested for HA assay, BCA assay, agarose gel analysis, Coomassie blue analysis, and Western blot analysis.

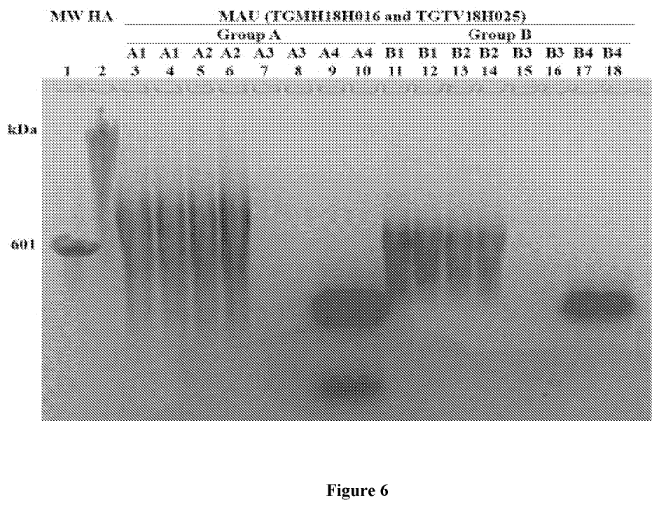

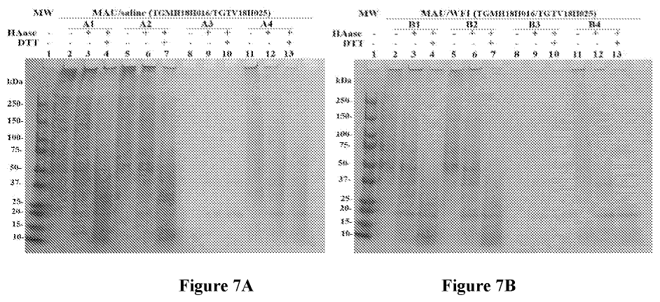

Table 4 summarizes the biochemical quantitation of HA and total proteins for MAU/saline, MAU/WFI and MAU/SW. The result showed that after γ-irradiation with or without dry ice the HA in MAU/saline and MAU/WFI dramatically decreased to undetectable or very low level (p<0.05) compared to that without γ-irradiation. The total protein in three MAUs were dramatically increased after γ-irradiation especially without dry ice. The increased protein level might be derived from protein fragments after degradation by γ-irradiation (see FIGS. 7A-7B Coomassie blue analysis). This result indicates that γ-irradiation induces damage in HA and proteins in MAUs. This finding was confirmed by agarose gel analysis for HA size distribution (FIG. 6) and Coomassie blue analysis for protein distribution (FIGS. 7A-7B), which showed complete (without dry ice) or incomplete (with dry ice) degradation of HA or protein after γ-irradiation, respectively.

TABLE 4
HA and protein content in MAU after gamma-irradiation A1, B1 and C1,
store at 4° C. A2, B2 and C2, store at −80° C.

| | | | p value | | p value |
|---|---|---|---|---|---|
| | | | vs A1, | | vs A1, |
| MAU | | HA (μg/ml) | B1 or C1 | Protein (μg/ml) | B1, C1 |
| MAU/saline | A1 | 215.0 ± 21.2 | N/A | 69.1 ± 0.6 | N/A |
| (A) | A2 | 215.3 ± 8.0 | 0.493 | 79.6 ± 8.7 | 0.115 |
| | A3 | Undetectable | N/A | 621.6 ± 94.0 | 0.007 |
| | A4 | Undetectable | N/A | 107.5 ± 1.5 | 0.0004 |
| MAU/WFI | B1 | 246.7 ± 0.07 | N/A | Undetectable | N/A |
| (B) | B2 | 261.6 ± 7.5 | 0.054 | Undetectable | |
| | B3 | Undetectable | N/A | 481.3 ± 63.0 | N/A |
| | B4 | 47.7 ± 4.0 | 0.0001 | Undetectable | N/A |
| MAU/SW | C1 | Undetectable | N/A | Undetectable | N/A |
| (C) | C2 | Undetectable | | Undetectable | |
| | C3 | Undetectable | | 97.4 ± 66.3 | N/A |
| | C4 | Undetectable | | 0.79 ± 4.0 | N/A |

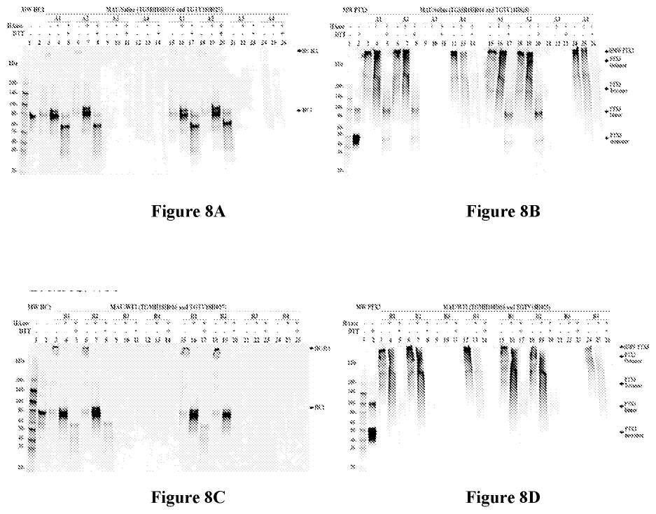

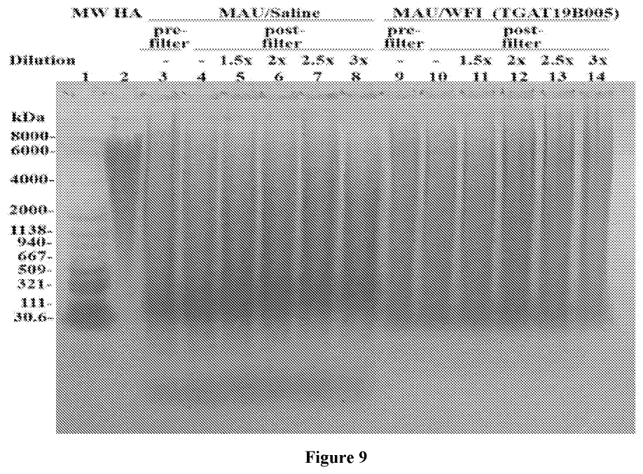

To determine whether HC-HA/PTX3 in MAUs are degraded by γ-irradiation, Western blot analysis was performed with MAU/saline and MAU/WFI. The result showed that no HC-HA/PTX3 complex was present in MAUs after γ-irradiation (FIGS. 8A-8D), suggesting that γ-irradiation also induced damage of HC-HA/PTX3 in both MAUs. In conclusion, terminal sterilization by γ-irradiation cannot be applied to MAU because of degradation of HA, proteins, and HC-HA/PTX3 in MAU/saline, MAU/WFI, and MAU/SW.

### Example 6. Filtration Sterilization and Dilution Effect on Protein Recovery

The fetal support tissue product was subject to membrane filtration sterilization with pre-filtration via a 0.45 μm filter followed by a 0.2 μm filter. To reduce the clogging of the filter and increase the recovery rate of MAU, we also compared undiluted and serially diluted MAU before filtration. The unfiltered (as the control) and all filtered MAU with or without dilution were compared by subjecting to the above assays.

AM and UC was cryopulverized, extracted in saline or WFI at 1:4 (w/v) ratio at 4° C. for 1 h, centrifuged at 48,000 g for 30 min at 4° C. and the respective supernatant was collected. MAU/saline and MAU/WIF were diluted with respective saline or WFI with a dilution factor of 1.5, 2.0 or 2.5 by mixing at 20 rpm at 4° C. for 30 min. MAU/saline and MAU/WIF with or without dilution were filtered with a 0.45 μm filter followed by a 0.2 μm filter. The unfiltered and filtered MAU/saline and MAU/WIF with or without dilution were tested for HA assay, BCA assay, agarose gel analysis, and Western blot analysis.

Table 5 summarizes the biochemical quantitation of HA and total proteins for MAU/saline and MAU/WFI after sequential filtration with 0.45 μm and 0.2 μm filters. The result showed that filtration of MAU/saline and MAU/WFI without dilution had a recovery rate of HA and total proteins at 91% and 83% (MAU/saline) or 96% and 72% (MAU/WFI), respectively, and that dilution of MAU/saline or MAU/WFI by 1.5-fold, 2-fold or 2.5-fold did not show any difference in the recovery rate of HA and total proteins compared to the undiluted one (all p>0.05). This result suggested that filtration of MAU/saline and MAU/WFI with or without dilution have no impact on the recovery rate of either HA or total proteins, and the recovery of HA and total proteins are acceptable for MAU preparation. However, as anticipated, diluted MAU is superior than undiluted ones by increasing the speed of filtration with an increase of dilution fold.

TABLE 5
Recovery of HA and proteins in MAU/saline and MAU/WFI after filtration

| | | | | | Post-filter | | | | | |
|---|---|---|---|---|---|---|---|---|---|---|
| | | | Pre-filter | | | | Recovery (%) | | | |
| | | Dilution | HA | Protein | HA | Protein | | p vs | | p vs |
| Samples | | factor | (μg/m) | (μg/ml) | (μg/ml) | (μg/ml) | HA | undiluted | Protein | undiluted |
| MAU/ | Undiluted | — | 273 ± 61 | 84 ± 13 | 246 ± 39 | 69 ± 2 | 91 ± 6 | N/A | 83 ± 10 | N/A |
| saline | Diluted | 1.5 | 182 ± 41 | 56 ± 9 | 150 ± 25 | 46 ± 3 | 83 ± 5 | 0.278 | 83 ± 8 | 0.958 |
| | | 2.0 | 136 ± 30 | 42 ± 7 | 146 ± 29 | 35 ± 3 | 100 ± 2 | 0.058 | 85 ± 6 | 0.847 |
| | | 2.5 | 109 ± 24 | 34 ± 5 | 106 ± 1 | 29 ± 5 | 99 ± 21 | 0.337 | 87 ± 1 | 0.627 |
| MAU/ | Undiluted | — | 225 ± 35 | 54 ± 5 | 215 ± 0.4 | 39 ± 6 | 96 ± 1 | N/A | 72 ± 3 | N/A |
| WFI | Diluted | 1.5 | 150 ± 23 | 36 ± 4 | 140 ± 16 | 27 ± 6 | 93 ± 10 | 0.811 | 77 ± 8 | 0.605 |
| | | 2.0 | 112 ± 18 | 27 ± 3 | 106 ± 4 | 24 ± 6 | 95 ± 4 | 0.792 | 89 ± 10 | 0.283 |
| | | 2.5 | 90 ± 14 | 22 ± 2 | 87 ± 9 | 22 ± 6 | 96 ± 11 | 0.939 | 100 ± 14 | 0.238 |

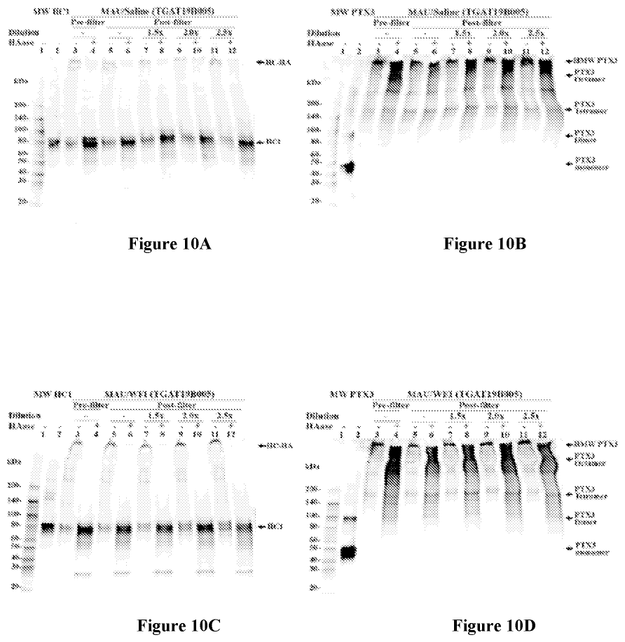

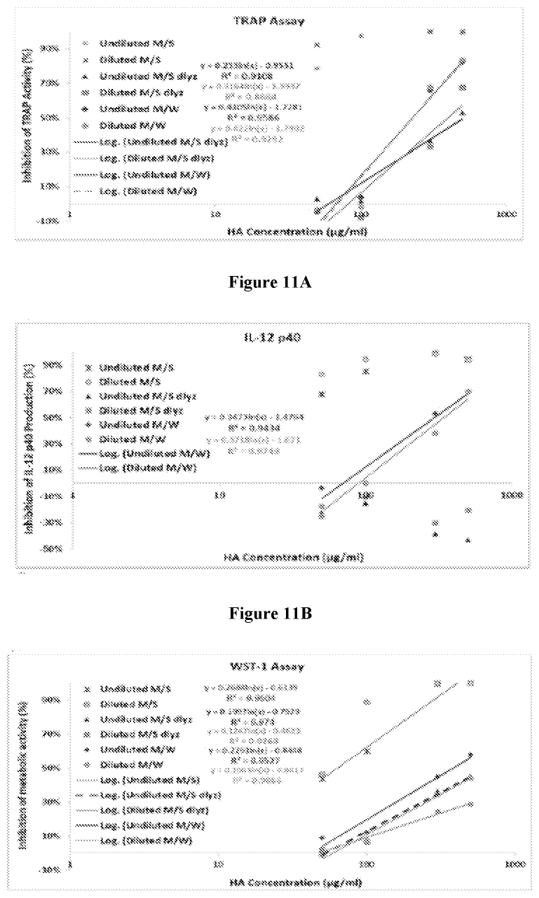

Agarose gel analysis of MAU with or without dilution after filtration showed that BMW HA in a similar amount is present in all filtered MAUs and diluted MAUs (1.5- to 3-fold) as compared to unfiltered MAUs (FIG. 9), suggesting that HMW HA is preserved in MAUs after filtration and dilution (1.5- to 3-fold). Similarly, the Western blot analysis of MAU with or without dilution showed the presence of HC-HA/PTX3 in all the filtered MAUs and increased after dilution (FIGS. 10A-10D), suggesting that HC-HA/PTX3 is also preserved after filtration and increased after dilution. Collectively, filtration sterilization is feasible for MAU terminal sterilization. Filtration of diluted MAU by 2- to 2.5-fold is better than filtration of undiluted MAU by increasing the speed and the recovery of HMW HA and HC-HA/PTX3 while not impact on the recovery rate of HA and total proteins.

### Example 7. Filtration Sterilization and Dilution Effect on Protein Recovery

Because MAU needs lyophilization to obtain a higher concentration of HA that is then amenable for ODI-TRAP, dialysis against water is necessary as a step for the method suitability for MAU/saline to remove salt, which interferes with TRAP assay. Previous data have shown that dialysis of MAU/saline against water with or without 0.5 mM PMSF for 48 h could completely remove salt. However, a longer time of dialysis might cause degradation of HA and HC-HA/PTX3 with or without PMSF as suggested by agarose and Western blot analysis.

AM and UC from one donor was cryopulverized, extracted with saline at 1:4 (w/v) ratio at 4° C. for 1 h, centrifuged at 48,000 g for 30 min at 4° C. and the supernatant was collected as MAU/saline.

MAU/saline was diluted (2-fold) with saline. Both undiluted and diluted MAU/saline was sequentially filtered with a 0.45 μm filter and a 0.2 μm filter.

Filtered MAU/saline with or without dilution were dialyzed with 3.5k MWCO Slide-A-Lyzer G2 dialysis cassettes against water at 4° C. for 1, 3, 6, or 24 h without PMSF using the dialysis buffer (water) volume ≥200 times the volume of the sample and with a change of water every one hour until overnight for 24 h. Dialyzed MAU/saline was tested with the Chloride assay to determine the efficiency of salt removal by dialysis.

Table 6 summarizes the extent of salt removal by dialysis at different dialysis times. The result showed that dialysis for 3 h removed 99% chloride from both undiluted and diluted MAU/saline, while dialysis for 6 h reached the same desalting effect as dialysis for 24 h. Therefore, it was determined that dialysis time can be reduced to 3 h and dialysis for 3-6 h is acceptable for effective desalting of MAU/saline.

TABLE 6
Chloride concentration in MAU/saline before and after dialysis

| | | Undiluted MAU/saline | | | Diluted MAU/saline | | |
|---|---|---|---|---|---|---|---|
| | | Chloride | Removal of | p value | Chloride | Removal of | p value |
| MAU/saline | | (mg/dl) | chloride (%) | vs 24 h | (mg/dl) | chloride (%) | vs 24 h |
| Non-dialyzed | | 548.8 ± | / | / | 500.5 ± 17 | / | / |
| Dialyzed | 1 h | 22.7 ± 1.9 | 95.9 | 0.039 | 32.3 ± 2.1 | 93.5 | 0.028 |
| | 3 h | 4.2 ± 0.4 | 99.2 | 0.026 | 4.6 ± 0.1 | 99.1 | 0.005 |
| | 6 h | 1.2 ± 0.2 | 99.8 | 0.189 | 1.3 ± 0.03 | 99.7 | 0.210 |
| | 24 h | 0.8 ± 0.1 | 99.9 | / | 0.9 ± 0.2 | 99.8 | / |

The method suitability for MAU/water or MAU/saline with or without 2-fold dilution was compared among three potency assay(s), i.e., ODI-TRAP assay, M2 assay, an NO assay and/or a WST-1 assay.

AM and UC from the same donor was cryopulverized, extracted in saline or WFI at 1:4 (w/v) ratio at 4° C. for 1 h, centrifuged at 48,000 g for 30 min at 4° C. and the supernatant was collected as MAU/saline and MAU/WFI. MAU/saline and MAU/WFI were undiluted or diluted (2-fold) with respective excipient and filtered sequentially with a 0.45 μm filter and a 0.2 μm filter. Filtered MAU/saline was undialyzed or dialyzed against water at 4° C. for 3-6 h to remove salt.

All MAU/saline and MAU/WFI samples were tested for HA assay. MAU/saline and MAU/WFI were lyophilized or unlyophilized and tested in ODI-TRAP assay, M2 assay, an NO assay and/or a WST-1 assay with the volume of cell culture medium set at 100 μl for all three assays. The cell morphology was recorded by microscopic images. The lyophilized samples were tested at the HA dose of 50, 100, 300 and 500 μg/ml in each assay. The unlyophilized samples could only be tested at one HA dose of 50 μg/ml because a higher dose of HA (e.g., ≥100 μg/ml) resulted in disproportional less culture medium volume at 100 μl, rendering the cell assay invalid.

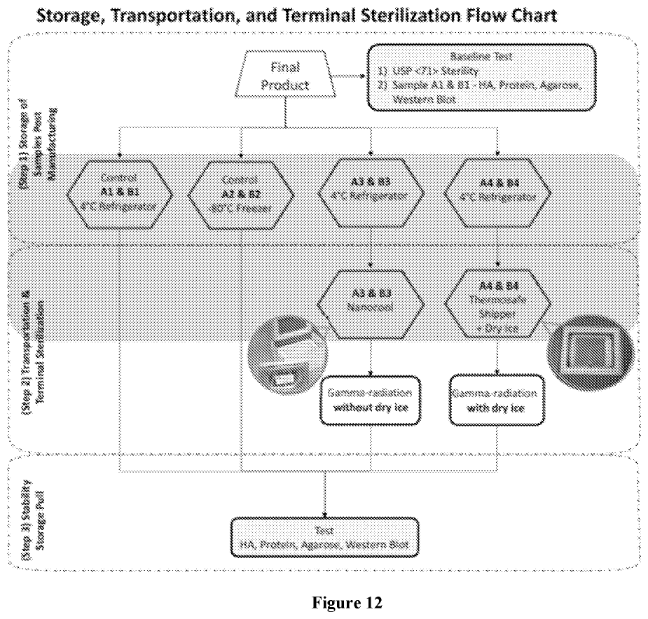

Table 7 summarizes the samples that exhibits dose-dependent linearity for the three assays while FIG. 12 illustrates the dose-dependent linearity for the three assays. The results suggested that:

1. Without lyophilization the highest concentration of HA that can be tested is 50 ug/ml, which still results in cell damage, thus still not suitable for all three assays.

2. Dialysis of MAU/saline is necessary because without dialysis cells exhibit morphological changes and cell toxicity due to high salt in all three assays.

3. The lowest detection dose is: a. TRAP:≥300 μg/ml HA for dialyzed MAU/saline and MAU/WFI with or without dilution. b. WST-1:≥100 μg/ml HA for dialyzed MAU/saline and MAU/WFI with or without dilution. c. M2:≥300 μg/ml HA for MAU/WFI with or without dilution. M2 is not suitable for dialyzed MAU/saline.

4. MAU/WFI with or without dilution is more potent than MAU/saline in all three assays, i.e., to exert greater extent of inhibition on TRAP, WST-1 and IL-12p40 or promotion on IL-10 at the same dose of HA and to exhibit better linearity.

5. Diluted MAU/WFI performed better than Undiluted in WST-1 and M2 with better linearity. Diluted performed equivalently to undiluted in TRAP with similar linearity. For dialyzed MAU/saline, Undiluted performed better than Diluted in TRAP with better linearity and Undiluted performed equivalently to Diluted in WST-1 with similar linearity.

6. For MAU/WFI with or without dilution, on lowest detection dose, WST-1 performed better than TRAP, which performed equivalently to M2. On linearity, with Dilution WST-1 performed equivalently to M2, which performed better than TRAP. For Undiluted, WST-1 performed equivalently to TRAP and M2. For, dialyzed MAU/saline with or without dilution, on lowest detection dose and linearity WST-1 performed better than TRAP.

TABLE 7
Comparison of MAU samples exhibiting dose-
dependent linearity in three potency Assays

| | Correlation Coefficient (R²)/ | | | |
|---|---|---|---|---|
| | Lowest Detection Dose (LDD) | | | |
| | | | M2 | |
| Samples | TRAP | WST-1 | (IL-12 p40) | Superiority of Assays |
| Dialyzed | 0.9108/ | 0.974/ | / | Linearity and LDD: |
| MAU/saline | 300 μg/ml | 100 μg/ml | | WST-1 > TRAP |
| Undiluted | | | | |
| Dialyzed | 0.8664/ | 0.9768/ | / | Linearity and LDD: |
| MAU/saline | 300 μg/ml | 100 μg/ml | | WST-1 > TRAP |
| Diluted | | | | |
| MAU/WFI | 0.9586/ | 0.9527/ | 0.9434/ | Linearity: WST-1 = TRAP = M2 |
| Undiluted | 300 μg/ml | 100 μg/ml | 300 μg/ml | LDD: WST-1 > TRAP = M2 |
| MAU/WFI | 0.9292/ | 0.9866/ | 0.9749/ | Linearity: WST-1 = M2 > TRAP |
| Diluted | 300 μg/ml | 100 μg/ml | 300 μg/ml | LDD: WST-1 > TRAP = M2 |

### Example 8: Effect of Processing Parameters on Fetal Support Tissue Product

A study was conducted testing the following process parameters for producing a morselized amniotic membrane and umbilical cord fetal support tissue fetal support tissue product: 1) storage temperature; 2) storage time; 3) excipient for morselization, i.e., saline or WFI; and 4) terminal sterilization (Gamma-irradiation with or without dry ice).

Methods

The following process controls were applied: (1) Placenta—amniotic membrane (AM) and umbilical cord (UC)— donors used were human transplantable grade with USP<61> acquisition culture reporting 0 colony-forming unit (CFU); (2) supplies were single use and sterile to ensure no cross-contamination; (3) excipients used were only new reagents and it was ensured that the seal on cap was intact before use; (4) the final product container was sterile and RNase/DNase free to eliminate the possibility of contamination during storage; and (5) upon completion of filling and labeling, a final product sample was sent for USP<71> sterility testing to ensure there is no microorganism in the baseline product.

Tissue from the two donors (AM and UC, separately) was subdivided equally by wet weight into two groups (A and B, see Table 8) according to two excipients, i.e., Saline or WFI. The following ordered process steps were performed separately for each group with a new blender cup, conical tubes, 200 μm filter, sterile container and the correlating excipient: morselization in the excipient, centrifugation, filtration, formulation, packaging for distribution, and terminal sterilization. Centrifugation was performed for 30 min at room temperature and 3095 rcf (4000 rpm). All samples were stored according to Table 8.

A sample was taken from each group (A and B) and filled with NLT (No Less Than) ml into a separate sterile 15 mL Screw Cap Tube for Sterility USP<71> test at t=0. Aliquots were taken from each group (A and B) to generate four (4) subgroups (A1-4 and B1-4, see Table 8) to yield at least 13 samples per subgroup (13 samples per subgroup×4 subgroups×2 groups=total of 104 samples) and each sample had a minimal volume of 4.6 ml filled in a 15 mL Screw Cap Tube. 5 ml per tube were filled using the entire MAU preparation (i.e., more than 13 tubes). Labels used were weatherproof, containing sample ID and date of manufacturing. Further, all samples were stored according to Table 8. FIG. 12 provides a flow chart detailing the storage, transportation, and terminal sterilization steps used.

TABLE 8
Sample Storage Protocol

| | | | | Transportation and | Transportation and | Total numbers |
|---|---|---|---|---|---|---|
| | Morselized | | | Gamma-irradiation | Gamma-irradiation | of samples per |
| Subgroup | with | Storage | | without dry ice | with dry ice | subgroup |
| A1 | Saline | 4° | C. | − | − | 13 |
| A2 | | −80° | C. | − | − | |
| A3 | | 4° | C. | + | − | |
| A4 | | 4° | C. | − | + | |
| B1 | WFI | 4° | C. | − | − | 13 |
| B2 | | −80° | C. | − | − | |
| B3 | | 4° | C. | + | − | |
| B4 | | 4° | C. | − | + | |

At the baseline (t=0) three (3) sterility USP<71> samples were shipped to contract laboratory VRL for testing. The samples were shipped in a VRL shipper filled with ice packs. For Group A3 and B3, samples were packaged in a Nanocool shipper that had been validated to maintain 8° C. for 48 to 92 hours. For Group A4 and B4, samples were packaged in Thermosafe Shipper with NLT 30 lbs of dry ice (outside) and the Corepack. The above samples were delivered by contract carrier (i.e. FedEx). To monitor the temperature maintained, a data logger was placed in the shipping container of Group A3 and B3. The data logger was removed during irradiation and returned to the shipping container after irradiation. The data from the data logger was analyzed to exhibit the temperature during shipping. Dry ice sublimates at −78.5° C. and hence for Group A4 and B4, the −80° C. temperature control for samples was judged by visual inspection for the presence of dry ice and the residual dry ice was weighed after shipping for information only. Gamma irradiation was performed by Sterigenics at a dose of 25±10% kGy according to the scheduling below.
Monday—Shipment of samples
Tuesday—Receipt of shipment and Gamma-irradiation process
Wednesday—Completion of Gamma-irradiation and process paperwork for return.
Thursday—Return of samples

The samples were subject to analytical testing, including sterility USP<71> testing, where one sample per Group (A, and B) was tested immediately after manufacturing before storage, i.e., t=0. The samples were subject to BCA, HA, Agarose gel analysis, Western Blot and ODI-TRAP tests. Three samples, from each subgroup (n=3) of A and B, were subjected to these tests at t=0, and three samples from each subgroup (n=3) of A1/A2 and B1/B2 were subjected to these test at t=1, 3 and 6 months. Additionally, three samples from each subgroup (n=3) were subjected to these tests at t=1, 3, and 6 months after samples having been irradiated, shipped back, and stored in the correlating temperature, as specified by subgroups A3/A4 and B3/B4.

Results

The sterility USP<71> testing produced the following results:
Group A<71> no growth-final.
Group B<71> no growth-final.

Regarding the HA analysis, for Group A (saline), at t=3 m, A1 and A2 (4° C. or −80° C. respectively, control, without Gamma-irradiation) showed no difference in HA concentration compared to the baseline and between each other, while A4 (post Gamma-irradiation with dry ice) HA concentration was undetectable, as seen at t=1 month (Table 9). For Group B (WFI), B1 and B2 (4° C. or −80° C. respectively, control, without Gamma-irradiation) showed no difference in HA concentration compared to the baseline and between each other, while B4 (post Gamma-irradiation with dry ice) showed a significant reduction compared to the baseline and compared to the control (B2, Table 9), this is consistent with the reduction trend seen in the t=1 m.

Regarding the total protein analysis: for Group A (saline), at t=3 m the protein concentration in both A1 and A2 (4° C. or −80° C. respectively, control, without Gamma-irradiation) were undetectable, while the concentration was detectable at t=1 m and the baseline (Table 9). A4 (post Gamma-irradiation with dry ice) had no statistical difference compared to the baseline. For Group B (WFI), B1 and B2 (4° C. or −80° C. respectively, control, without Gamma-irradiation) were undetectable as seen in at t=1 m and the baseline. Sample B4 (after Gamma-irradiation with dry ice) protein was undetectable as seen in t=1 m (Table 9).

TABLE 9
HA and protein concentration in MAU Group A and B at t = 3 month (average ± standard deviation)

| MAU | t = 0 | Subgroups | | | p value | p value |
|---|---|---|---|---|---|---|
| Group A | (Baseline) | A | t = 1 m | t = 3 m | vs t = 0 | vs A1 |
| HA | 239.4 ± 16.7 | A1 | 215.0 ± 21.2 | 250.6 ± 21.8 | 0.606 | N/A |
| (μg/ml) | (218.5 ± 13.7) | | (183.2 ± 14.7) | (227.9 ± 19.3) | (0.621) | |
| | | A2 | 215.3 ± 8.0 | 257.0 ± 16.9 | 0.358 | 0.775 |
| | | | (183.8 ± 4.6) | (233.6 ± 15.1) | (0.369) | (0.776) |
| | | A3 | Undetectable | [1] | N/A | N/A |
| | | A4 | Undetectable | Undetectable | N/A | N/A |
| | | | (11.7 ± 2.4) | (10.4 ± 1.4) | (0.001) | (0.039) |
| Protein | 85.7 ± 5.6 | A1 | 69.1 ± 0.6 | Undetectable | N/A | N/A |
| (μg/ml) | (86.7 ± 3.6) | | (82.2 ± 0.4) | (73.0 ± 1.0) | (0.015) | |
| | | A2 | 79.6 ± 8.7 | Undetectable | N/A | N/A |
| | | | (89.0 ± 5.6) | (74.9 ± 1.9) | (0.018) | (0.364) |
| | | A3 | 621.6 ± 94.0 | [1] | N/A | N/A |
| | | A4 | 107.5 ± 1.5 | 27.2 ± 12.0 | 0.06 | N/A |
| | | | 107.4 ± 1.0 | (95.0 ± 6.9) | (0.311) | (0.134) |
| MAU | t = 0 | Subgroups | | | p value | p value |
| Group B | (Baseline) | B | t = 1 m | t = 3 m | vs t = 0 | vs B1 |
| HA | 216.0 ± 2.6 | B1 | 246.7 ± 0.07 | 160.9 ± 25.3 | 0.198 | N/A |
| (μg/ml) | (199.3 ± 2.2) | | (220.3 ± 0.1) | (151.1 ± 19.6) | (0.176) | |
| | | B2 | 261.6 ± 7.5 | 185.3 ± 7.6 | 0.09 | 0.392 |
| | | | (231.4 ± 5.7) | (170.0 ± 6.0) | (0.071) | (0.39) |
| | | B3 | Undetectable | [1] | N/A | N/A |
| | | B4 | 47.7 ± 4.0 | 25.5 ± 8.6 | 0.014 | 0.06 |
| | | | (70.8 ± 2.9) | (45.7 ± 7.1) | (0.014) | (0.029) |
| Protein | Undetectable | B1 | Undetectable | Undetectable | N/A | N/A |
| (μg/ml) | (30.1 ± 1.0) | | (Undetectable) | (31.0 ± 3.3) | (0.766) | |
| | | B2 | Undetectable | Undetectable | N/A | N/A |
| | | | (Undetectable) | (33.2 ± 0.7) | (0.027) | (0.513) |
| | | B3 | 481.3 ± 63.0 | [1] | N/A | N/A |
| | | B4 | Undetectable | Undetectable | N/A | N/A |
| | | | (Undetectable) | (44.8 ± 1.7) | (0.022) | (0.056) |

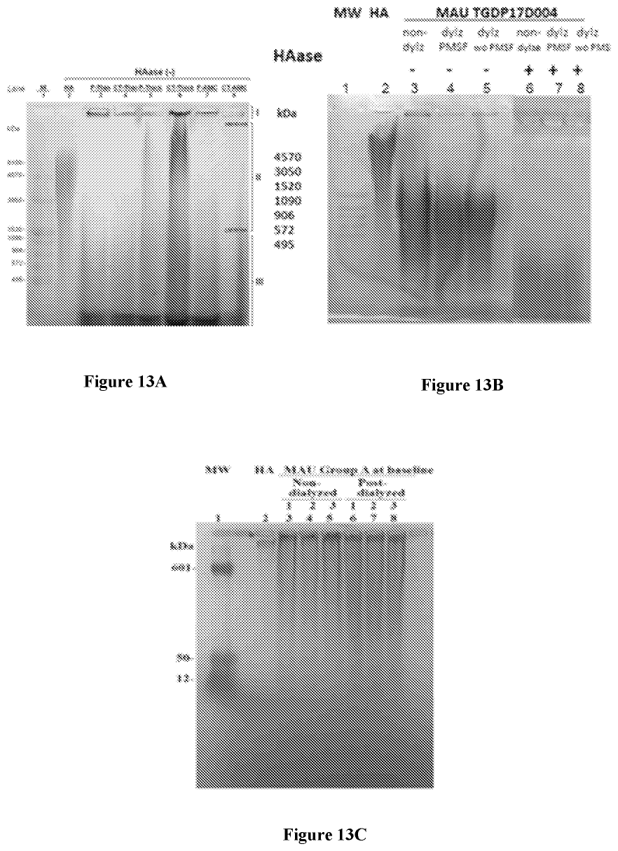

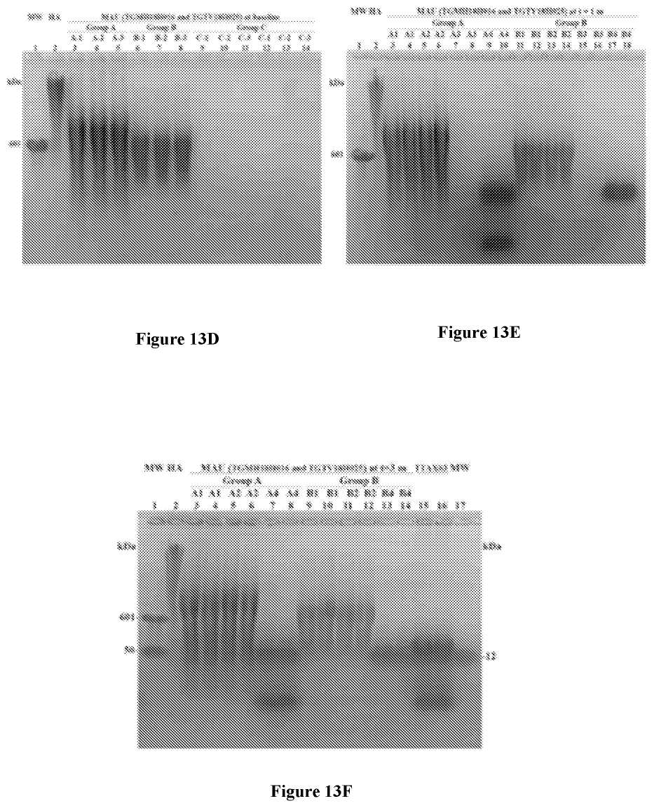

Regarding the agarose gel analysis (FIGS. 13A-13F), for each lane of A1, A2, B1 and B2 samples, the same amount of 10 μg HA was loaded. For each lane of A4 and B4 samples, the same volume as A1 or B1 at 10 μg HA respectively, was loaded due to the low A4 and B4 HA concentrations. Lane 1 has 50 and 601 kDa markers and lane 17 has a 12 kDa marker (the 50 and 12 kDa were separated for clearer distinction) (FIG. 13C). Both Groups A (saline, non-dialyzed) and B (WFI) at t=3 m control samples (without Gamma-irradiation) (A1, A2, and B1, B2, FIG. 13F, lanes 3-6 and lanes 9-12 respectively) showed the same HA distribution pattern as that seen in their correlating baseline samples (FIG. 13E, lanes 3-5 and lanes 6-8 for Groups A and B respectively) and the Gamma-irradiation with dry ice samples (A4 and B4) showed the same HA pattern (FIG. 13F, Groups A and B, lanes 7-8 and lanes 13-14 respectively) as was seen at t=1 month with the correlating samples (FIGS. 13B and 13E, Groups A and B, lanes 9-10 and lanes 17-18 respectively). Overall, the HA intensity at T=3 was weaker than that in T=1. Whether this is due to HA distribution change or gel staining difference remains unknown.

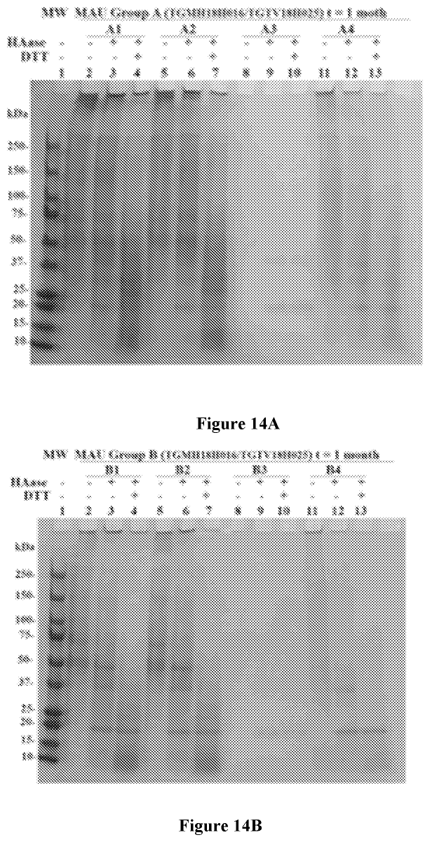

For the Coomassie blue stain analysis (FIGS. 14A-14B), for Group A (saline, FIG. 14A), 20 μg HA or the same volume was loaded in each loading well depending on whether there was measurable HA. For Group B (WFI, FIG. 14B), 40 μg HA or the same volume was loaded. When comparing control samples in Group A (A1 and A2) to the control samples in Group B (B1 and B2) both showed similar patterns, the same after HAase digestion. Following HAase and DTT treatments both Groups had a smear below 50 kDa to the bottom of the lane. For both Groups, no difference in the storage temperature (4° C. vs −80° C.) was noted. Post Gamma-irradiation without dry ice (A3 and B3) showed two bands at 37 and 20 kDa. Post Gamma-irradiation with dry ice (A4 and B4) and without treatment showed a smear form the top of the gel to ˜30 kDa for both. Following HAase digestion both Groups showed two bands at 20 kDa 37 kDa (surprisingly more noticeable in Group B) with a faint smear to the top of the gel. Following HAase and DTT treatments both Groups showed a 20 kDa band. Consistently Group B smears were fainter than A.

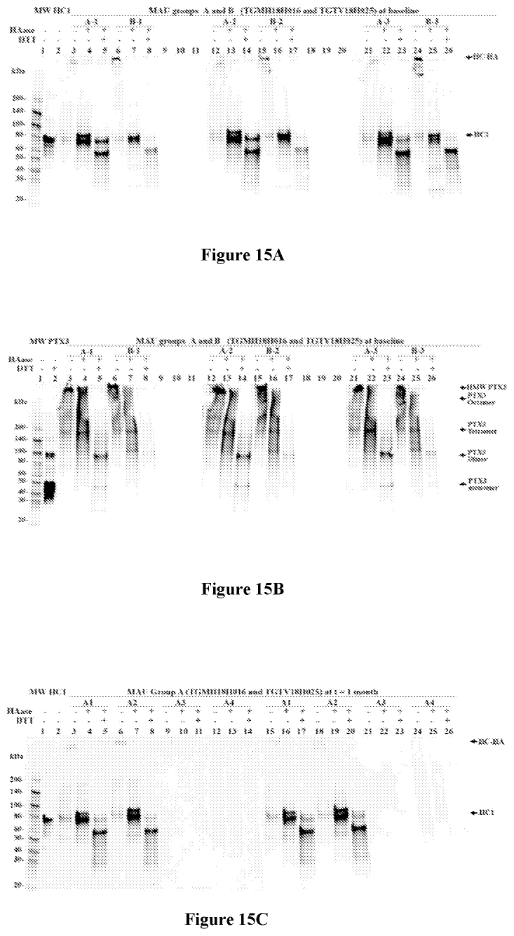

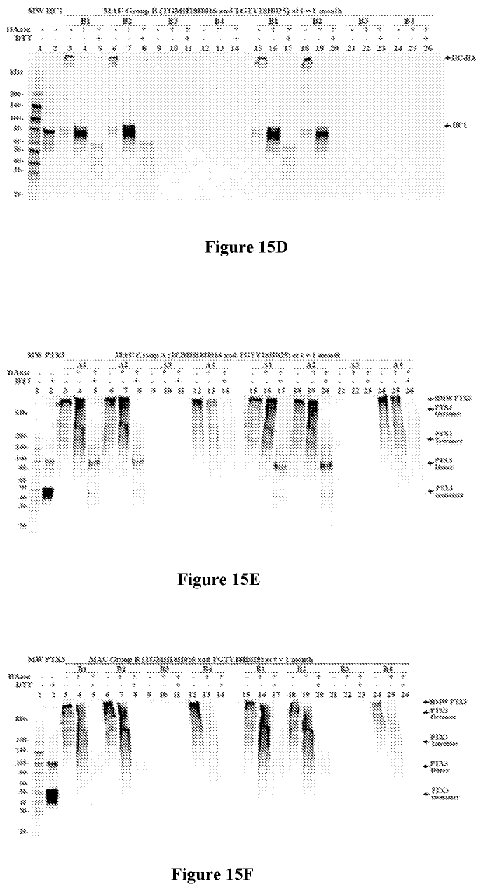

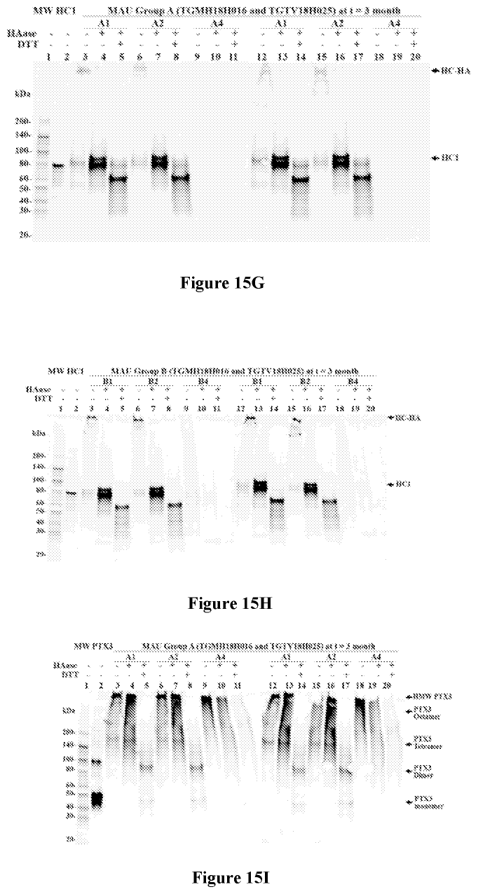

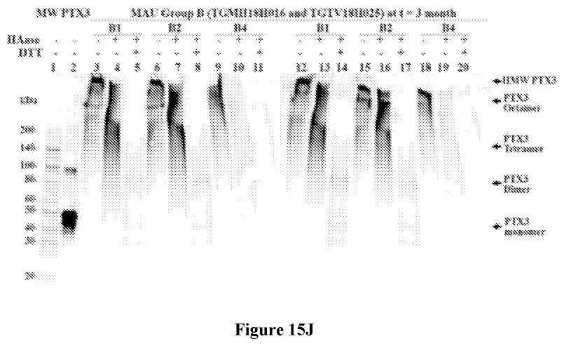

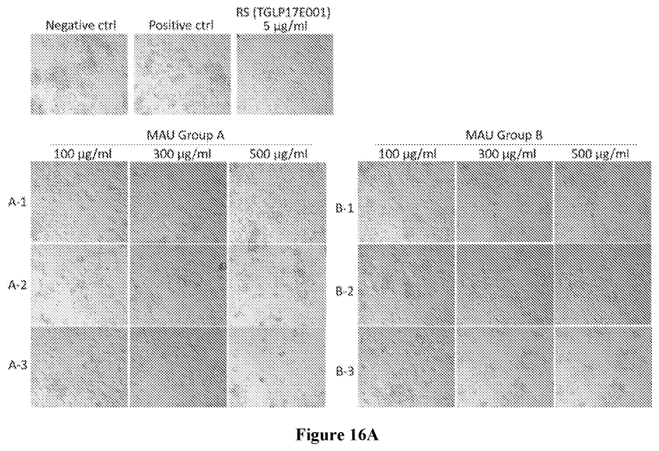

Regarding the Western Blot Analysis (FIGS. 15A-15J), samples of A1, A2, B1 and B2 were loaded at 10 μg HA in each loading well, and samples of A4 and B4 were loaded with the same volume as A1 or B1 at 10 μg HA, respectively. Samples without Gamma irradiation Groups A and B (A1, A2, B1 and B2) with or without HAase or HAase/DTT treatment showed the same HC1 (FIGS. 15G and 15H) and PTX3 (FIGS. 15I and 15J) patterns compared to the correlating samples at the baseline (FIGS. 15A and 15B) respectively. Samples with Gamma irradiation Groups A and B (A4 and B4) with or without HAase or HAase/DTT treatment showed the same HC1 (FIGS. 15G and 15H) and PTX3 (FIGS. 15I and 15J) patterns compared to the correlating samples at t=1 m (FIGS. 15C, 15D, 15E and 15F) respectively.

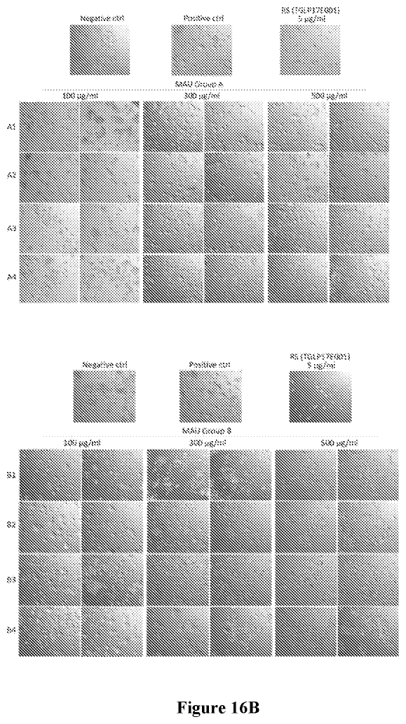

FIG. 16A shows cell morphology for MAU Groups A (saline) and B (WFI) at baseline (t=0). FIG. 16B shows cell morphology for MAU Groups A and B at t=1 month. The positive control showed multinucleated osteoclast formation compared to the negative control. The formation of multinucleated osteoclasts was inhibited by the Reference Standard (RS) loaded at 5 μg/ml HA (FIGS. 16A and 16B). MAU Groups A and B at baseline (FIG. 16A) and at t=1 month (FIG. 16B) significantly inhibited the multinucleated osteoclasts formation at 300 and 500 μg/ml HA.

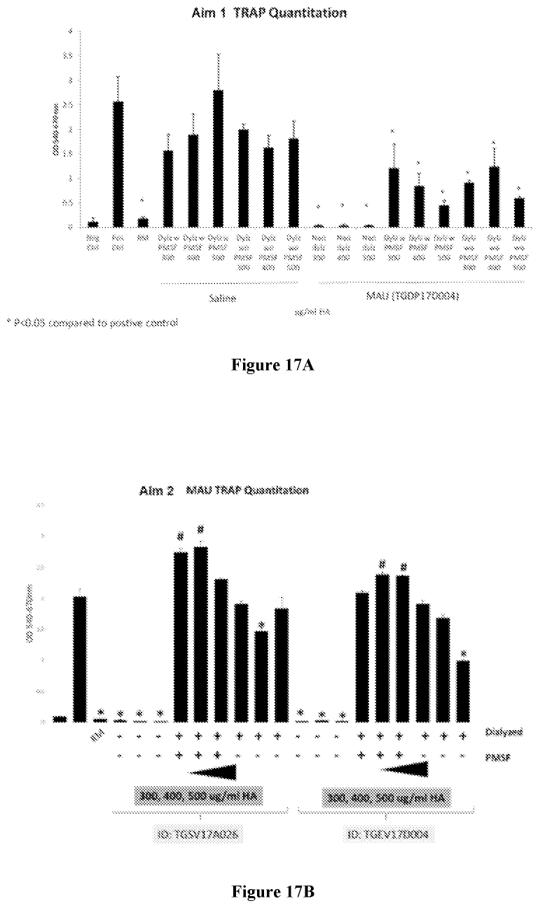

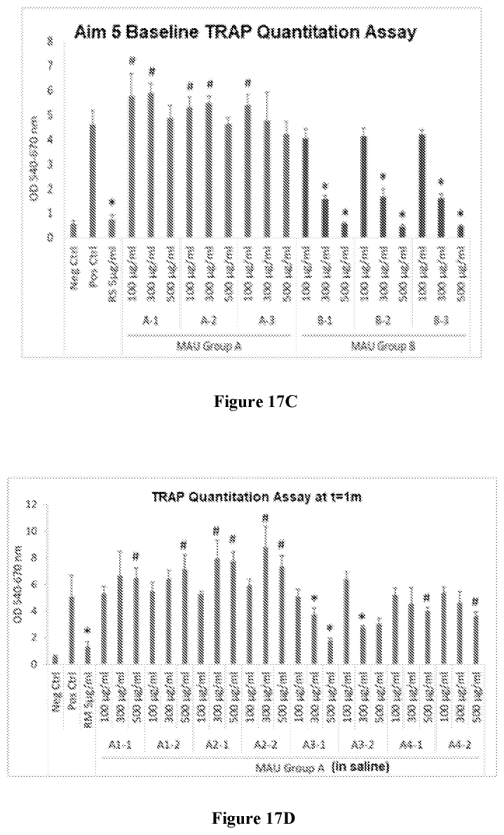

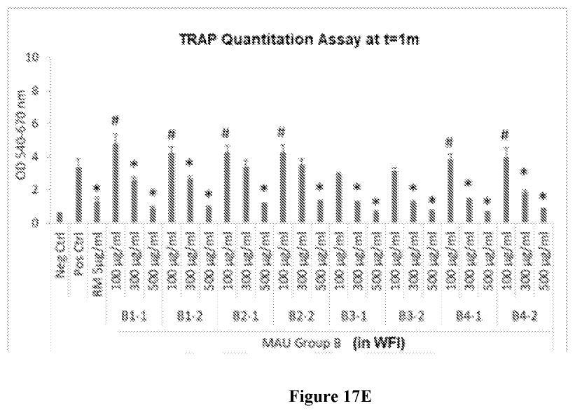

FIGS. 17A-17E show the results of the TRAP assays. The positive control showed high TRAP activity compared to the negative control (p<0.05) and the Reference Material (RM) loaded at 5 μg/ml HA significantly inhibited the TRAP activity (p<0.05), which validates the assay (FIG. 17A-17E). FIG. 17A, MAU (saline) dialyzed with or without PMSF significantly inhibited TRAP activity at all concentrations (p<0.05), while saline alone dialyzed with or without PMSF did not affect TRAP activity, confirming the removal of salt by dialysis. FIG. 17B, MAU (saline) dialyzed without PMSF showed inhibition of TRAP activity (p≤0.05) but showed promotion of TRAP activity when dialyzed with PMSF (p≤0.05) from both donors. FIG. 17C, MAU Group A (saline) showed promotion of TRAP activity at 100 and/or 300 μg/ml HA (p≤0.05) with no inhibition activity, while MAU Group B (WFI) showed significant inhibition of TRAP activity dose-dependently (p≤0.05) without promotion. FIG. 17D, MAU Group A (saline) stored at 4° C. (A1) or −80° C. (A2) showed promotion of TRAP activity at 300 and/or 500 μg/ml HA (p<0.05) with no inhibition activity. MAU Group A gamma-irradiated without dry ice (A3) showed inhibition of TRAP activity (p≤0.05) but gamma-irradiated with dry ice (A4) showed promotion of TRAP activity with no inhibition activity. FIG. 17E, MAU Group B (WFI) stored at 4° C. (B1) or −80° C. (B2) or gamma-irradiated without (B3) or with dry ice (B4) all showed inhibition of TRAP activity at 300 and/or 500 μg/ml HA (p≤0.05) with promotion of TRAP activity at 100 μg/ml HA in B1, B2 and B4 (p≤0.05). *p≤0.05 vs positive control, TRAP inhibition. #p<0.05 vs positive control, TRAP promotion.

### Analysis of Results

Regarding storage at 4° C. vs −80° C., there was stability regarding HA (quantity and quality) and HC-HA/PTX3 complex for control samples of Groups A (A1 and A2) and B (B1 and B2) with the following exception: HA concentration was stable in Groups A and B without the increase in HA concentration that was seen in Group B in t=1 m. HA was accompanied by chondroitin sulfate in Group A but not Group B as seen in the baseline and t=1 m. These results suggest HA was maintained during sample storage for 3 months, and that there is no difference between the storage temperature (4° C. vs −80° C.). The agarose gel results suggest that HA MW distribution in both Groups A and B had no changes with the storage for up to 3 months at 4° C. or −80° C., which is consistent with the HA assay result. Proteins in Groups A were undetectable at 4° C. and −80° C. For Group A the t=3 m results are not consistent with the t=1 m and the baseline but for Group B the undetected protein concentration was consistent with the t=1 m and the baseline results. Groups A and B maintained the HA, HC-HA, HC1 and PTX3 patterns according to the correlating treatments seen in the baseline, supporting the stability in three months and indicating the presence of the HC-HA/PTX3 complex.

Regarding the gamma-irradiation, without dry ice (A3 and B3), protein bands (tested on t=1 m) were markedly reduced as analyzed by Coomassie blue. Agarose gel of the samples did not show any HA, suggesting the completed degradation of HA by Gamma-irradiation (compared to the control). Additionally, the Western Blot did not show HC1 or PTX3, indicating that HC-HA/PTX3 complex was not preserved. With dry ice, saline (A4) shows that HA was undetectable, but the protein concentration had maintained the concentration as seen in t=1 m. WFI (B4) HA concentration was reduced by 87.2% compared to the baseline and the protein concentration remains undetectable as seen at t=1 m and the baseline. Agarose gel results post Gamma-irradiation results are consistent with the correlating samples at the t=1 month indicating the stability. Comparing the Western Blot samples (A4 and B4, t=3 m) to the samples at t=1 m, in both cases no HC-HA, no HC1 and HMW PTX3 with the correlating treatment was detected, suggesting no HC-HA/PTX3 complex in samples which complies with the t=1 m results.

Regarding the TRAP assays, at t=1 m, saline samples without gamma-irradiation (A1 and A2) showed TRAP promotion activity as seen at the baseline. After Gamma-irradiation with or without dry ice (A3 and A4) showed inhibition activity not seen in the at the baseline. At t=1 m WFI samples without Gamma-irradiation (B1 and B2) showed TRAP inhibition activity as seen in the control and at the baseline. For Group B, using the polynomial 4 calculation revealed protein concentrations at the baseline (not seen before) and at t=3 m but not in t=1 m.

Accordingly, it can be concluded that control samples (A1, A2, B1 and B2) preserve the HC-HA/PTX complex at t=3 m and as seen at t=1 m and the baseline. Post Gamma sterilization (with dry ice) samples (A4 and B4) did not preserve the HC-HA/PTX complex at t=3 m as seen in t=1 m. Post Gamma samples (A3, A4, B3 and B4) did not show HC-HA without treatment or HC1 after HAase and DTT reduction, but did show BMW PTX3 in A4 indicating the HC-HA/PTX complex was not preserved at t=1 m.

### Example 9: Stability Testing of MAU Product

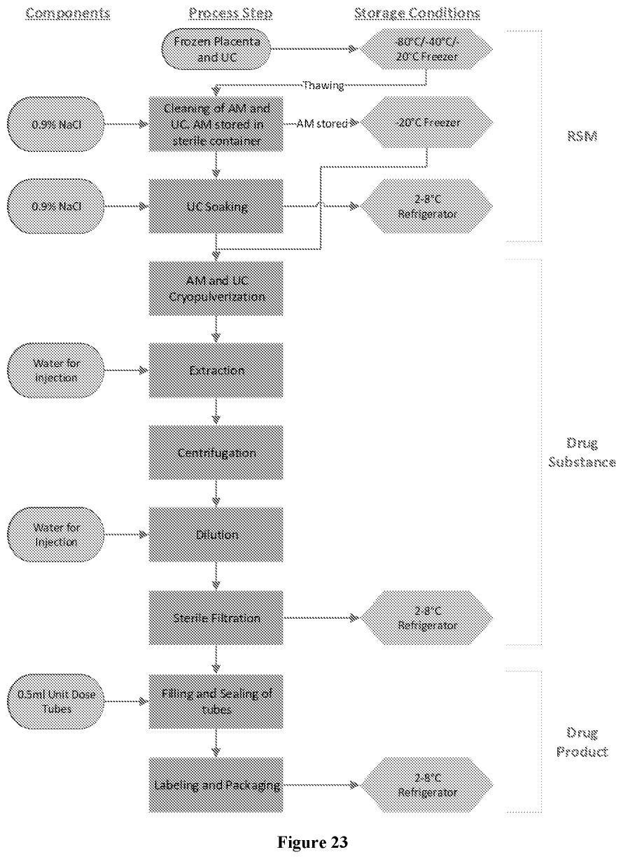

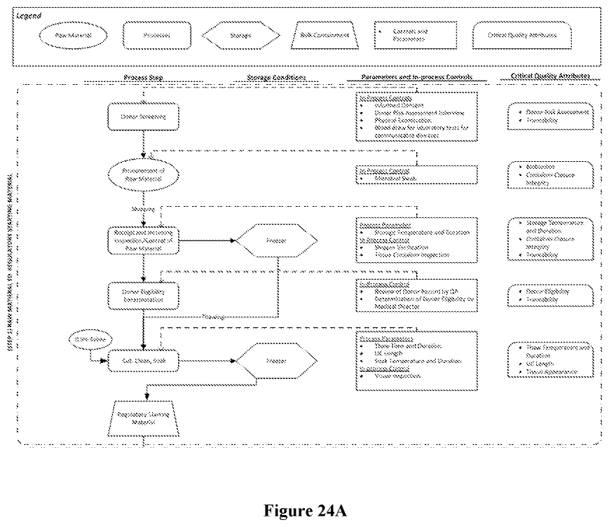

MAU was manufactured using the following process described below and illustrated in FIG. 23. The final product was tested for stability using HA, ODI-TRAP, WST-1, NO and M2 assays.

Step 1: Regulatory Starting Material

Processing Tissue from receipt to regulatory starting material. The tissue preparation process for regulatory starting material included cleaning, cutting and soaking steps.

Step 2: Drug Substance

Cryopulverization. AM and UC tissues were thawed if previously stored. The tissue was weighed and transferred into a freezer mill vial. The closed vial was transferred to the freezer mill where the tissue was cryopulverized. Once the cycle was completed, the pulverized tissue was transferred to a sterile bottle (extraction bottle). The total pulverized tissue was weighed.

Extraction. WFI (Water for Injections) was added to a bottle containing the pulverized tissue at a 1:4 w/v tissue (gram):WFI (milliliter) ratio. The filled bottle was sealed and transferred for extraction to a bottle rotator located inside a 2-8° C. refrigerator. Extraction will ran for 60±5 minutes at 12±6 rpm.

Centrifugation. Immediately after extraction, the solution was filled into centrifuge tubes for centrifugation. The centrifuge was run for 30 minutes at 14,000 g. The supernatant was collected and the pellet was discarded.

Dilution. The supernatant collected from centrifugation was diluted with WFI to a final volume of 500 ml. The container with the 500 ml diluted solution was sealed and transferred to the tube rotator located inside a 2-8° C. refrigerator. The solution was mixed for 30±3 minutes at 12±6 rpm.

Sterile Filtration. Sterile filtration was performed using a peristaltic pump and a filtration assembly. The filtration assembly was sterilized by gamma radiation. The diluted supernatant was filtrated at NMT 18 psi from the dilution bottle to a sterile filtrate bag (closed system), which is part of the filtration assembly, using a capsule filter equipped with a 0.65 micron removal rating asymmetric layer on the upstream side and a 0.2 micron removal rating symmetric layer on the downstream side.

Step 3: Drug Product

Filling, Sealing, Labeling, Packaging and Storage. This process includes filling of filtered drug substance into final product tubes, sealing, labeling and storage of final product.

Furthermore, the assembly will be tested for system integrity (pressure decay), visual appearance and fluid path cleanliness as part of routine in-process controls implemented for

Unit dose tubes will be sealed. After sealing of the UDT, they will be moved outside of the cleanroom to a controlled, non-classified room where tube labeling and secondary packaging and sealing will take place.

Once filled and sealed tubes have been found acceptable by leak testing, they are labeled with the Finished Drug Product (FDP) batch number and packaged. Labels are visually inspected for visual defects and accuracy and labels are reconciled. Representative sampling will be performed for product release testing that includes sterility, endotoxin, identity, purity and potency, total fill volume and drop uniformity testing.

The final product was separated into three lots. The results of the USP<71> sterility tests are as follows:
D1—TGEA19I010—result was no growth.
D2—GJ19C018RBI—result was no growth.
D3—TGED19I007—result was no growth.

Table 10 shows the HA concentrations measured for each of the donors. Between the donors HA concentration was shown to be significantly (p<0.05) different when using the t-test, reviling that D1 and D2 had lower HA concentrations compared to D3.

TABLE 10
Donor HA Concentration

| TTBT01 | D1 | D2 | D3 |
|---|---|---|---|
| HA (μg/ml) | 32.6 ± 1.2 | 15.2 ± 0.6 | 48.3 ± 0.9 |
| p value vs Donor 3 | 1.4425E−10 | 2.00123E−13 | N/A |

Results

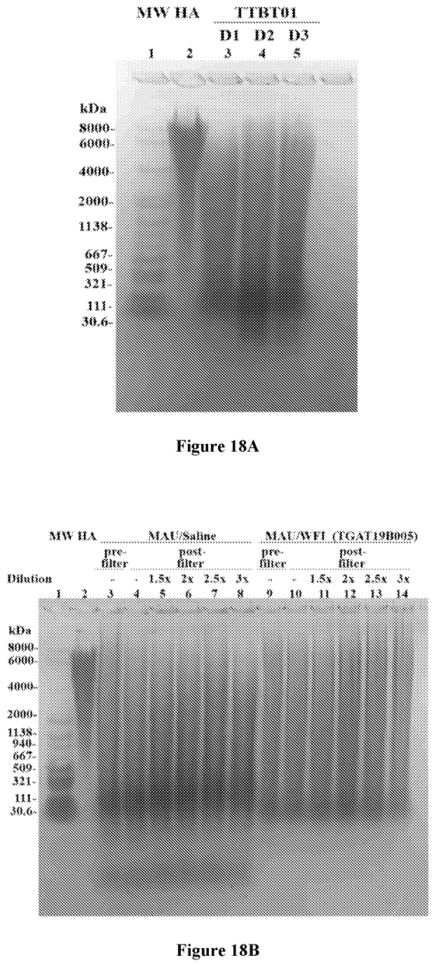

Agarose gel analysis (FIGS. 18A-18B) shows HMW HA present in all samples, with HMW>8000 kDa in D2 and D3 and at 6000 kDa in D1. A LMW band in purple (<30.6 kDa) present mainly in D2 more than D3, but very weak in D1, which is not noted in previous MAU/WFI (FIG. 18B, lanes 9-14).

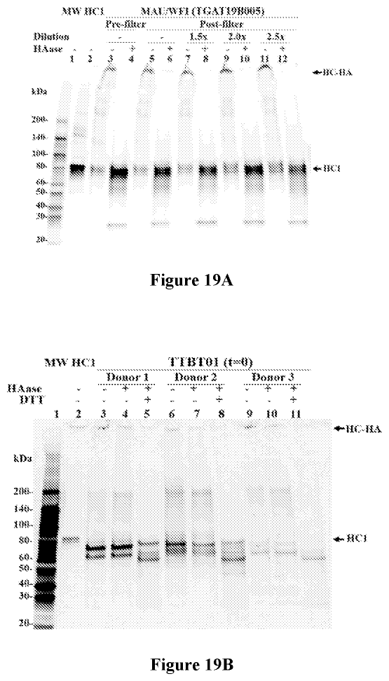

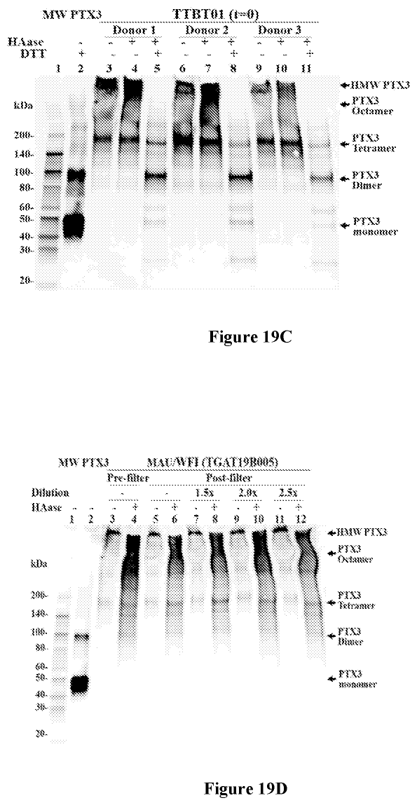

The Western blot analysis (FIGS. 19A-19D) for HC1 did not show dissociation of HC-HA because of the abundant presence of free HC1 before treatment and after HAase treatment. All samples were shown to have BMW PTX3, of which dissociation by HAase treatment is not obvious presumably due to the abundant presence.

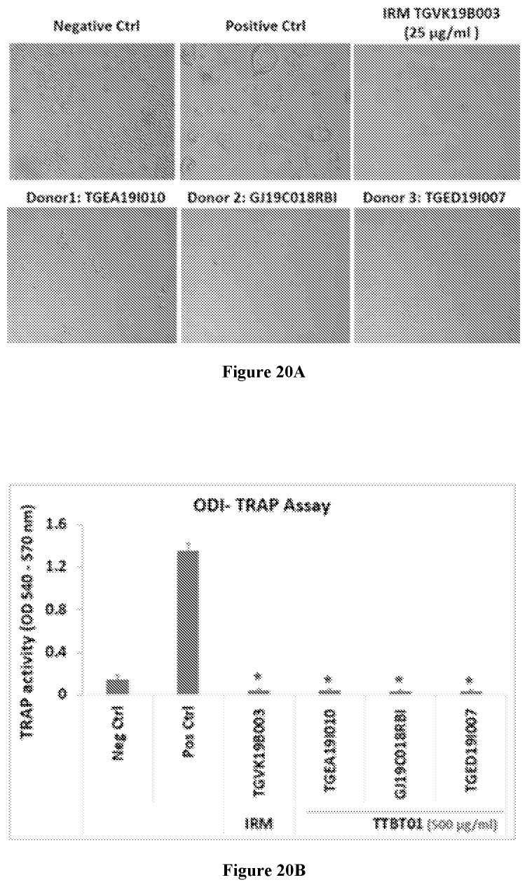

Regarding the ODI-TRAP assay (FIGS. 20A-20B), IRM (HC-HA/PTX3) (25 μg/ml) inhibited TRAP activity with inducing cell death. Additionally, samples (t=0) from all 3 donors inhibited TRAP activity at 500 μg/ml with cell death.

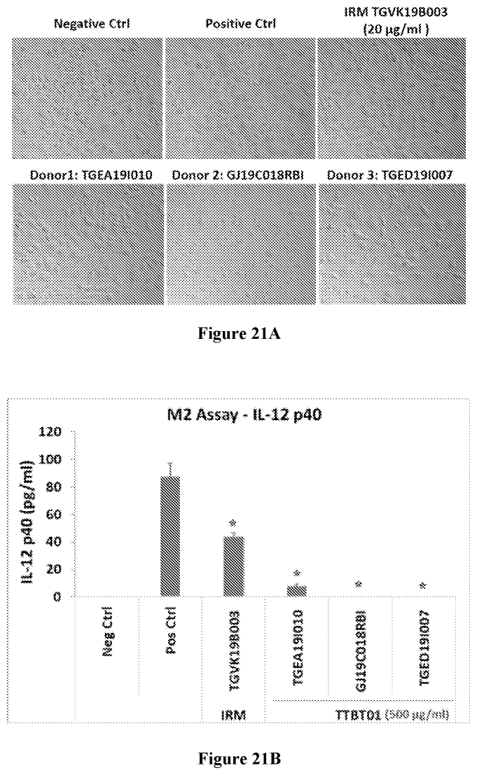

Regarding the WST-1 assay (FIGS. 21A-21B), IRM (25 μg/ml) inhibited cell metabolic activity and D1 and D3 inhibited cell metabolic activity at 500 μg/ml. D2 inhibitory effect resulted in the negative OD, so a deviation was initiated.

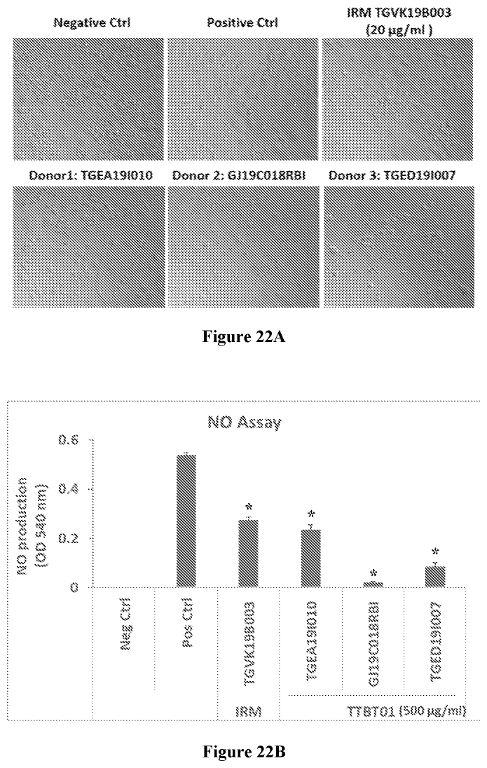

Regarding the M2 assay (FIGS. 22A-22B), IRM (20 μg/ml) downregulated IL-12 p40 production, and the 3 donors downregulated IL-12 p40 production at 500 μg/ml.

Regarding the NO assay (FIGS. 23A-23B), IRM (20 μg/ml) inhibited NO production, and the 3 donors inhibited NO production at 500 μg/ml with Donor 2 and 3 induced cell death.

Analysis

Similar to IRM (HC-HA/PTX3), all three TTBT01 donors showed inhibition on ODI-TRAP Assay, WST-1 Assay, M2 Polarization IL-12 Assay and NO Assay at the loading dose of 500 μg/ml of HA.

### Example 10: Follow-On Stability Testing of MAU Product

The MAU samples manufactured according to Example 9, are subjected to repeated HA, ODI-TRAP, WST-1 and M2 assays and the t=1, 2, and 3 time points, as is illustrated in Table 11. The data will then be tracked to determine the percentage change at the pull time points. All batches will be analyzed in the same manner during the stability study.

TABLE 11
Assay Strategy for MAU Formulation at Three Timepoints

| | | −20° C. Freezer | | | | | Refrigerator | | | |
|---|---|---|---|---|---|---|---|---|---|---|
| | | (maintained between −25° C. to −10° C.) | | | | | (maintained between 2° C. to 8° C.) | | | |
| | Acceptance | Initial | | | | | | | | |
| Tests | Criteria | (T = 0) | 1 | 2 | 3 | Reserved | 1 | 2 | 3 | Reserved |
| HA | To be | 36 mL | 36 mL | 36 mL | 36 mL | 2 × 36 mL | 36 mL | 36 mL | 36 mL | 2 × 36 mL |
| Assay | established | | | | | | | | | |
| | based on | | | | | | | | | |
| | initial, T = 0 | | | | | | | | | |
| | data² | | | | | | | | | |
| ODI- | To be | | | | | | | | | |
| TRAP | established | | | | | | | | | |
| | based on | | | | | | | | | |
| | initial, T = 0 | | | | | | | | | |
| | data² | | | | | | | | | |
| M2 (IL- | N/A (Report | | | | | | | | | |
| 12 p40) | as Found³) | | | | | | | | | |
| WST-1 | N/A (Report | | | | | | | | | |
| | as Found³) | | | | | | | | | |
| Agarose | N/A (Report | | | | | | | | | |
| gel⁵ | as Found³) | | | | | | | | | |
| Western | N/A (Report | | | | | | | | | |
| blot⁵ | as Found³) | | | | | | | | | |
| Nitric | N/A (Report | | | | | | | | | |
| Oxide | as Found³) | | | | | | | | | |
| (NO)⁵ | | | | | | | | | | |
| Sterility | No Growth⁴ | 1 mL | 0 | 0 | 1 mL | 2 × 1 mL | 0 | 0 | 1 mL | 2 × 1 mL |

### Example 12: Scale Up Manufacturing of MAU

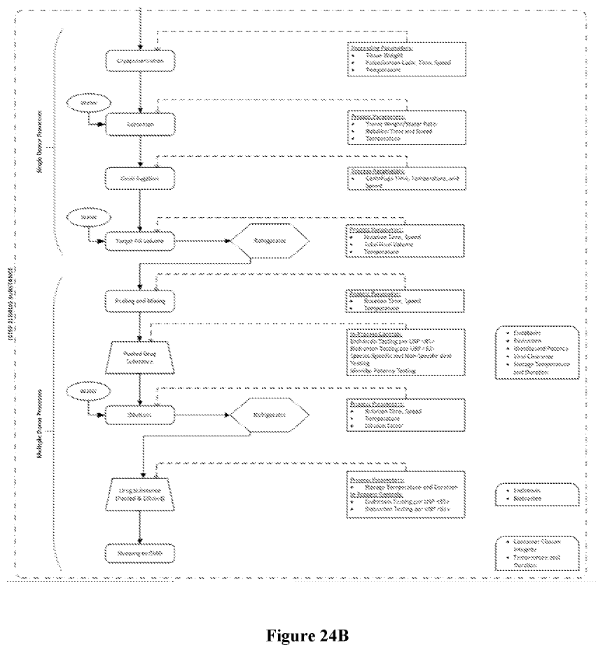

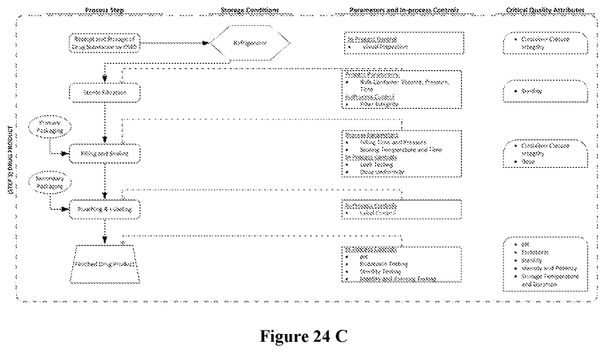

MAU is manufactured using the following process described below and illustrated in FIG. 24.

Step 1: Raw Material to Regulatory Starting Material. This step covers from the acquisition of tissue raw material (i.e., human birth tissue) to the production of the Regulatory Starting Material (RSM), which consists of human amniotic membrane (AM) and umbilical cord (UC) tissue from an individual donor. This step consists of donor screening, procurement, receipt and inspection, donor eligibility determination, tissue cleaning, cutting, and soaking, to generate the RSM.

Step 2: Drug Substance. Single Donor Processes and Multiple Donor Processes. This step consists of the cryopulverization, extraction, centrifugation, and target fill volume for individual donors. After sufficient number of donors are achieved, multiple donors are pooled together in a mixer to generate a pooled drug substance which can be further diluted into different formulation to generate the drug substance (DS).

Step 3: Drug Product. Sterile filtration with filling and sealing in a blow fill seal (BFS) equipment. This step starts with sterile filtration, filling and sealing of DS, into vials. DS is filled into vials at a target fill weight of 2.0 mL per vial. All sealed vials are visually inspected for defects and sampled for container closure integrity by leak testing before labeling. Representative vials are sampled and tested for release.

### Example 13: Method of Treating a Wound

The fetal support tissue product of Example 1 is applied to a patch. The patch is applied directly to the wound for a period of time sufficient to treat the wound.

### Example 14: Method of Treating a Herniated Disc

The fetal support tissue product of Example 1 is formulated as an injection. The formulation is injected at the site of the herniated disc. Treatment is continued until a therapeutic effect is observed

### Example 15: Method of Treating Osteoarthritis

The fetal support tissue product of Example 1 is formulated as an injection. The formulation is injected into an arthritic joint. Treatment is continued until a therapeutic effect is observed.

1. A method of preparing a fetal support tissue product, comprising:
(a) cryopulverizing the fetal support tissue to generate a cryopulverized fetal support tissue;
(b) extracting the cryopulverized fetal support tissue in an excipient to generate an extract; and
(c) sterilizing by filtration the extract using a membrane having a pore size of about 0.6 μm or less followed by using a membrane having a pore size of about 0.4 μm or less;
wherein the fetal support tissue product is produced.

2. The method of claim 1, wherein the sterilizing by filtration is using a membrane having a pore size of about 0.45 μm followed by using a membrane having a pore size of about 0.2 μm or less.

3. The method of claim 1, wherein the cryopulverizing comprises pulverizing the fetal support tissue in liquid nitrogen.

4. The method of any one of claims 1-3, wherein the cryopulverizing comprises pulverizing the fetal support tissue to a fine powder.

5. The method of any one of claims 1-4, wherein the fetal support tissue comprises placenta, umbilical cord, placental amniotic membrane umbilical cord amniotic membrane, chorion, or amnion-chorion, or any combinations thereof.

6. The method of any one of claims 1-5, wherein the fetal support tissue comprises umbilical cord and placental amniotic membrane.

7. The method of any one of claims 1-6, wherein the excipient is saline, water for injection (WFI), or any combination thereof.

8. The method of any one of claims 1-7, wherein the excipient is WFI.

9. The method of any one of claims 1-7, wherein the excipient is saline.

10. The method of any one of claims 1-9, further comprising following step c) centrifuging the fetal support tissue.

11. The method of claim 10, wherein the centrifuge speed is about 14,000 relative centrifugal force (rcf) or higher.

12. The method of any one of claims 10-11, further comprising diluting the fetal support tissue with an excipient following centrifugation.

13. The method of claim 12, wherein the excipient is WFI or saline.

14. The method of claim 13, wherein the excipient is WFI.

15. The method of claim 13, wherein the excipient is saline.

16. The method of any one of claims 12-14, wherein the fetal support tissue is diluted by a factor of at least about 1.5-, 2.0-, or 2.5-fold.

17. The method of any one of claims 12-16, wherein the fetal support tissue is diluted by a factor of between about 1.5- to about 3-fold.

18. The method of any one of claims 12-17, wherein the fetal support tissue is diluted by a factor of about 2-fold.

19. The method of any one of claims 12-18, wherein the diluted fetal support tissue comprises from about 1 μg/ml to about 150 μg/ml of Hyaluronan (HA).

20. The method of any one of claims 1-19, wherein the fetal support tissue product is anti-inflammatory, anti-scarring, anti-angiogenic, anti-adhesion, or promotes wound healing.

21. A pharmaceutical composition, comprising (a) the fetal support tissue product made by the method of any of claims of any of claims 1-20, and (b) a pharmaceutically-acceptable carrier.

22. The pharmaceutical composition of claim 21, wherein the pharmaceutically-acceptable carrier is selected from: carbomer, cellulose, collagen, glycerin, hexylene glycol, hyaluronic acid, hydroxypropyl cellulose, phosphoric acid, polysorbate 80, propylene glycol, propylene glycol stearate, saline, sodium hydroxide, sodium phosphate, sorbital, water, xanthan gum, or any combination thereof.

23. The pharmaceutical composition of claim 21 or 22, wherein the fetal support tissue powder product is administered or provided as a cream, lotion, ointment, ophthalmic solution, spray, paste, gel, film, or paint.

24. The pharmaceutical composition of any one of claims 21-23, wherein the pharmaceutical composition is anti-inflammatory, anti-scarring, anti-angiogenic, anti-adhesion, or promotes wound healing.

25. A method of treating a wound in an individual in need thereof, comprising administering the pharmaceutical composition of any one of claims 21-24 to the wound for a period of time sufficient to treat the wound.

26. The method of claim 25, wherein the wound is a corneal epithelial wound.

27. The method of claim 26, wherein the corneal epithelial wound was caused by a photoablation treatment.

28. The method of any of claims 24-25, wherein the wound is a dermatological condition selected from a dermal burn or a scar.

29. A method of treating a spinal condition in an individual in need thereof, comprising administering the pharmaceutical composition of any one of claims 21-24 to the individual for a period of time sufficient to treat the spinal condition.

30. The method of claim 29, wherein the spinal condition is selected from a herniated disc, spinal adhesion, facet joint osteoarthritis, radiculopathy, a spinal cord injury, or discitis.

31. A method of treating an arthritic condition in an individual in need thereof, comprising administering the pharmaceutical composition of any one of claims 21-24 to the individual for a period of time sufficient to treat the arthritic condition.

32. The method of claim 31, wherein the arthritic condition is selected from osteoarthritis, rheumatoid arthritis, septic arthritis, ankylosing spondylitis, or spondylosis.

33. A method of regenerating or repairing bone, tissue or cartilage in an individual in need thereof, comprising administering or providing the pharmaceutical composition of any one of claims 21-24 to the individual for a period of time sufficient to regenerate or repair bone, tissue or cartilage.

34. The method of claim 33, wherein the pharmaceutical composition is administered or provided as a patch.

35. The method of claim 33, wherein the pharmaceutical composition is administered or provided as a wound dressing.

36. The method of any one of claims 1-35, comprising pooling the fetal support tissue product with at least one additional fetal support tissue product.

37. The method of claim 36, wherein the fetal support tissue product and the at least one additional fetal support tissue product comprise fetal support tissues derived from at least two different subjects.

38. The method of claim 36, wherein the fetal support tissue product and the at least one additional fetal support tissue product comprise fetal support tissues derived from at least five different subjects.

39. The method of claim 36, wherein the fetal support tissue product and the at least one additional fetal support tissue product comprise fetal support tissues derived from at least fifteen different subjects.

40. The method of claim 36, wherein the fetal support tissue product and the at least one additional fetal support tissue product comprise fetal support tissues derived from at least forty-five different subjects.

41. The method of any one of claims 1-40, comprising filling the fetal support tissue product into a container.

42. The method of claim 41, comprising sealing the container.

43. The method of claim 41 or 42, wherein the filling and sealing are carried out aseptically.

44. The method of any one of claims 41-43, wherein the filling and sealing are carried out aseptically and in a single continuous process without human intervention.
